(19) United States
(12) Patent Application Publication
(10) Pub. No.: US 2006/0068412 A1
(43) Pub. Date: Mar. 30, 2006

(54) Integrated multistep bioprocessor and sensor

(76) Inventor: Cha-Mei Tang, Potomac, MD (US)

Correspondence Address: MCDERMOTT, WILL & EMERY, 4370 LA JOLLA VILLAGE DRIVE, SUITE 700, SAN DIEGO, CA, 92122, US

(21) Appl. No.: 11/074,329

(22) Filed: Mar. 4, 2005

Related U.S. Application Data
(60) Provisional application No. 60/550,568, filed on Mar. 5, 2004.

(51) Int. Cl. C12Q 1/68; C12M 1/34

(52) U.S. Cl. 435/6; 435/287.2

(57) ABSTRACT

The invention provides an integrated biosensor. The integrated bioprocessor consists of an integrated capture chamber having an analyte recognition coating and a structure supporting analyte detection, analyte growth and target nucleic acid detection. The integrated capture chamber can consist of a waveguide, a capillary tube, a mixing flow chamber or an integrated combination thereof. The integrated capture chamber also can contain an antibody or other recognition species as an analyte recognition coating, an illumination source, a radiation detector, a microfluidics handling system, a second chamber for target nucleic acid detection or a combination thereof. Also provided is an integrated biosensor. The integrated biosensor consists of an integrated capture chamber having an analyte recognition coating, an illumination source, a radiation detector and a structure supporting analyte detection, analyte growth and target nucleic acid detection. The integrated capture chamber can consist of a waveguide, a capillary tube, a mixing flow chamber or an integrated combination thereof. The integrated capture chamber also can contain an antibody as an analyte recognition coating, a microfluidics handling system, a second chamber for target nucleic acid detection or a combination thereof.

This application is based on, and claims the benefit of, U.S. Provisional Application No. 60/550,568, filed Mar. 5, 2004, entitled “Integrated Multistep Bioprocessor and Sensor,” the entire disclosure of which is incorporated herein by reference.

### BACKGROUND OF THE INVENTION

This invention relates generally to methods and devices for processing and detecting biological particles and, more specifically to an integrated biosensor and processing methods that allow the efficient and sensitive detection of biological particles and components such as bacteria, spores, oocysts, cells, viruses, and parts thereof.

Even with improved methods for detecting pathogens in foods and environmental samples, microbiologists so mandated often face a “needle-in-a-haystack” challenge. In has been very difficult to detect small numbers of pathogens amid large numbers of harmless background microflora in a large and complex sample matrix. Traditional pathogen detection methods rely on culture enrichment, selective and differential plating, and additional biochemical and serological methods, making for analyses that may easily extend several days.

Recent events of anthrax bioterrorism have prompted the need to develop better methods to detect anthrax spores in environmental tests. Environmental sampling to determine the presence of Bacillus anthracis spores in letters and buildings is an important tool for assessing risk for exposure. During the extensive epidemiologic investigation of 2001-2002, >125,000 clinical and environmental specimens were collected and analyzed for B. anthracis. A majority of the specimens were environmental samples.

Currently, the Center for Disease Control and Prevention (CDC) recommends a two-step process for testing. The first test, a screening test, may be positive within 2 hours if the sample is large and contains a lot of B. anthracis spores, the organism that causes the disease anthrax. However, a positive reading on this first test must be confirmed with a second, more accurate test. This confirmation test, conducted by a more sophisticated laboratory, takes much longer. The length of time needed depends in part on how fast the bacteria grow, but results are usually available 1 to 3 days after the sample is received in the laboratory. Culturing protocol of environmental samples results in a very large number of non-anthracis colonies on the plates, so this protocol, too, has its drawbacks.

Some immunoassay technologies can be sensitive and fast, but they have not proven to be very specific for detection of anthrax. Most antibodies to anthrax spores are cross reactive to other Bacillus, such as B. thuringiensis, B cereus, and B. subtilus, present in the environment. Polymerase chain reaction (PCR) has been shown to be very specific in identifying B. anthracis and also has the ability to identify the species and strain under appropriate conditions. However, inhibitors can cause PCR to produce false negative results, particularly with environmental samples. In addition, PCR can also has a copy number detection limit below which the result is questionable. Further, the very small volume of fluid that can be processed by most PCR machines requires that an initial sample be split into a smaller portion for processing. This results in a loss of analyte and corresponding reduction in overall sensitivity, and is another cause of false negative results.

PCR was widely used to test suspect isolates as well as to screen environmental samples for the presence of B. anthracis during the 2001 anthrax attacks. Briefly, CDC reported that one hundred forty environmental specimens were analyzed by both culture and real-time PCR. A wide variety of samples were tested, including dust, paper towels, a syringe, vent filters, HVAC filters, vacuum cleaner debris, a cellulose sponge, and clothing; however, most samples were surface swabs (n=82). Of the 140 environmental specimens tested by both real-time PCR and culture, 35 were positive by both methods, 7 were positive by culture only, and 4 were positive by real-time PCR only (Letter from CDC, Evaluation and validation of a real-time polymerase chain reaction assay for rapid identification of Bacillus anthracis, Emerg. Infect. Dis. 8, 10 (2002)). Similar disagreement between real-time PCR and culture were described in CDC, Evaluation of Bacillus anthracis contamination inside the Brentwood Mail Processing and Distribution Center B District of Columbia, October 2001, MMWR Morb. Mortal. Wkly. Rep. 50, 1129-1133 (2001).

Similarly, the United States Department of Agriculture (USDA) reported on the processing of about 3,000 swab samples, 300 air samples, and 2,092 pieces of mail and other objects. None of the real-time PCR assays performed on extracted DNA were positive (a total of 4,639 reactions as of Sep. 15, 2002). The swab washings were full of dust and dirt. Even after laborious and reagent-consuming sample preparation, there were still so many inhibitor(s) present in the extracted DNA that they could only use 2-5% of the extracted DNA in the PCR reaction. Although the PCR machines are capable of detecting 5-10 spores, research at USDA showed that PCR inhibitors in environmental samples increased the limit of detection to 5000 spores (Higgins, J. A., Cooper, M., Schroeder-Tucker, L., Black, S., Miller, D., Kams, J., Manthey, E., Breeze, R. & Perdue, M. L. 2003. A field investigation of Bacillus anthracis contamination of USDA and other Washington, D.C. buildings during the anthrax attack of October 2001. Appl. Environ. Microbiol. 69, 593-599 (2002)).

The discrepancy between the ideal capabilities of PCR and environmental testing using PCR could be attributed to several factors such as the concentration of spores on contaminated surfaces, sample collection and preparation procedures, sample splitting, and the methods used for removing the sample from collection material. Furthermore, PCR- or immune-based tests do not distinguish viable from nonviable spores and can produce positive scores for samples that culture methods would define as negative. As a result, these methods are less useful for evaluating the success of disinfection techniques that do not remove nonviable spores.

Environmental testing for bioterrorism agents requires speed, sensitivity and specificity. Currently no single detection technology has all the desirable features. This disclosure proposes to integrate the best features of three different technologies: immunoassay, cell culture and real-time polymerase chain reaction (PCR), into one single test.

Most rapid immunoassays and DNA hybridization methods detect at best 500 CFU/g of target pathogens in ground beef (Demarco, D. R. & Lim, D. V. Detection of Escherichia coli O157:H7 in 10 and 25 gram ground beef samples with an evanescent wave biosensor with silica and polystyrene waveguides. J Food Protect. 65, 596-602 (2002); DeMarco, D. R. & Lim, D. V. Direct detection of Escherichia coli O157:H7 in unpasteurized apple juice with an evanescent wave biosensor. J. Rapid Methods and Automation in Microbiology. 9, 241-257 (2001); DeMarco, D. R., Saaski, E. W., McCrae, D. A. & Lim, D. V. Rapid detection of Escherichia coli O157:H7 in ground beef using a fiber-optic biosensor. J. Food Prot. 62, 711-716 (1999)), and 25 CFU per 100 ml of raw water after concentrating the raw water 100 fold (Shelton, D. & Karns, Quantitative detection of Escherichia coli O157 in surface waters by using immunomagnetic electrochemiluminescence. J. Appl. and Environ. Microbiol. 67, 2908-2915 (2001)).

Enzyme-based nucleic acid amplification methods, including the thermal cycling polymerase chain reaction (PCR), real-time PCR, isothermal nucleic acid amplification, nucleic acid sequence-based amplification (NASBA) and RNA, represent significant advances that have the potential to speed the overall analysis by replacing culture enrichment procedures with those that amplify specific nucleic acid sequences. These DNA and RNA based methods are highly specific. However, the detection limits fail to show improvement better than 10²-10³ CFU/g of food.

The reasons for such high limits of detection appear to be: (i) low levels of contaminating pathogens; (ii) high volumes (≧25 ml of sample) or high mass compared to amplification volumes (<10 μl); (iii) residual matrix components that inhibit enzymatic reactions and nonspecific amplification. Additional challenges include the need to confirm findings when nucleic acid sequences are detected from nonviable biological particles.

Separating, concentrating, and purifying food-borne microorganisms from sample matrices before undertaking nucleic acid amplification steps improve the overall analysis. Such procedures are necessary when detecting viral agents from foods because, unlike those bacterial pathogens that can be cultured, viruses are inert in food matrices. Unfortunately, separating and concentrating bacterial pathogens from foods can prove difficult because, unlike many viruses, bacterial cells are highly sensitive to agents such as organic solvents and detergents that are used to remove matrix-associated interfering compounds.

Approaches for concentrating target biological particles should address three issues that plague environmental and food microbiologists. Namely, (i) how to separate pathogens from sample particulates; (ii) how to remove inhibitory compounds associated with the matrix, and (iii) how to reduce the sample size and also recover nearly 100% of the target organism(s).

In general, the goal is to take a 25-50 ml of sample, and concentrate the target biological particles into a volume about 0.1 ml, with high recovery of viable target microorganisms and full removal of matrix-associated inhibitory compounds. Centrifugation is a commonly used physical method to separate and concentrate target biological particles from complex sample matrices. Filtration is another important tool for concentrating target biological particles.

Immunomagnetic separation (IMS) is one biologically based concentration technique. IMS combines the use of monoclonal or polyclonal antibodies with magnetic spheres to select target cells from a mixed population. After allowing the antibody to bind target biological particles within a matrix, target biological particles are separated from mixtures by exposing them to a magnetic field. IMS has proved an effective tool for isolating several food borne pathogens, including Listeria monocytogenes, Escherichia coli O157:H7, and Salmonella species. However, even when IMS precedes nucleic acid amplification steps, detection limits are rarely better than 10³-10⁵ CFU/ml of the target bacteria in a food homogenate.

When considered together, many of the biological particles concentration methods are complex, expensive, and can be applied only to relatively low-volume samples. Although achieving a 50- to 100-fold sample concentration with recovery of 100% of the target biological particles and complete removal of all matrix-related inhibitory compounds is desirable, this goal is difficult to achieve with current technologies.

Even with the best concentration and purification schemes, residual matrix-associated inhibitors typically remain in final extracts. These inhibitors either prevent amplification, resulting in false-negative results, or else reduce its efficiency, resulting in poor detection limits. These inhibitory effects sometimes are more pronounced when target template levels are particularly low, which is precisely when one needs higher amplification efficiencies.

Nucleic acid amplification assays fail to differentiate live from dead cells. Culture enrichments prior to PCR do not fully overcome this problem because nucleic acids from dead pathogens may be detected even after such enrichments. Additionally, some immunoassays are limited to only a few micro-liters of the whole sample. The result is sample splitting, which reduces the number of analyte in the sample such that the analyte in test volume falls below the detection limit. The ideal situation is to be able to process the whole sample.

Thus, there exists a need for a device and methods that rapidly and efficiently process large biological samples and yield quantitative determinations of biological analytes. The present invention satisfies this need and provides related advantages as well.

### SUMMARY OF THE INVENTION

The invention provides an integrated biosensor. The integrated bioprocessor consists of an integrated capture chamber having an analyte capture surface and a structure supporting analyte detection, target nucleic acid detection and/or analyte growth. The integrated capture chamber can consist of a waveguide, a capillary tube, a mixing flow chamber or a combination thereof. The integrated capture chamber also can contain an antibody as an analyte recognition coating, an illumination source, a radiation detector, a microfluidics handling system, a second chamber for target nucleic acid detection or a combination thereof. Also provided is an integrated biosensor. The integrated biosensor can also provide analyte growth. The integrated biosensor consists of an integrated capture chamber having an analyte capture surface, an illumination source, a radiation detector and a structure supporting analyte detection, target nucleic acid detection and/or analyte growth. The integrated capture chamber can consist of a waveguide, a capillary tube, a mixing flow chamber or a combination thereof. The integrated capture chamber also can contain an antibody as an analyte recognition coating, a microfluidics handling system, a second chamber for target nucleic acid detection or a combination thereof.

### BRIEF DESCRIPTION OF THE DRAWINGS

FIG. 1 shows the multi-step biosensor that can perform a rapid whole organism(s) detection providing serotype information, followed by culturing in the cartridge providing viability information, and subsequently performing nucleic acid detection(s) providing genotype(s) or polymorphism(s) information according to one embodiment of the invention.

FIG. 2 shows a multi-step biosensor that can perform a rapid whole organism(s) detection providing serotype information, followed by performing nucleic acid detection(s) providing genotype(s) or polymorphism(s) information according to another embodiment of the invention.

FIG. 3 shows a multi-step biosensor that can culture captured or concentrated analyte(s) in the cartridge providing viability information, followed by performing nucleic acid detection(s) providing genotype(s) or polymorphism(s) information according to another embodiment of the invention.

FIG. 4 shows an example of the multi-step biosensor that can perform a rapid whole organism(s) detection using waveguide(s) providing serotype information, determination of viability as well as real-time PCR providing genotype information according to one embodiment of the invention.

FIG. 5 shows an example of the multi-step biosensor that can perform a rapid whole organism(s) detection using waveguide(s) providing serotype information, followed by real-time PCR providing genotype information according to another embodiment of the invention.

FIG. 6 shows an example of the multi-step biosensor that cultures the captured analyte(s) on the waveguides(s) to provide viability, followed by real-time PCR providing genotype information according to another embodiment of the invention.

FIG. 7 shows a capillary-based waveguide apparatus employed as an integrated biosensor and bioprocessor.

FIG. 8 shows a schematic illustrating the MHD Lorentz force F generated by the coupling of a magnetic field B and an electrical current I. Note that the configuration is that of a tube, or a channel, generating an in situ micropump that can be implemented by microfabcrication and micro-fluidics. h is the height and w is the width of the channel.

FIG. 9 shows a schematic illustrating MHD micro-fluidic switch. As P1 is switched on, P2 is also switched on to generate an equilibrium pressure to prevent flow from going from Arm 1 to Arm 2. As a result, flow into Arm 3 can be switched from Arm 1 to Arm 2 by switching the MHD micropumps.

FIG. 10 shows a photograph image of the packaging of MHD micropump. Left: packaged MHD circular micropump with electrical leads compared to the micro-fluidic chip and a US quarter dollar. Right: comparison with a US quarter demonstrates the compact size of the electromagnet and the chip.

FIG. 11 shows MHD micro-fluidic circuit implemented with glass-PDMS microfabrication.

FIG. 12 shows a thin film polyimide microvalve for flow control in microchannels. Illustration of microvalve open (no electrical field) and closed (applied voltage).

FIG. 13 shows a flow chart schematic for the detection of a target biological particles analyte.

FIG. 14 shows a side view of a capillary waveguide employed in an integrated biosensor and bioprocessor of the invention.

FIG. 15 shows a schematic of a sandwich immunoassay format for detection of a biological particle analyte.

FIG. 16 shows the relationship between Cy5 fluorescence signal and analyte cell numbers captured on a capillary waveguide in an integrated biosensor and bioprocessor of the invention.

FIG. 17 shows a plot of real-time PCR amplification of the lacZ gene. The Y axis indicates the fluorescence signal while the X axis indicates the amplification cycle. The number at the right shows the copies/μl in the lacZ standard. The capillary sets A to D correspond to those in FIG. 16.

FIG. 18 shows the genetic locations of target genes for biosensor assays on E. coli O157:H7 chromosome as described in Pema et al. Nature 409:529-533, (2001).

FIG. 19 shows a growth curve of E. coli O157 in a biosensor capillaries (1.66×7 mm) and regular test tubes (15×125 mm).

FIGS. 20a-c are schematic representations of a top view, side view and end view, respectively, of a mixing flow-through sensor according to one embodiment of the invention.

FIG. 21a-h are cross-sectional representations of the waveguide according to several embodiments of the invention.

FIGS. 22a and b are schematic representations of the top views of the compact mixing flow-through sensors according to other embodiments of the invention, where the side walls of the mixing flow chamber have different shapes.

FIG. 23a and b are cross-sectional representations of a mixing flow-through sensor according to one embodiment of the invention at two axial locations. The body of the mixing flow channel has a three-dimensional variation.

FIG. 23c and d are cross-sectional representations of a mixing flow-through sensor according to an embodiment of the invention at two axial locations. The body of the mixing flow channel has another three-dimensional variation.

FIG. 24a and b are cross-sectional representations of a mixing flow-through sensor according to one embodiment of the invention at two axial locations. The radiation transmissive top surface of the mixing flow chamber is also the waveguide and the mixing is achieved by three-dimensional undulating bottom and side surfaces of the mixing flow chamber.

FIG. 25a and b are schematic representations of a compact mixing flow-through sensor according to one embodiment of the invention at two different axial locations. The mixing flow chamber contains two waveguides and they are illuminated by two radiation sources. The mixing flow is produced by the undulating side walls in combination with the waveguides.

FIG. 26 present side view of a mixing flow-through sensor according to another embodiment of the invention. The sensing system 900 comprises waveguide members 901 that are situated inside elongated body of mixing flow chamber 940. The end of the waveguide 903 is unobscured by the waveguide wall 934 to let the emission light out to the detector. Elongated body 940 includes top transmissive member 920 and bottom light absorbing member 930 and an inlet 960 and outlet 961. The bottom wall member 930 is undulating. The excitation light 950 is collimated but not perpendicular to the long direction of the waveguide. The mixing flow is produced by the undulation of the bottom wall only in combination with the waveguide.

FIG. 27a-c are schematic representations of a top view, side view and end view of a multi-analyte mixing flow-through sensor according to one embodiment of the invention, respectively. There is one waveguide in each mixing flow chamber.

FIG. 28a-c are schematic representations of a top view, side view and end view of a flow-through sensor according to one embodiment of the invention, respectively. There are many waveguides in the mixing flow chamber. The flow is perpendicular to the length of t he waveguide. The mixing of the flow is produced primarily by the waveguide. This embodiment can be for detection of a single analyte or multi-analyte. Two detector systems can be used.

FIG. 29 is a side view of the multi-waveguide flow-through sensor where the waveguide are positioned to further enhance mixing of the fluid as it flows pass the waveguides.

FIG. 30a-c are schematic representations of a top view, side view and end view of a multi-analyte flow-through sensor according to one embodiment of the invention, respectively. There are a number of mixing flow chambers. There are many waveguides in each mixing flow chamber. The flow is perpendicular to the length of the waveguide. Again, the mixing of the flow is produced primarily by the waveguide.

FIG. 31 shows the options of the fluid flow for the embodiment shown in FIGS. 11a, 11b and 11c.

FIG. 32a-c are schematic representations of the top view, side view and end view of a curved mixing flow chamber and curved waveguide.

FIG. 33 is a top representation of a multiple mixing flow-through sensor according to one embodiment of the invention with curved surfaces.

FIG. 34 is an end view of a schematic representation of a multi-analyte mixing flow-through sensor according to one embodiment of the invention.

FIG. 35a and b are schematic representations of a side view and end view of a mixing flow-through sensor according to one embodiment of the invention where the mixing is accomplished by moving parts.

FIG. 36a and b are schematic representations of the side view and end view of a mixing flow-through sensor according to one embodiment of the invention where the mixing is accomplished by moving parts.

FIG. 37 is a schematic representation of a end view of a mixing flow-through sensor according to one embodiment of the invention where the mixing is accomplished by applying an electric field in part of the flow channel.

FIG. 38a-c are the bottom, top and end views of the mixing flow-through sensor according to one embodiment of the invention where the fluid is guided to flow in a spiral pattern around the waveguide and the fluid is mixed at the sides of the waveguide.

FIG. 39a-d are the bottom, top, end view at one axial location and end view at another axial location of the mixing flow-through sensor according to one embodiment of the invention where the fluid is guided to flow to the left and right of the waveguide and the fluid is mixed at the sides of the waveguide.

### DETAILED DESCRIPTION OF THE INVENTION

This invention is directed to an integrated biosensor and bioprocessor. The integrated biosensor can process large sample volumes or amounts. One embodiment of the integrated biosensor is the ability to capture target organisms as an analyte for measurement. Whole organisms or components thereof can be captured, processed and detected in a multistep integrated fashion. Detection can be either quantitative or qualitative determinations for the number, type or other measurable attribute of the organism. Genetic identification of a target organism analyte is one example of a measurable attribute that can be determined using the integrated biosensor of the invention. The integrated biosensor can employ methods that reduce nucleic acid amplification inhibitors. Additionally, the integrated sensor of the invention can be employed to verify viability and can be used with large sample sizes.

In one specific embodiment, the above described functions as well as and other capabilities of the integrated biosensor of the invention can be accomplished by, for example, using an integrated biosensor and bioprocess having a cartridge to capture and manipulate the analyte and an instrument to detect the content of the cartridge. The cartridge can consist of an analyte capture member and a mixing flow chamber. The cartridge also can include a nucleic acid test chamber. Additionally, the integrated biosensor also can include at least one optical detector element or at least one illumination element or both. The integrated biosensor can include, for example, data acquisition and electronics element. Such elements can additionally include, for example, software data analysis and display element.

The invention also provides a method that can be used, for example, in conjunction with the integrated biosensor of the invention. The method consists of capturing a biological particle analyte, providing analyte capture information, culturing analyte in the chamber used for capture and to lysing the analyte in the chamber. Following lysis, nucleic acids endogenous to the analyte can be analyzed for identifying or other characteristics. The method can also include capturing a biological particle analyte, providing analyte capture information and lysis of a biological particle analyte in the chamber used for capture. Following lysis, nucleic acids endogenous to the analyte can be analyzed for identifying or other characteristics. Another method of the invention allows performance of nucleic acid analysis directly in the chamber used for capture or in an integrated test chamber.

As used herein, the term “analyte” is intended to mean a biological particle. Biological particles include, for example, cells, tissues, or organisms as well as fragments or components thereof. Specific examples of biological particles include bacteria, spores, oocysts, cells, viruses, bacteriophage, membranes, nuclei, golgi, ribosomes, polypeptides, nucleic acid and other macromolecules. Analyte complex is intended to mean a biological particle or a group of biological particles connected to analyte recognition coating and/or other components, such as proteins, DNA, polymers, optical emission detection reagent, etc.

Analyte recognition coating or elements are useful for selectively attaching or capturing a target analyte to a waveguide. Attachment or capture includes both solid or solution phase binding of an analyte to an analyte recognition coating. An analyte is attached or captured through a solid phase configuration when the analyte recognition coating or element is immobilized to a waveguide when contacted with an analyte. An analyte is attached or captured through a solution phase configuration when the analyte recognition coating or element is in solution when contacted with an analyte. Subsequent immobilization of a bound analyte-analyte recognition coating or element complex to a waveguide completes attachment or capture to the waveguide. In either configuration, either direct or indirect immobilization of the analyte recognition coating or element to a waveguide can occur. Direct immobilization refers to attachment of the analyte recognition coating or element to a waveguide allowing for capture of an analyte from solution to a solid phase. Immobilization of the analyte recognition coating or element can be directly to a waveguide surface or through secondary binding partners such as linkers or affinity reagents such as an antibody. Indirect binding refers to immobilization of the analyte recognition coating or element to a waveguide Analyte recognition element can form an analyte capture complex and become attached to the analyte capture surface on the waveguide.

Moieties useful as an analyte recognition coating or element in the invention include biochemical, organic chemical or inorganic chemical molecular species and can be derived by natural, synthetic or recombinant methods. Such moieties include, for example, macromolecules such as polypeptides, nucleic acids, carbohydrate and lipid. Specific examples of polypeptides that can be used as an analyte recognition coating or element include, for example, an antibody, an antigen target for an antibody analyte, receptor, including a cell receptor, binding protein, a ligand or other affinity reagent to the target analyte. Specific examples of nucleic acids that can be used as an analyte recognition coating or element include, for example, DNA, cDNA, or RNA of any length that allow sufficient binding specificity. Accordingly, both polynucleotides an oligonucleotides can be employed as an analyte recognition coating or element of the invention. Other specific examples of an analyte recognition coating or element include, for example, gangilioside, aptamer, ribozyme, enzyme, or antibiotic or other chemical compound. Analyte recognition coatings or elements can also include, for example, biological particles such as a cell, cell fragment, virus, bacteriophage or tissue. Analyte recognition coatings or elements can additionally include, for example, chemical linkers or other chemical moieties that can be attached to a waveguide and which exhibit selective binding activity toward a target analyte. Attachment to a waveguide can be performed by, for example, covalent or non-covalent interactions and can be reversible or essentially irreversible. Those moieties useful as an analyte recognition coating or element can similarly be employed as an secondary binding partner so long as the secondary binding partner recognizes the analyte recognition coating or element rather than the target analyte. Specific examples an affinity binding reagent useful as a secondary binding partner is avidin, or streptavidin, or protein A where the analyte recognition coating or element is conjugated with biotin or is an antibody, respectively. Similarly, selective binding of an analyte recognition coatings or element to a target analyte also can be performed by, for example, covalent or non-covalent interactions. Specific examples of a biochemical analyte recognition coating or element is an antibody. A specific example of a chemical analyte recognition coating or element is a photoactivatable linker. Other analyte recognition coatings or elements that can be attached to a waveguide and which exhibit selective binding to a target analyte are known in the art and can be employed in the device, apparatus or methods of the invention given the teachings and guidance provided herein.

As used herein, the term “analyte capture surface” is intended to mean a structure that has a surface coated with an analyte recognition coating. An analyte can be captured on the analyte detection substrate. Structures useful as an analyte detection substrate in the invention include, for example, waveguides, flat gold surfaces, colloidal gold, colloidal silver, plastic micro beads, magnetic micro beads, nano-holes, nano-column arrays, micro-holes, micro-column arrays, cantilevers, etc. An analyte detection substrate can be of any shape, dimensions, and texture suitable for its function, including, for example, flat, round, angled, smooth or rough surfaced, hollow, patterned, or any other shape, dimension, or texture. An analyte detection substrate can be composed of any material or combination of materials suitable for its function, including, for example, glass, polymer, fibers, composite materials, or any other materials.

A sample containing an analyte can be, for example, a liquid, solid or gas medium. Liquid mediums include, for example, water, buffer, serum, whole blood, urine, sweat, sputum, saliva, milk and juices. Analytes in air may be placed into liquid medium by mixing air through the liquid. Analytes in solid samples such as food, soil, fat, and other solids can be dissolved or suspended into liquid by homogenization. Large particles and lipids can be eliminated from samples before analysis to increase efficiency.

As used herein, the term “concentration” is intended to mean a process that increases the amount of an analyte per unit volume of liquid. Therefore, the term includes methods to collect an analyte of interest into a small liquid volume usable in the multi-step biosensor.

As used herein, the term “target” when used in reference to an analyte or component thereof is intended to mean the organism, cell, macromolecule, biochemical compound or chemical compound that is sought to be identified. A target molecule therefore includes a biological particle as well as any measurable marker contained on or within the biological particle. Target molecules or markers can include, for example, nucleic acids, polypeptides, carbohydrate, lipid, other macromolecules or macromolecular complexes as well as organic compounds or inorganic compounds. A specific example of a target molecule of the invention is a nucleic acid, such as a genome, gene, mRNA or rRNA that is measured by a nucleic acid detection method following capture of the biological particle analyte.

As used herein, the term “transduction” is intended to mean the production of a measurable signal. Measurable signals include, for example, physical, chemical, electrical, optical, thermal, or magnetic signals and can be used to qualitatively or quantitatively indicate the presence, abundance or both the presence and abundance of an analyte.

As used herein, the term “detection” is intended to mean a measurement of a transduced signal. Detection of a signal therefore provides an indication, such as a numerical value or visible criteria, for example, of the presence or absence of a target analyte, or the quantity of a target analyte. When used in reference to a nucleic acid target molecule, the term refers to the measurement of a nucleic acid sequence, such as DNA or RNA. For example, nucleic acids can be specifically detected by sequence specific hybridization. Particular methods useful for specific detection of nucleic acids include, for example, hybridization and can be used together or apart from nucleic acid amplification methods such as polymerase chain reaction (PCR), a thermal cycling process, or by strand displacement amplification (SDA), an isothermal amplification, before detection. Reverse transcription DNA (RT-DNA) can also be amplified by PCR or SDA before detection.

As used herein, the term “waveguide” is intended to mean a structure that facilitates the transmission of electromagnetic radiation. Transmission can be facilitated by, for example, using materials that assist electromagnetic radiation propagation along or within a waveguide structure. Transmission also can be facilitated by, for example, imparting directionality on the transmission, reducing loss of an signal, minimizing scatter emission, focusing of a transmitted electromagnetic propagation beam or capture of electromagnetic signal. Other modes of facilitation for electromagnetic radiation transmission are well known to those skilled in the art and also are included within the meaning of the term as it is used herein. Therefore, a waveguide functions, for example, as a conduit of electromagnetic radiation including, for example, optical signals.

The electromagnetic radiation can be guided in the waveguide when the index of refraction of the waveguide is higher than its surrounding. To operate the waveguide surrounded by air, the index of refraction of the waveguide needs to be greater than 1.0. Index of refraction of water is 1.33. Index of refraction of waveguide greater than water would be preferable. For example, the index of refraction of glass and many polymers is about 1.5.

All forms of electromagnetic radiation from infrared to ultraviolet region can be used in connection with a waveguide of the invention. Such forms include, for example, electromagnetic wavelengths within the ultraviolet region of the spectrum at about 50-380 nm, the visible portion at about 380-780 nm, the near-infrared region at about 780-3000 nm, the intermediate infrared region at about 3000-8000 nm as well as longer and shorter wavelengths. Additionally, a waveguide can be composed of, for example, any material and consist of any structural form or shape so long as it facilitates the transmission of electromagnetic radiation. Exemplary materials that can be utilized in a waveguide include, for example, high index of reflection, low transmission loss and non-fluorescent materials such as glass or polystyrene. Other exemplary materials include, for example, polymethyl-methacrylate (PMMA) and quartz. A waveguide can be composed of a single material, mixtures of different materials, rations of the same material or two or more separated materials, for example. Given the teachings and guidance provided herein, those skilled in the art will know, or can determine, whether a waveguide made of a single material, a mixture or distinct and separable materials are beneficial for a particular application.

As used herein, the term “mixing flow chamber” is intended to mean an enclosed or compartmented space that allows the flow of fluids or particulate bodies or other substances that move like fluids and mixing of constituents contained within the chamber. Therefore, a mixing flow chamber allows fluids or other substances with a fluid-like movement behavior to move with a change of place among the constituent particles or parts. The change of place can be continuous or non-continuous, as well as regular or sporadic motions. Accordingly, the term “flow chamber” as it is used in reference to a mixing flow chamber refers to the compartmented space in which fluid can flow through. The portion of a flow chamber that compartmentalizes a flow space can be, for example, a body or wall structure or one or more surfaces forming an encapsulated space. A mixing flow chamber and the flow space corresponding to the flow chamber can take on a variety of sizes and shapes so long as fluid or other substances with fluid-like movement behavior can change place relative to a position in the mixing flow chamber or relative to other constituents of the fluid or fluid-like substances and so long as the chamber can be configured to produce mixing. Mixing can result by modification of fluid flow directionality or periodicity using, for example, a waveguide, a structure of the flow chamber, other structures that augment mixing of fluid, or any combination thereof. Specific examples of a structure of the flow cell include an undulating surface or surfaces of the flow chamber or a non-elongated flow cell shape such as an arc, circle, sphere, periodic shape or one or more combinations thereof. Specific examples of such other structures that augment mixing include a free or unattached waveguide, actuation of movable objects, application of, for example, an electric field or heat, or one or more combinations thereof. Other means well known in the art that facilitate mixing of fluid or fluid-like substances similarly can be used to configure a flow chamber given the teachings and guidance provided herein.

As used herein the term “radiation transmissive” when used in reference to a material, device or apparatus of the invention is intended to mean that the material of a medium that allows electromagnetic radiation to pass or be conveyed through that medium. The term includes a medium that allows passage or conveyance of all wavelengths of electromagnetic radiation including, for example, wavelengths within the ultraviolet region of the spectrum at about 50-380 nm, the visible portion at about 380-780 nm, the near-infrared region at about 780-3000 nm, the infrared region at about 3000-8000 nm as well as longer and shorter wavelengths. Therefore, a radiation transmissive surface functions to admit the passage of radiation.

As used herein, the term “portion” as it is used in reference to a waveguide is intended to mean a part of a waveguide. Therefore, the term refers to less than the whole or entire waveguide.

As used herein, the term “surface” is intended to mean the exterior or outside, or the interior or inside, of an object or body. Therefore, depending on the reference orientation, the term surface can refer to an outer boundary of a structure, an inner boundary of a structure or the entire thickness of a structure when, for example, the structure is a partition dividing contents between spatial locations. A surface also can refer to a portion of a structure. For example, a waveguide can exhibit multiple surfaces. A reference to a surface, as it is used herein, includes some or all of a face of a surface as well as the entire face of a surface. Therefore, the term is intended to include that part of something that is presented to a reference view, a reference orientation, or a reference component of the device or apparatus of the invention. Moreover, it is to be understood that for an optically transmissive structure, a surface can refer to either the exterior, interior or both surfaces when used in reference to optical properties. For example, a reflective surface can be physically contained on an external surface of, for example, a mixing flow chamber, but will also reflect optical signals internally because of the transmissive nature of the structure. Given the teachings and guidance provided herein, those skilled in the art will understand whether external or internal surfaces are functionally distinguishable or alike when reference is made to a particular coating, property or structure.

As used herein, the term “radiation power” is intended to mean the amount of energy associated with the reference radiated in one second. Therefore, the term radiation power when used in reference to a measurement as a function of radiation wavelength refers to the amount of radiation energy collected by the detector per second. Similarly, the term “instantaneous radiation power” is intended to mean the amount of energy associated with the ration in a short sampling time period. The term instantaneous radiation power is used in reference to the amount of radiation energy collected by the detector in a short sampling time period.

As used herein, the term “plurality” is intended to mean two or more referenced signals. Therefore, the term as it is used herein refers to a population of two or more different signals. A plurality can be small or large depending on the design of the apparatus or need of the user. Small pluralities can include, for example, sizes of 2, 3, 4, 5, 6, 7, 8, 9 or 10 or more different signals. Large populations can include, for example, a composite number greater than about 12 or more different signals including tens or hundreds of different signals. Similarly, when used in reference to molecular species or components of an device or apparatus of the invention, the term “plurality” as it is used herein refers to two or more molecules, species or units of the referenced entity.

As used herein, the term “emission detection reagent” is intended to mean a molecule or a material that can emit a specific or characteristic optical or electromagnetic signal, including, for example, selectively scatter, reflect, transmit or emitted electromagnetic radiation. Some emissive detection reagents known in the art can be luminescent, fluorescent or phosphorescent material. The term “luminescence” when used in reference to an emission detection reagent of the invention is intended to mean production of electromagnetic radiation by a chemical or biochemical that is used as or produced by a detection reagent. A luminescent detection reagent can include, for example, luciferase. The term “chemiluminescent” refers to the production of light when the excitation energy derives from a chemical reaction, in contrast to the absorption of photons, in fluorescence. Bioluminescent refers to a subset of chemiluminescence, where the light is produced by biochemical reaction, such as from fireflys, bacteria and other organisms. Specific examples of organisms exhibiting bioluminescence include, for example, Vibrio fischeri, dinoflagellates and sea-fishes. A specific example of bioluminescence is the production of light by a firefly where the substrate Luciferin combines with the enzyme Luciferase and reactants ATP (adenosine triphosphate) and oxygen.

The term “fluorescence” when used in reference to an emission detection reagent of the invention is intended to mean light emission following absorption of energy from an external source of light. Fluorescent emission can be from a chemical or biochemical used as or produced by a detection reagent. The wavelength that is emitted is longer than the wavelength that is absorbed. Specific examples of fluorescent materials include colored dyes such as Cy-3, Cy-5, Alexa Fluor, green fluorescent protein (GFP), silicon nanoparticles, quantum dots, and a diverse collection of other materials well known in the art.

The term “phosphorescence” as it is used herein in reference to an optical emission detection reagent, is intended to refer to similar phenomenon as fluorescence except that the excited product is relatively more stable. Accordingly, the time until energy is released is longer compared to fluorescence, resulting in a glow after the excitation light has been removed. Phosphorescent emission also can be from a chemical or biochemical used as or produced by a detection reagent. Luminescence, fluorescence and phosphorescence and detection methods employing these phenomenon are well known in the art and can be found described, for example, at the URL lifesci.ucsb.edu/˜biolum/myth.html.

Other electromagnetic emission detection reagents include colloidal gold, colloidal silver, other colloidal metal plasmon resonant particles, grating particles, photonic crystals and the like. These as well as others are well known in the art and can similarly be employed in the apparatus or methods of the invention given the teachings and guidance provided herein.

The invention provides an integrated biosensor. The integrated bioprocessor consists of an integrated capture chamber having an analyte recognition coating and a structure supporting analyte detection, analyte growth and target nucleic acid detection. The integrated capture chamber can consist of a waveguide, a capillary tube, a mixing flow chamber or an integrated combination thereof. The integrated capture chamber also can contain an antibody as an analyte recognition coating, an illumination source, a radiation detector, a microfluidics handling system, a second chamber for target nucleic acid detection or a combination thereof.

Also provided is an integrated biosensor. The integrated biosensor consists of an integrated capture chamber having an analyte recognition coating, an illumination source, a radiation detector and a structure supporting analyte detection, analyte growth and target nucleic acid detection. The integrated capture chamber can consist of a waveguide, a capillary tube, a mixing flow chamber or an integrated combination thereof. The integrated capture chamber also can contain an antibody as an analyte recognition coating, a microfluidics handling system, a second chamber for target nucleic acid detection or a combination thereof.

Either the integrated biosensor or the integrated bioprocessor can be employed for either detection or identification of biological particles or both. Accordingly, the invention provides an integrated biosensor and bioprocessor.

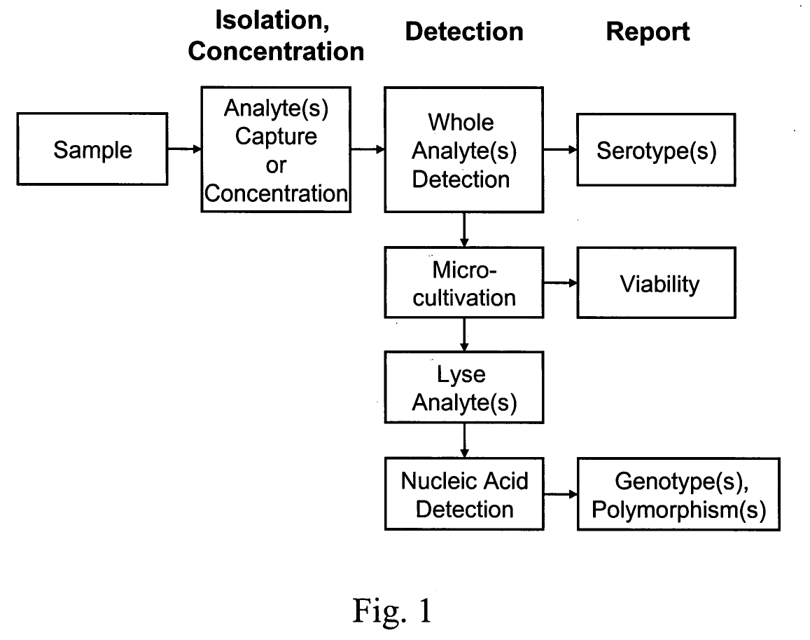

The invention provides a method and apparatus for identifying the serotype viability, genotype or polymorphism of an organism. The method and apparatus combine three separable processes into a single integrated process that can be performed in a single integrated apparatus. FIG. 1 describes the multi-step detection method. For example, a sample containing one or more analytes of interest can be captured on an analyte detection substrate or concentrated into a small liquid volume. A transducer method is applied to the captured analyte and the presence of the analyte produces a detectable signal. This first step of the integrated biosensor and bioprocessor provides a rapid and efficient result that can entail information regarding, for example, serotype or abundance of the analyte in the sample. The second step involves culturing the cells in the capture chamber. This second integrated step allows confirmation, for example, of the viability and increase the number of analytes within a sample. A further integrated step includes measuring a target molecule present on or within the analyte. This further step can be accomplished by, for example, lysing the analyte and detection of a nucleic acid specific to the target analyte. Detection of a target molecule provides, for example, confirmation of the analyte's genotype, including polymorphism variants thereof.

The procedures and the significances of the integrated biosensor and methods are exemplified below with reference to anthrax spore detection and detection of E. Coli in water. However, given the teachings and guidance provided herein, it will be understood by those skilled in the art that the integrated biosensor and methods of the invention can be employed with a wide range of biological particle analytes. Accordingly, the exemplary integrated apparatus and methods described below are equally applicable to other types of spores, bacteria, bacteriophage, cells, tissues and organisms.

An integrated biosensor of the invention can be constructed or employed to exhibit, for example, the following properties. Briefly, an integrated biosensor allows for rapid or sensitive detection of biological particles within a time of about 10 minutes to several hours. It can include a cell culture step that verifies the presence or viability of a biological particle analyte, including bacterial, viral, and spore-forming organisms. The cell culture step also can be employed to increase the number of organisms available for detection. Additionally, an integrated biosensor of the invention also can include a genetic test step for detection of the analyte. Such a genetic detection step is useful as a confirmation of a previous immunoassay result, and as such, further increases the specificity of the immunoassay, and provides specific DNA information. PCR or other nucleic amplification or detection method can be performed in an integrated fashion to yield a real-time result. These and other capabilities of an integrated biosensor of the invention allow, for example, quantitation of the number of analytes in the sample.

An integrated biosensor of the invention can be produced as a compact, self-contained, disposable test cartridge which safely confines the environmental sample and all test by-products. Additionally, an integrated biosensor of the invention can be automated in sample preparation or data acquisition or both. Accordingly, an integrated biosensor of the invention can be a portable apparatus for field use by first responders.

An integrated biosensor of the invention can achieve rapid speeds, efficiencies or accuracy useful in a variety of applications. Characteristics contributing to rapid, efficient or accurate results include, for example, inclusion in a test cartridge that is capable of performing at least the integrated functions of capture, growth and target molecule detection.

For example, a first integrated step is included that can employ a sensitive capture and detection assay, such as immunoassay. The capacity of the test cartridge can allow the processing of entire contents of a normal environmental sample, typically 2-30 ml, without sample splitting or the loss of sample analyte. This feature enables more accurate positive test results from a smaller number of biological particles. The capturing of analyte on a surface and flowing the rest of sample out of the detection chamber serves as a filter to remove environmental contaminants and inhibitors, and at the same time concentrates the analyte to a smaller volume for subsequent processing.

A second integrated step can employ culturing of the analyte or sample suspected of containing an analyte. Culturing produces more analyte material for subsequent analysis and also is useful for verifying viability of the analyte. In specific embodiments that include capture and detection of spore-forming organisms, such as anthrax, spores can first be germinated, followed by cell culture. Additionally, the integrated culture step amplifies viable organisms in the test cartridge. This will increase the concentration of target template for PCR and increase the sensitivity of the detection.

A third step of an integrated biosensor of the invention can include nucleic acid amplification and detection from the captured analytes. This step can provide specific gene based identification of an analyte. Other modes of target molecule detection also can be employed in this integrated step. For example, polypeptide targets can be detected by affinity binding assays using specific binding molecules. Specific binding molecules can include, for example, antibodies, receptors, ligands or antigens. Additionally, because of the integrated design of a biosensor of the invention, sample splitting for the nucleic acid amplification step will be reduced compared to other sample preparation methods.

Other characteristics and attributes of the integrated biosensor of the invention include, for example, a solution to a need for technology that combines efficient detection features, and as such, produces more sensitive and more specific results than available separable methods. These characteristics allow for both on-site rapid testing of analytes as well as for laboratory applications. The integrated biosensor addresses inherent problems resulting in both false negatives (for example, sensitivity, viability, inhibitors) and false positives (for example, cross-reaction, contamination) that affect other methods. Because of its efficiency and integrated nature, an integrated biosensor of the invention can more accurately and rapidly confirm the presence of pathological agents, including bioterrorism agents, without the cost and delay of sending sample to a biological safety level 3 (BSL3) laboratory for confirmation tests. Further, because the integrated biosensor can confirm analyte viability, it will enable real time monitoring of decontamination efforts. Additionally, by selectively capturing pathogenic spores from environmental samples and separating them from other biological particles in the sample, the integrated biosensor can provide a spore-derived vegetative cell lysate free of inhibitors for subsequent amplification reactions, such as PCR. Further, the integrated biosensor allows processing of whole sample wipes or swabs, which can lower the limit of detection. By capturing the spores in, for example, a capillary tube or flow-through mixing chamber, the lysate volume can be small and DNA concentration is high. Lysate sample dilution can therefore be reduced and the limit of detection can be further reduced.

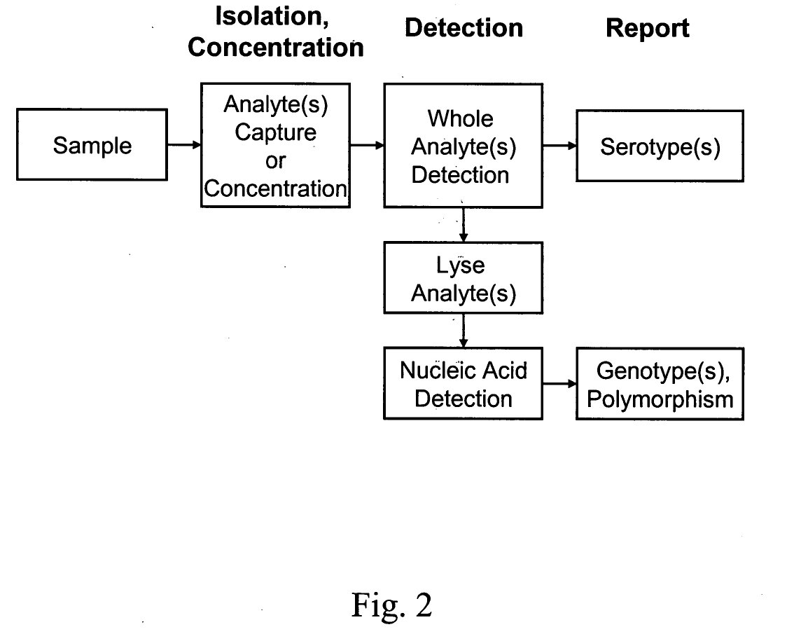

FIG. 2 exemplifies variation of the integrated biosensor and detection method of the invention. Briefly, a sample containing one or more analytes of interest is captured on an analyte detection substrate or concentrated into a small liquid volume. A transducer method is applied and the presence of the analyte produces a signal that is detected. The next step can be lysing the analyte or analytes, followed by detection of the target nucleic acid to provide confirmation of genotype or polymorphism.

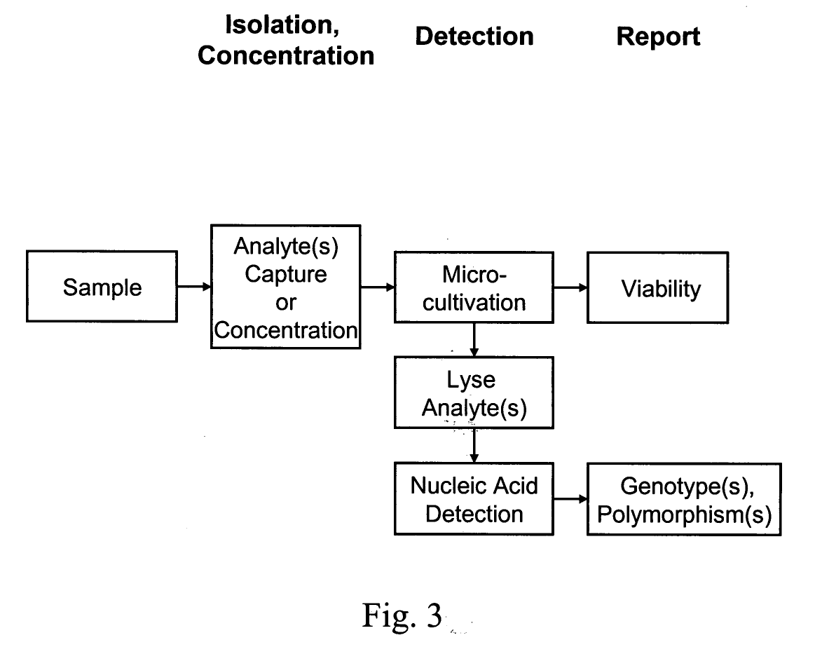

FIG. 3 exemplifies another variation of the integrated biosensor and detection method of the invention. A sample containing one or more analytes of interest is captured on an analyte detection substrate or concentrated into a small liquid volume. The analyte is cultured in the capture chamber. Culturing can be used to confirm viability and/or to increase the number of analytes present in the integrated biosensor. The next step can be lysing the analyte or analytes, followed by detection of the target nucleic acid to provide confirmation of genotype or polymorphism.

The description above exemplifies the use of antibodies as an affinity binding molecule for capture surface coating. However, other affinity binding molecules specific to an analyte of interest can be equally substituted for the antibody component described herein. Given the teachings and guidance described herein, those skilled in the art will know which other specific binding molecules are applicable for use in an integrated biosensor and methods of the invention.

A capture surface coating can be placed, for example, on a variety of different analyte detection substrates as described previously. The transduction method similarly can vary depending, for example, on the need of the user and the analyte detection substrate being employed. For example, a transduction method can employ optical intensity by the use of luminescent, fluorescent or phosphorescent materials. Transduction methods also can employ surface plasmon resonance (SPR) in conjunction with, for example, gold or silver surfaces having thicknesses appropriate for this well known procedure. Additionally, a transduction method also can employ a signal based on a change in physical shape of the surface. Specific examples of a transduction method employing changes in physical shape includes atomic force microscope (AFM) imaging or a physical change of a cantilever. Further, a transduction method applicable for use in the integrated biosensor and methods of the invention also can utilize flow of current based on connection of two electrodes based on contact formed by the captured analyte. Other transduction methods well known in the art also can be utilized in the integrated biosensor and methods of the invention.

The biosensor of the invention integrates the above described culture step inside the chamber that contains the captured analyte. This integration minimizes loss of analyte sample and maximizes availability of analyte material for subsequent analysis. The duration of the culture will vary depending on the need of the user, complexity of the sample and amount of the analyte in the sample. Briefly, the culture period can be sufficient to multiple an analyte population within a sample by a factor of about two- to ten-fold or greater. Analytes in lower abundance can be cultured longer periods whereas analytes in greater abundance can be cultured shorter periods. Additionally, for the specific embodiments where the analyte is a spore, the culture period should be sufficiently long to enable the germination of the spore, and preferably to enable germination and growth of the analyte for at least one to two generations. Given the teachings and guidance provided herein, those skilled in the art will known or can readily determine a culture duration sufficient for analyte detection in light of the sample and analyte to be detected.

Analyte lysing step can also be integrated in the biosensor apparatus of the invention. For example, cells lysis can be performed inside the chamber that contains the cultured cells. One method to lyse the cells includes heating the analyte to about 95° C. Another method includes addition of chemicals such as Triton X-100 detergent (Sigma), NP-40 detergent (Sigma), AL lysis buffer (Qiagen kit). Other methods well known in the art can similarly be employed for analyte lysis and are equally applicable in the integrated biosensor and methods of the invention.

Further, a nucleic acid detection step also can be integrated in the biosensor and methods of the invention. As described previously, target nucleic acid detection can provide results directed to identifying a genotype or polymorphism of the analyte. Single nucleotide polymorphism (SNPs) is one nucleic acid detection method well known in the art for detecting or identifying a variant of a cell or organism.

Target nucleic acid detection methods employed in the integrated biosensor and methods of the invention can use, for example, any transduction method and detection method to determine the genotype or SNPs of interest. For example, nucleic acid detection can include microtube based or on a nucleic acid detection substrate. Specific examples of such other nucleic acid detection substrates include waveguides, array chips, colored beads and the like. Similarly, nucleic acid detection can employ any of various methods well known in the art to produce a probe or specifically bind the probe to the target nucleic acid. For example, hybridization and amplification detection methods using labeled probes or primers are well known in the art and can be employed as one method of target nucleic acid detection in the integrated biosensor and methods of the invention. The nucleic acid detection can test for a single or multiple number of nucleic acid sequences for each analyte.

The integrated biosensor and methods of the invention can provide non-quantitative or quantitative results for the whole organism or for genetic sequences. The integrated biosensor and methods of the invention also can be used to measure one or more analytes. Measurements can occur in serial, parallel, simultaneously or in multiplex formats.

Other variations of the integrated biosensor and methods of the invention are exemplified below.

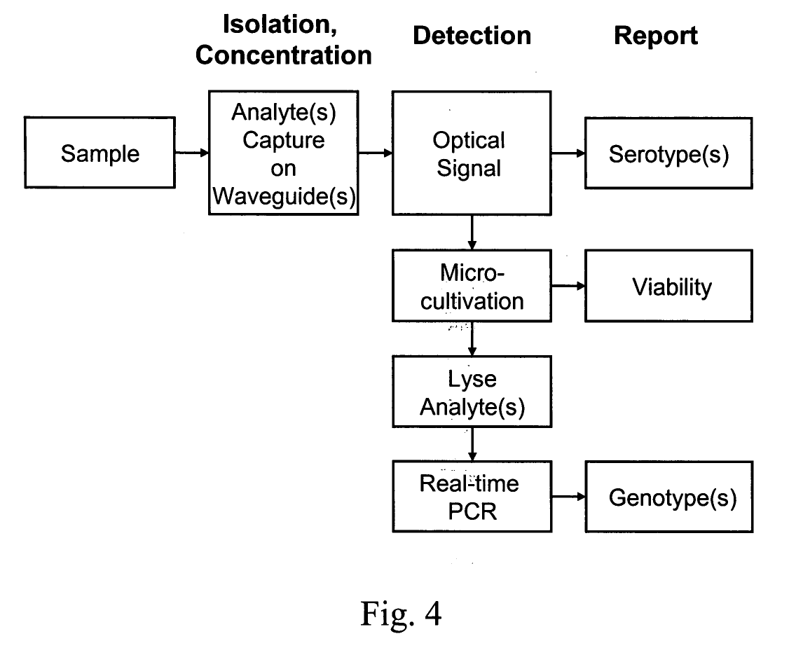

A further variation of an integrated biosensor and method of the invention is depicted in FIG. 4. In this variation, an analyte is captured on a waveguide. An optical signal is produced and detected to provide a rapid quantitative result of the serotype. The analyte is cultured and is followed by lysing in the analyte capture chamber. The nucleic acid sample is flown into the real-time PCR chamber. Flow-through mixing chambers, waveguides and optical signals are described further below.

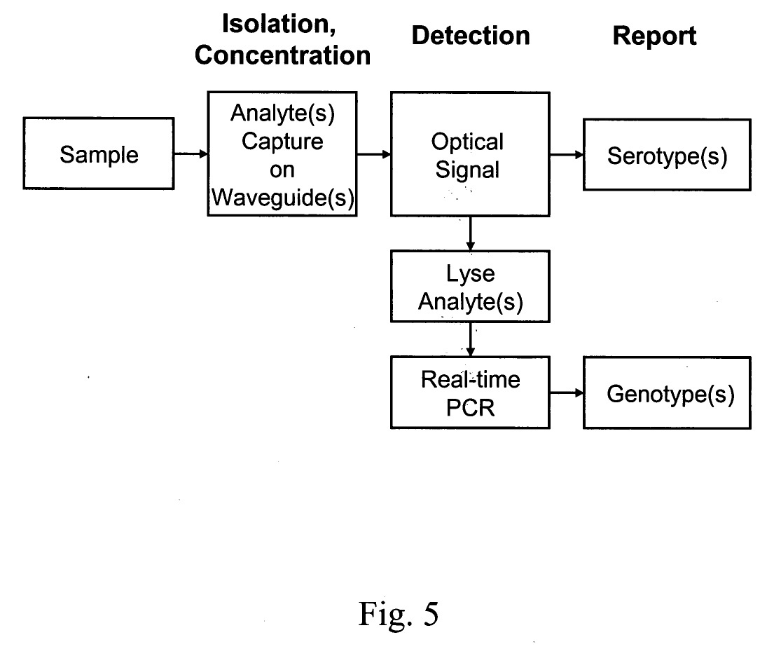

Still a further variation of an integrated biosensor and method of the invention is depicted in FIG. 5. The analyte is captured on a waveguide. An optical signal is produced and detected to provide a rapid quantitative result of the serotype. The analyte is lysing in the analyte capture chamber. The nucleic acid sample is passed into the real-time PCR chamber.

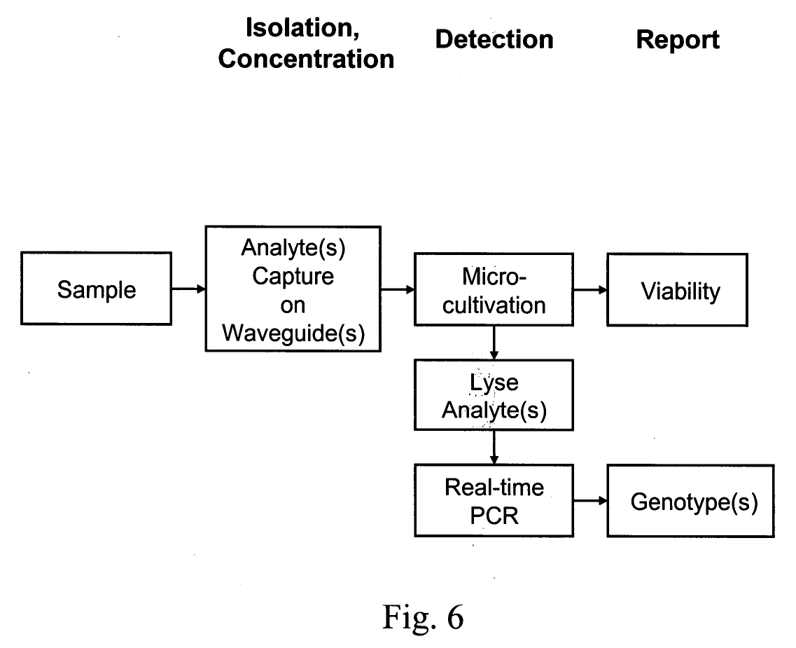

Another variation of an integrated biosensor and method of the invention is depicted in FIG. 6. The analyte is captured on the surface of waveguide. The analyte is cultured in capture chamber and is followed by lysing in the capture chamber. The product is passed into PCR chamber.

Given the teachings and guidance provided herein, those skilled in the art will understand that there are numerous different configurations of an integrated biosensor and method of the invention.

One device applicable for use as an integrated biosensor and bioprocessor of the invention includes an optical fiber biosensors based on evanescent wave excitation and detection such as that described in Anderson et al., IEEE Trans. on Biomed. Eng. 41, 578-584 (1994); Golden et al., On Biomed. Eng. 41, 585-591 (1994a), and Golden et al., Chemical, in 1796 SPIE Proceedings Series, pp. 2-8 (Meeting 8-9 Sep. 1992, in Boston, Mass.; published April 1993) (1994b). Another device that can be applicable for use as an integrated biosensor and bioprocessor of the invention includes a multi-analyte and multi-sample array biosensors using evanescent field excitation on planar waveguides such as that described in Feldstein et al., J. Biomed. Microdevices 1, 139-153 (1999); Rowe et al., Anal. Chem. 71, 433-439 (1999a); Rowe et al., Anal. Chem. 71, 3846-3852 (1999b), and Rowe-Taitt et al., Biosensors & Bioelectronics 14, 785-794 (2000).

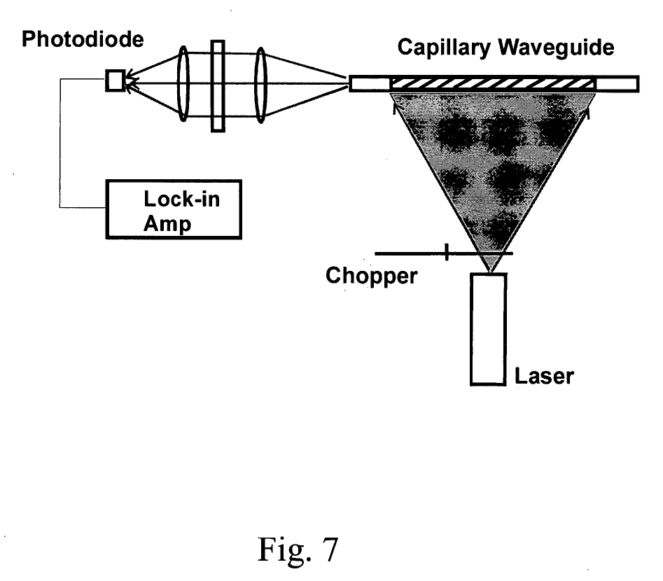

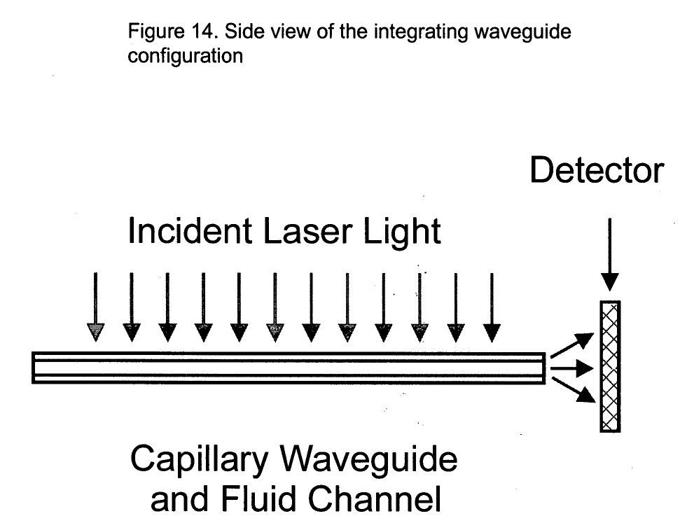

Another example of an integrated biosensor is shown in FIG. 7 and in FIG. 14, described below in the Example. Briefly, this embodiment on an integrated biosensor can utilize devices well known in the art and modified for capture, growth and detection, and can incorporate a growth or germination step integrated therein. A capillary tube or similar structures also can be employed as an integrated biosensor and bioprocessor of the invention. This tube and other similar structures or devices can be utilized to perform, for example, an immunoassay in a flow channel inside an optical waveguide, where the incident excitation light is perpendicular to the waveguide. The emitted fluorescence from, for example, a sandwich format immunoassay of the analyte is collected at one end of the waveguide. One configuration is that shown above in FIG. 7. Emitted light is coupled very efficiently into the waveguide and the signal is integrated by the geometry of the sensing component. The emitted light can be collected on a photo multiplier tube (PMT) or photodiode. As a result of this configuration, the signal from a relatively large surface is integrated and measured at a single-point. The waveguide can be as simple as a capillary tube, or can take on a more complex geometry. The analyte-sensing surface can be formed by coating the inside of the capillary with a biomolecular recognition species, an antibody for example. Micro-fluidic flow channels can be used to introduce the sample and the labeled recognition molecules over the waveguide. These sensors have demonstrated about two orders of magnitude greater sensitivity than NRL's previous technology based on evanescent wave fiber optics and planar arrays (Ligler et al., Anal Chem. 74, 713-719 (2002)).

Advantages of the above described capillary waveguide based integrated biosensor and bioprocessor include, for example, direct illumination of the waveguide produces better excitation of the fluorophores than evanescent waves. Also, a signal generated along the entire waveguide surface is coupled to the detector at one end of the waveguide. The amplitude of the signal can be increased by increasing the signal-generating surface area (i.e., using a larger capillary tube), and background noise from the excitation light can be minimized by optimal location of the components and by employing wavelength filters. The integrated biosensor of the invention shown in FIG. 7 demonstrates these advantages and consists of an excitation source, the capillary holder, and the photomultiplier tube detector.

The structure of the capillary waveguide component of an integrated biosensor of the invention can be, for example, a round capillary with 0.7 mm (inner diameter). The capillary can be, for example, coated with Teflon on the outside and used as an optical waveguide to transmit an optical signal, and at the same time, provide a flow channel for sample and analyte. An excitation laser beam can be oriented 90° relative to the optical signal emission path. A detector can be, for example, a Hamamatsu HC-120-05 photo-sensor (PMT). Further, the detector can receive emission from the end of the capillary after passing through a bi-convex collimating lens, band pass filter, a long pass filter, and a focusing lens. The PMT output can be connected to an input of a Lock-in Amplifier. External reference input can be provided from the optical chopper trigger output. Capture, binding and other analysis can be performed as exemplified in the Example below.

Augmentations or modifications to the above devices utilized in an integrated biosensor of the invention or to the above capillary based integrated biosensor can include, for example, changing only the capillary tube from a 0.7 mm inner diameter custom tube to a commercially available source with a 1.22 mm inner diameter while still using the same mounting or illumination and detection components. For example, an improved dose response can be obtained for the capture step with the above change in capillary diameter. Results show that a 10 pg/ml signal (average 19.5) using the larger diameter, which is significantly larger than the limit of determination at 8.2 (mean plus three times the standard deviation of the blank). Additional augmentations or modifications can be, for example, employing more powerful laser and/or improved filter set and optics components. With such augmentations, a dose response curve can be obtained in the fg/ml range.

Additionally, a variety of fluid handling systems can be employed in the integrated biosensor and bioprocessor of the invention. For example, pumps and valves can be utilized to automate sample preparation step. An example of a pumping system is described below. Other examples include systems based on restriction of the flow channel or on heating the reagents. However, use of the latter two systems can require extra steps or modifications because of possible clogging of the instrument or denaturing of samples, respectively. Given the teachings and guidance provided herein those skilled in the art will know what precautionary steps can be performed to maintain efficiency of the integrated biosensor of the invention when restriction or temperature is used as a means for moving fluids.

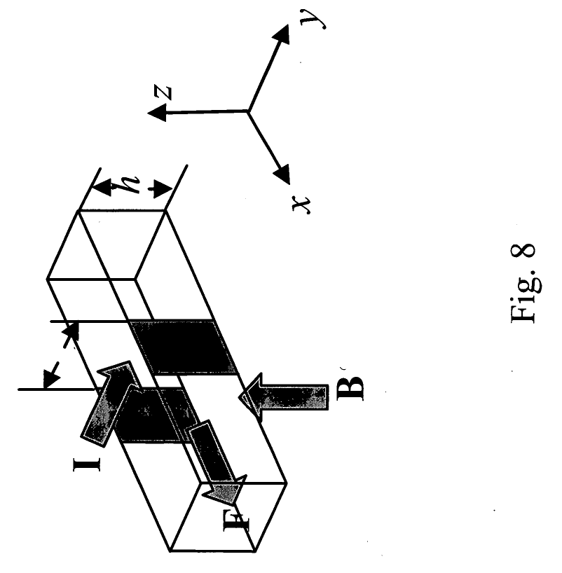

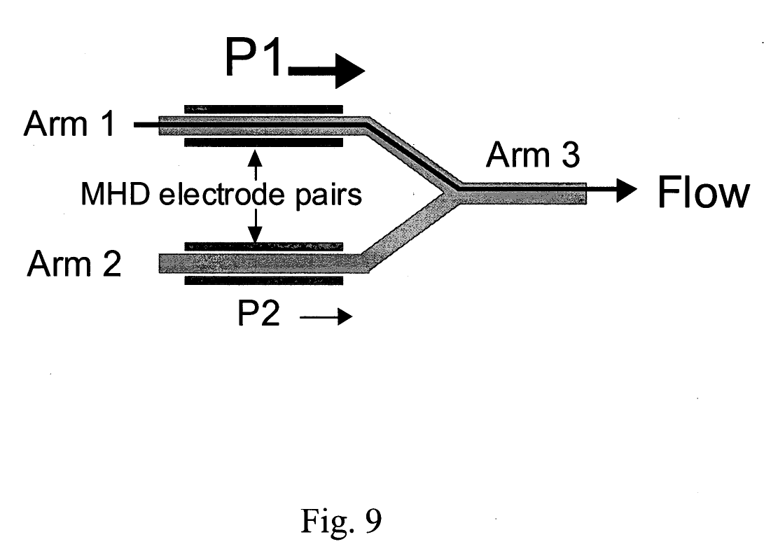

Another type of fluid pumping or handling system includes an AC magnetohydroydynamic (MHD) micropump and MHD micro-fluidic switch. The workings of this type of fluid handling system is well known in the art and can be found described in, for example, Lee, A. P. & Lemoff, A. V. Magnetohydrodynamic (MHD) Devices for Multi-Functional Integrated Micro-fluidics, in Lab-On-A-Chip: Chemistry In Miniaturized Synthesis And Analysis Systems, E. Oosterbroek, Editor. Elsevier: Amsterdam, The Netherlands, (2002); Lemoff and Lee, Biomedical Microdevices, accepted, (2002), and Lemoff and Lee, Sensors and Actuators B 63, 178, (2000). Briefly, the pumping mechanism for an MHD micropump results from the Lorentz force produced when an electrical current is applied across a microchannel filled with conducting solution in the presence of a perpendicular magnetic field as shown in FIGS. 8 and 9. The Lorentz force can be produced, for example, using a DC or an AC set-up. In a DC configuration, a DC current is applied across the channel in the presence of a uniform magnetic field from a permanent magnet. In micro-fluidics using a DC set-up, the same electrolytic reaction that enables current conduction also produces gas bubbles that can impede fluid flow and causes electrode degradation. However, using an AC set-up avoids such electrolysis.

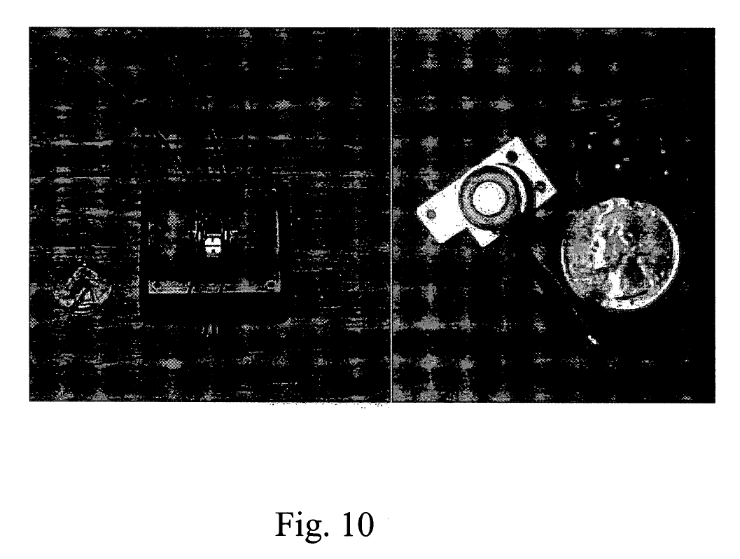

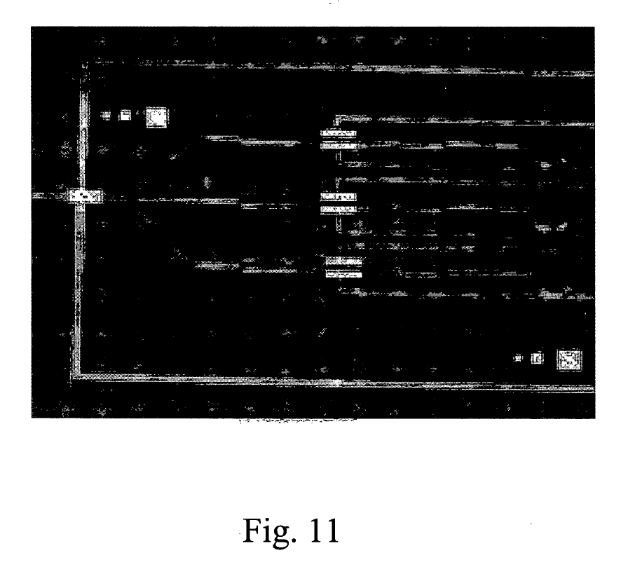

Briefly, in an MHD micropump, an AC electrical current can be used with a perpendicular, synchronous AC magnetic field from an electromagnet. When an AC current of sufficiently high frequency is passed through an electrolytic solution, the chemical reactions are reversed rapidly enough that bubbles essentially do not have a chance to form and no electrode degradation can occur. In this case, the time-averaged Lorentz force not only depends on the current amplitude or the magnetic field amplitude but also depends on the phase of the magnetic field, relative to the electrode current. The ability to control the phase difference enables the control of not only the flow speeds but also the flow direction. At 0° phase, the resulting force is positive and corresponds to a flow in one direction. At 180°, the resulting force is negative and corresponds to flow in the opposite direction. At 90° phase, there is no net flow. The AC current does not damage cells because it has been applied to cell counting applications. FIG. 10 is a photograph image of the packaged MHD micro-fluidic chip as compared with a US quarter that has been implemented with a variety of solutions, including PBS, NaCl, and NaOH, for example. FIG. 11 shows a MHD micro-fluidic circuit implemented with glass-PDMS microfabrication.

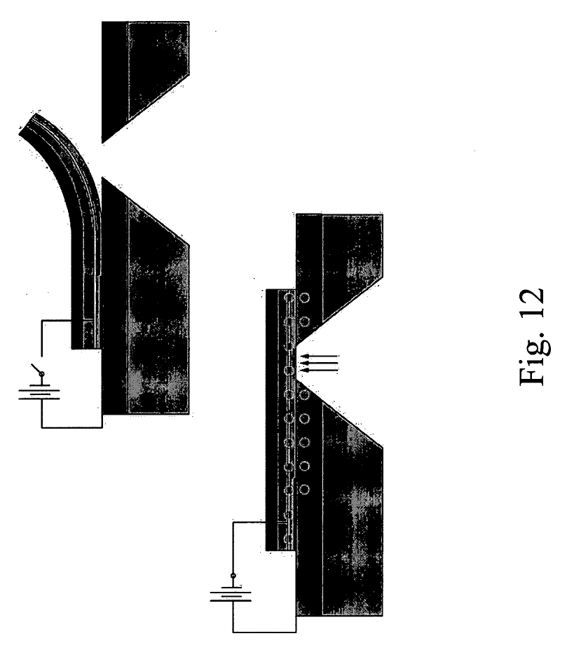

Microvalves can additionally be employed in conjunction with an integrated biosensor and bioprocessor of the invention. Microvalves are particularly useful when performing multi-analyte assays to reduce or prevent cross-contamination from analyte to analyte or from sample to sample. An example of a microvalve applicable for use in the integrated biosensor of the invention is a polyimide microvalve. FIG. 12 shows a schematic drawing of a polyimide microvalve that can be employed to automate sample transport among different bioanalysis components, for example, between a PCR chamber to electrophoresis inlet. Polyimide microvalves are well known in the art and can be found described in, for example, Lee and Trevino, A Low Power, Tight Seal, Polyimide Electrostatic Microvalve, in MEMS Symposium of DSCD, IMECE. Atlanta: ASME. (1996).

The above and other microvalves can be integrated in the integrated biosensor and bioprocessor of the invention as a switch to introduce reagents into the bioanalytical assay regions. Three layers of polyimide thin films can be deposited and patterned by lithography. For example, the top layer can have a higher coefficient of thermal expansion (CTE) than the lower layers, while a thin film metal is sandwiched between the two lower layer polyimides, resulting in a curved up initial state as the composite cantilever is released from the substrate. The metal layer forms a capacitor with the conducting substrate, and a voltage applied generates an electrostatic attractive force between the electrodes. Given the teachings and guidance provided herein, those skilled in the art will understand that the various augmentations and modifications can be incorporated into an integrated biosensor and bioprocessor of the invention to achieve a unitary device that can capture, grow and detect a target analyte.

A flow-through chemical and biological sensor can additionally be employed in the integrated biosensor and bioprocessor of the invention. A flow-through chemical and biological sensor can perform the same function as the integrated biosensors described previously. For example, a flow-through chemical and biological sensor as described below can substitute for a capillary tube in the apparatus as described previously.

Briefly, a flow-though chemical and biological sensor of the present invention can be used, for example, to detect a wide range of biological, biochemical or chemical analytes. The flow-through chemical or biological sensor of the invention also can be used, for example, to detect one or more of many different analytes in a variety of different formats including, for example, serial, parallel or multiplex formats. Analytes to be detected can include, for example, DNA, RNA, proteins, toxins, bacteria, spores, oocysts, cells, cell fragments, viruses, antibodies, polysaccharides, tumor markers, tissue, food, organic and inorganic compounds, that can be present in or placed into a liquid medium such as water, buffer, serum, whole blood, urine, sweat, sputum, saliva, milk, juices, etc. Similarly, analytes in air and solid samples can also be detected using the sensor or methods of the present invention.

Sample preparation can additionally be employed in conjunction with the apparatus and methods of the invention. Those skilled in the art will know which preparatory procedures are useful given the sample and the analyte to be tested. Specific examples of three sample preparations are provided below for illustrative purposes. Briefly, air samples can be prepared, for example, prior to analysis by bubbling air through a liquid, by use of wet wall cyclone aerosol collector or by electrostatic aerosol collector as well as others well known in the art followed by testing the liquid. A solid sample can be prepared, for example, prior to analysis by dissolving the sample in a liquid solution or mixing or homogenizing it in a liquid. A solid sample also can be embedded in a matrix with subsequent processing into a suitable liquid or particulate suspension. Preparation of the matrix that an analyte can be embedded is well known in the art and can differ depending on the matrix and the analyte. For an example, the following procedure can be used to prepare ground beef samples to detect E. coli O157 (see, for example, D. R. DeMarco and D. V. Lim, Detection of Escherichia coli O157:H7 in 10- and 25-gram ground beef samples using an evanescent wave sensor with silica and polystyrene waveguides. J. Food Protection 65, 596-602 (2002). Twenty five gram samples of commercially-purchased ground beef in sterile, plastic conical tubes can be homogenized with twenty-five ml of buffer. The homogenized sample will be centrifuged at 290 RCF for 5 minutes at 4° C. A middle layer containing pathogen can be collected and transferred to a sterile tube, and mixed by vortex. The obtained sample is suitable for use in the sensor and methods of the invention.

Additionally, preparatory procedures suitable for the testing of pathogens in a liquid can additionally include a filtering, concentration or centrifugation step or combinations of these steps. Those skilled in the art will understand given the teachings and guidance provided herein which preparatory step is beneficial to include depending on the nature and quantity of the liquid and sample analyte. For example, it can be beneficial to remove large particles in the liquid, as well as other contents that could interfere with the sensor's operation. To detect low concentrations of pathogens in the liquid, the liquid can be concentrated and the concentrated liquid used for the sensor assay.

For waveguide sensors, the waveguide can be coated with an appropriate molecular recognition species, also referred to herein as an analyte recognition coating or analyte recognition element. Where the capture configuration is in solution phase for initial analyte binding as described previously, the waveguide can be coated with a secondary binding partner. Such coatings, elements or secondary binding partners can include, for example, a protein (e.g., antibody, antibiotic, an antigen target for an antibody analyte, cell receptor protein, avidin), a nuclear acid or related to nucleic acid (e.g., oligonucleotide, DNA, cDNA and RNA), polysaccharide, monosaccharide, oligosaccharide, aptamers, ribozymes, enzymes, ligands, cell and cell fragment as well as other biological particles. This molecular recognition species will serve to capture the analyte on the waveguide when the assay is performed. The prepared waveguides can be stored until use when the assay is performed or used immediately after functionalization with a recognition species.

The presence of the analyte can be detected, for example, via electromagnetic radiation. All wavelengths within the electromagnetic spectrum that can transmit in the waveguide can be used to specifically detect an analyte using, for example, an emission detection reagent. Useful detection spectrum includes, for example, the visible spectrum, emitted by a fluorescent, phosphorescent or luminescent detection reagent or label attached to, for example, a secondary molecular recognition species and infrared spectrum. The labeled secondary molecular recognition species can be any labeled species that recognize and bind to the captured analyte or to the complex formed by the analyte bound by the primary molecular recognition species such as an analyte recognition coating or element.

In addition, binding and detection methods and other than those described above and below are known in the art. Such other methods and formats are equally applicable in the sensor apparatus or methods of the invention. The apparatus and methods of the invention include the capture of an analyte by an analyte recognition coating or element. Capture can be accomplished by, for example, any affinity binding means that is specific for the analyte of interest. For example, binding formats applicable for use in the invention include direct binding of the analyte by the analyte recognition element or indirect binding by, for example, an intermediate affinity binding reagent. Binding and detection also can be performed in a sandwich format in which the analyte is bound between an analyte recognition coating and a detection reagent. As described previously, capture of the analyte can be via solution or solid phase configurations with the analyte recognition coating or element and then bound by a secondary binding partner to a waveguide. Other formats well known to those skilled in the art also can be employed in the apparatus and methods of the invention.

Further, the apparatus and methods of the invention include the detection of bound analyte by an emission detection reagent. Various emission detection reagents well known to those skilled in the art can be employed in the sensor apparatus and methods of the invention. Such emission detection reagents include, for example, luminescent, fluorescent and phosphorescent emission detection reagents, all of which can be employed with any of the various binding methods or formats described herein or well known to those skilled in the art. Additionally, such detection reagents can be employed in modes that include direct binding to an analyte or an analyte bound to a recognition coating. Alternatively, emission detection reagents can be employed in modes that include indirect binding to an analyte or an analyte bound to a recognition coating. Further, the binding and detection methods and formats for analyzing also can include methods such as FRET (fluorescence resonance energy transfer) where an optical signal is generated following a change in proximity of the fluorescent detection reagent from the quencher following binding of analyte. A change in proximity can include, for example, a release of the emission detection reagent such as by cleavage with a protease analyte, or a change in conformation due to analyte binding.

The binding or detection methods or formats are well known to those skilled in the art and can be employed in the apparatus of the invention. Similarly, other well known binding or detection methods or binding or detection formats also can be employed in the apparatus or methods of the invention. Given the teachings and guidance provided herein, those skilled in the art will understand that any of the various binding or detection methods or formats well known in the art can be used in conjunction with the methods or formats described herein. Similarly, given the teachings and guidance provided herein, those skilled in the art will understand that the various binding or detection methods or formats can be substituted or used in various combinations with the methods and formats exemplified herein.

The invention provides a mixing flow apparatus. The mixing flow apparatus consists of a waveguide and a mixing flow chamber; the waveguide having an appropriate index of refraction material for propagation of a radiation signal, and the mixing flow chamber having a body forming a flow chamber with an inlet, an outlet, a radiation transmissive wall and a surface positioned to disrupt flow regularity of a sample fluid, the body of the mixing flow chamber surrounding at least a portion of the waveguide, wherein constituents of a sample fluid entering the inlet are mixed by disruption of sample fluid flow regularity prior to discharge at the outlet. The mixing flow chamber surface can be positioned to disrupt flow regularity by, for example, structural or spatial configurations. The mixing flow chamber surface also can be positioned to disrupt flow regularity by, for example, inclusion of specific shapes or being activatable. Shapes include, for example, physical protrusions as well orifices that allow injection of gases, vapors and the like that disrupt flow directly or that generate bubbles which disrupt flow.

Also provided is a detection apparatus. The detection apparatus consists of a waveguide, a mixing flow chamber and a radiation detector; the waveguide having an appropriate index of refraction material for propagation of a radiation signal; the mixing flow chamber having a body forming a flow chamber with an inlet, an outlet, a radiation transmissive wall and a surface positioned to disrupt flow regularity of a sample fluid, the body of the mixing flow chamber surrounding at least a portion of the waveguide, wherein constituents of a sample fluid entering the inlet are mixed by disruption of sample fluid flow regularity prior to discharge at the outlet, and the radiation detector being disposed facing the direction of oncoming propagated signal from the waveguide. The mixing flow chamber surface can be positioned to disrupt flow regularity by, for example, structural or spatial configurations. The mixing flow chamber surface also can be positioned to disrupt flow regularity by, for example, inclusion of specific shapes or being activatable.

A mixing flow apparatus of the invention consists of a waveguide and a mixing flow chamber. The apparatus can be used alone as a mixing device or for the detection of analytes with inherent optical emissions. In the latter example, the mixing flow apparatus can be coupled, for example, to a detector for measuring analyte emissions. Alternatively, qualitative observation can be used when the emission intensity is sufficiently strong. Additionally, the mixing flow apparatus can be combined, for example, with a radiation source or a detection device to produce a sensor. The mixing flow chamber or cartridge can be a stand alone cartridge or part of a larger cartridge. Specific examples of a mixing flow chamber or cartridge include those shown in the figures and described further below as well as micro chips and microfluidic chips. The various embodiments of the mixing flow apparatus or the apparatus combined with other sensor hardware for detection of incident radiation are exemplified below.

For example, radiation or detection hardware of the sensor can include an instrument to control and perform an assay and a chamber or vessel in which the assay takes place including, for example, any stirring or mixing of the reagents and analyte that results in the capture and identification of the analyte. This chamber or vessel can be affixed to or detachable from the instrument and can be a reusable, rechargeable, or disposable cartridge. This chamber or vessel is also referred hereafter in its various forms as a mixing flow cartridge. A mixing flow cartridge consists of at least one mixing flow chamber and at least one waveguide. The mixing flow cartridge can be re-usable for a number of times. Reuse of the mixing flow cartridge is particularly useful in instances where initial test result are negative. The sensor instrument also can include, for example, radiation illumination member(s), radiation detector member(s) (such as photodiodes, CCDs, photomultiplier tubes (PMTs), position sensitive PMTs, CMOS arrays, spectrometers, etc.), a fluid handling member (such as pumps, valves, switches, meters, etc), electronics member (such as circuits, displays, timers, etc.) and software programs.

The fluid flow in the mixing flow chamber is designed to improve the capture of the analyte by the waveguide by passively or actively stirring the sample to enable constituents of the sample to come in contact with the analyte capture surface. Exemplary embodiments of passive mixing of the analyte include, for example, an the inclusion of an undulating shape of the mixing flow chamber wall. This undulating shape can cause the fluid in the mixing flow chamber to move about in a turbulent manner as it flows from inlet to the outlet. A static waveguide inside the body of the mixing flow chamber also can act as a mixing element, creating turbulence in the sample. Additionally, the waveguide inside the body of the mixing flow chamber can be, for example, attached to the mixing flow chamber on one end and the other end is allowed to move.

Exemplary embodiments of active mixing of the analyte. can include, for example, unattached or attached members of similar or different material placed inside the mixing flow chamber. These members can be allowed to move inside the body of the mixing flow chamber and also can be actuated by mechanical, thermal, electrical or magnetic forces. Additionally, for example, sample can be pumped into a mixing flow chamber from different inlets and pumped out of the mixing flow chamber from different outlets at the same or at different times. The flow direction can be periodically reversed. The pumping speed also can be modulated.

With respect to the physical structure of a mixing flow chamber or cartridge, in one specific embodiment a mixing flow chamber can consist of a fluid sample mixing flow chamber having a body, at least one waveguide member. As stated previously, the waveguide can be, for example, connected to the mixing flow chamber. Alternatively, the mixing flow chamber also can include, for example, a waveguide not connected to the mixing flow chamber or a mixing flow chamber can include multiple waveguides, all connected to the mixing flow chamber, some connected and some unconnected to the mixing flow chamber or all unconnected to the mixing flow chamber. A mixing flow chamber also can include, for example, chambers containing reagents and a chamber to be filled with sample fluid. This embodiment of a mixing chamber includes a first end and a second end, side walls, a clear top surface, a bottom surface, at least one inlet, and at least one outlet. The body of the chamber extends outward from the waveguide member and is spaced therefrom so as to allow a fluid to flow between the inlet and the outlet.

The waveguide member can be coated with analyte capture elements. Assays can be performed to capture the analyte, and the analyte can be tagged with an emission detection reagent or labels. Excitation light impinges on the emission detection reagent to cause it to produce light. The waveguide, capturing a portion of the emission light along with some excitation light and propagating them to one end. The light emerges from the waveguide and passes through lens, filters or grating system before detection by an optical or infrared detector.

The body of the mixing flow chamber can consist of, for example, one clear element through which the excitation light enters the mixing flow chamber. This clear element can have flat top and bottom surfaces to provide uniform illumination along the long direction. This clear element can have curved surfaces to focus the excitation light on to the waveguide. This clear element can also serve as a waveguide. The ends of this clear element can be coated with reflective material. Some parts of the sides or other areas of this clear element can be coated with reflective and/or light absorbing material. Additionally, when the clear element of the body is not the waveguide, some parts of this clear element can be coated with light absorbing material.

One or more sides of the mixing flow chamber can have undulating surfaces that vary in the long direction and that serve to stir the fluid as it flows through the mixing flow chamber, while other portions of the surface can be smooth in the long direction. The undulating shaped surfaces can be on one side, two sides or all sides of the mixing flow chamber. Some parts of the undulating and smooth surfaces can have light absorbing properties. Some parts of the undulating and smooth surfaces can have reflective properties. Some parts of the surface can be clear. Undulating walls can have any shape, as long as they function to mix the sample fluid and minimize fluid trapping. An undulating shape can be periodic in the long direction, for example.

In another specific embodiment of the invention, no surface of the mixing flow chamber has undulating walls. The surfaces of the mixing flow chamber are smooth and can be flat or have uniform curvature. The mixing can be performed, for example, by flow over stationary waveguides inside the mixing flow chamber, by waveguide motion inside the mixing flow chamber actuated externally by the waveguide motion inducedby the flow over the waveguide, by motion of embedded elements inside the mixing flow chamber actuated externally by electric or magnetic forces, or by temporally or persistent modulated pumping action of the fluid.

The material of the mixing flow chamber wall can be different or the same as the waveguide. The mixing flow chamber can have at least one inlet and at least one outlet.

In another specific embodiment of the invention, the mixing flow chamber can have multiple mixing flow chambers each with at least one each of waveguide, inlet and outlet.

The body of the mixing flow chamber can be made of any material compatible with the sample fluid and assay reagents. Generally, the body of the mixing flow chamber is made of a polymer that can be manufactured, for example, by injection molding, such as polymethylmethacrylate, polycarbonate, or polystyrene. The body of the mixing flow chamber forms a tight seal to prevent loss of sample fluid. The body of the mixing flow chamber can be either rigid or elastic. Materials for all parts of the body of the mixing flow chamber should be compatible with the analyte and the assay reagents. Given the teachings and guidance provided herein, those skilled in the art will know, or can readily determine those material having compatibility with the analyte binding and detection methods described herein.

With regard to the waveguide, radiation from emission detection reagent attached to a higher index of refraction waveguide than its surroundings is partially radiated into the waveguide and partially into the surroundings. See Jin Au Kong, Electromagnetic Wave Theory (First Edition, John Wiley & Sons, Inc., New York, 1975; Second Edition, John Wiley & Sons, Inc., New York, 1990) and Cha-Mei Tang, IEEE Transactions On Antenna And Propagation, AP-27 (5), 665-670 (1979). In the context of the apparatuses of the invention, the higher index of refraction material for propagation of an emitted signal is referred to herein as a waveguide. The waveguide provides the ability to direct the emitted signal into the waveguide and to the detector.

The waveguide can be, for example, one of the elements that constitute the sides of the mixing flow chamber, or it can be suspended in the middle of the mixing flow chamber. The waveguide can have any shape. Generally, the waveguide is elongated in one dimension. The surface of the waveguide should be optically smooth to provide low loss of the optical signal.

The shape of the cross section can vary so as long as it remains a medium that can propagate an optical signal for at least a short distance, such as the distance from signal emission along the waveguide to the exit end of the waveguide to the detector. This distance also can include the entire length of a waveguide. For example, some of cross sectional shapes can be circles, ovals, ellipses, squares, rectangles, diamonds, polygons, rings, or other shapes that can propagate emitted radiation signal from captured analyte to a detector. Accordingly, a waveguide does not need to be straight in the long direction. It can have sections that include arcs, loops, oscillations, so long as it facilitates propagation of an emitted radiation signal from captured analyte to a detector.

A waveguide can be made of any material, for example, that transmits light at both the excitation wavelength and the signal emission wavelength. A waveguide can consist of a single material or consist of a composite of two or more different materials. The composition of waveguide materials can vary, for example, in the long direction as well as in the transverse direction. Different sections can have different materials. Generally, the waveguide can be an inorganic glass or a solid such as a polymer (e.g., a plastic such as polystyrene). The waveguide can have multimode or single mode optical properties.

The waveguide can be coated with reflective material on the surfaces of some of the transverse direction, or on one end of the waveguide. The reflective coating can be any material that reflects light at the excitation wavelength at some parts of the waveguide, and the coating can reflect light at the emitted signal wavelength at some parts of the waveguide, or both. The reflective coating can also be any material that reflects both the excitation and emission wavelength. Generally, a reflective coating includes a reflective metal, such as aluminum, silver, gold, chromium, platinum, rhodium, or mixtures thereof. More often, a reflective metal is aluminum, silver, or gold. Additionally, the reflective coating can consist of multiple layers, such as dichroic mirror, or reflective material and bonding material.

The reflective coating can be applied to the surface of the waveguide in any manner well known in the art for such procedures. Vacuum evaporation deposition of the reflective coating on glass and plastic substrates is one exemplary method. Lithography patterning technique also can be used. Electroless deposition is yet a further exemplary method.

Specific examples of waveguides include a round optical fiber having transmission properties. The round optical fiber can be coated on one side with reflective coating. When used as a waveguide, one laser source will be able to provide improved uniformity of illumination. Rectangular optical fiber coated with reflective material on two opposite sides can provide uniform illumination and good signal transmission. Further, for example, capillary tubes can be used both as a mixing flow chamber and waveguide. Capillary tubes can be coated with reflecting material on a portion of the exterior surface to improve the illumination of the analyte capture surface inside the capillary tube.

The waveguide shape and features can vary along the long axis. Some common changes in features are the dimensions of the waveguide, abrupt transition in shape, or smooth transition in shape or changes in coatings. For example, the cross sectional size can vary from a circle of larger diameter to a smaller diameter. For example, the cross sectional shape can vary from a polygon to a circle.

In addition, the present invention allows the attachment to the waveguide of other optical elements. Such other optical elements can include, for example, lenses or optical filters.

The mixing flow or detection apparatuses of the invention can be used for single or multiple analyte detection. For example, the apparatuses and methods of the invention allow for detection of a single analyte or the simultaneous detection a multiple analytes on a single waveguide or on multiple waveguides, independently or simultaneously. The optically clear surface of the waveguide inside the mixing flow cartridge serves to capture the analyte to be measured. The amount of surface area needed for detection depends on the desired detectable concentration level. A range of the analyte capture surface area can vary from 0.01 μm² to many cm².

In one specific embodiment, multiple analyte detection can be achieved by patterning the waveguide in sections, each with a different analyte capture surfaces sensitive to a specific analyte.

In another specific embodiment, the waveguide surface is simultaneously coated with different analyte capture chemical elements. As sample flows through the mixing flow chamber, multiple analytes in question can be simultaneously captured along the whole length of the waveguide.

In a further specific embodiment, multiple analyte detection can be achieved with a sensor having multiple mixing flow chambers. In this embodiment, each of these mixing flow chambers contains at least one waveguide that is coated with an analyte capture surface.

In still a further specific embodiment, more than one waveguide can be used to detect the same analyte. This method can be used to increase the analyte capture surface area or to increase the mixing of the fluid.

A mixing flow apparatus can be configured as a detection or as a signal detection apparatus. Such detection or signal detection apparatuses can consists of, for example, (1) one or more light sources to illuminate (excite) the emission detection reagent to produce an emitted signal light, (2) optical system, (3) a detector system to capture the emitted signal light, (4) fluid handling system, (5) data acquisition, signal analysis and data output. The excitation light source impinges on the optical labels not by internal route through the waveguide by evanescent method, but by external route independently outside the waveguide. For detection of analytes having inherent optical properties, such as chemiluminescent labels, the illumination source can be omitted or unused in the apparatus.

One component of the instrument is the radiation illumination member, consisting of light source(s) and optics. For some applications, such as colloidal gold and silver, the excitation light source can be a broad-spectrum source, while in other applications, the excitation light source can be a narrow spectrum. Some waveguides can be better illuminated using multiple light sources. In some multiple-analyte applications, for example, with more than one fluorescent label on the same waveguide, some labels can require one or more narrow band excitation light sources, while other labels, such as quantum dots, can require a single broadband excitation light source for all emission wavelengths. Lenses, filters, and other optical devices can be needed to achieve the desirable illumination.

Excitation light source in the present invention can use any light source using any of various methods well know in the art. Exemplary sources include, lasers, light emitting diodes (LEDs) and broadband light sources.

Briefly, light from a laser has the property of coherence and potentially high power, narrow wavelength band beam that can be turned into a wide parallel beam, a cone beam or a fan beam with lenses. Coherence and high power provide larger power density. Narrow band is desirable for organic dyes. Any kind of laser can be used in the apparatuses and methods of the invention. Diode lasers are commonly available, compact and relative low cost.

Light Emitting Diodes or (LEDs) produce incoherent light, lower power light. LEDs are inexpensive and compact and therefore beneficial for some applications. Alternatively, an addressable multiple-element array of optical sources, such as LEDs, can be used to sequentially probe each patterned region of the waveguide. This multiple element array of optical sources provides a particularly low cost technique, having the advantage of no moving parts, and providing more flexibility than stepped or oscillated excitation light, because LEDs or groups of LEDs would be addressable in any arbitrary temporal or spatial sequence.

Broadband incoherent light sources including, for example, incandescent lamps xenon lamps, mercury lamps and arc lamps also are useful in the apparatuses of the invention. For example, broadband ultra violet (UV) sources can be useful for illuminating quantum dot labels.

A wide variety of excitation light source configurations are possible for using in the radiation illumination member. The selection among alternatives will depend, in part, on the type of recognition element patterning on the waveguide.

In this invention, the temporal mode of radiation illumination and radiation detection can include, for example, a variety of methods and variations. Specific examples of such modes include instantaneous signal, time averaged instantaneous signal, time integrated partial signal, time integrated continuous whole signal, frequency modulated signal, or other variations or combinations thereof. The temporal mode of illumination and detection is related to the method of spatial illumination of the excitation light, the flubrescent labels, the waveguide geometry, the number of analytes to be detected, the concentration level of the analyte, and the desired sensitivity of the detection.

Excitation light source can impinge on the emission detection reagent of one or more analytes during the entire period of detection of each analyte. The excitation light source can be modulated or “chopped” as a means to eliminate interference from ambient light. Demodulation of the resulting emitted signal, such as with a lock-in amplifier, can then reduce background interference. Such modulation can not be required, if ambient light is eliminated by proper optical isolation or shielding.

One method of illumination is for the excitation light source to emanate from a wide or diffused area, and to illuminate the entire analyte capture surface of the waveguide(s) from one or more directions. Advantages of this unfocused or diffused area of illumination method include: (1) it would illuminate substantially the entire analyte sensing area on one or more waveguides, (2) it minimizes alignment procedures, since the illumination areas is larger than the waveguide areas.

An alternate method of illumination is for the excitation light source to emanate from a point source or to be focused to a point source, and thence illuminate the analyte-sensing area on the waveguide. This method of illumination can use focused or collimated light from a laser or other source and can illuminate a portion of the waveguide. Advantages of this focused or point source method include: (1) greater excitation light intensity; (2) ability to control and manipulate the angular distribution of the excitation light; (3) the potential to use high sensitivity, background- and noise-rejecting electronic signal processing methods (e.g., modulation and demodulation); and (4) possibility to reduce cross talk from other analytes and nearby waveguides.

One or more excitation light sources can be used sequentially or simultaneously to provide different illumination wavelengths and/or to provide different spatial and temporal coverage. The angle of incidence of the excitation light can be perpendicular to the incident surface of the waveguide, perpendicular to the length of the waveguide, or at one or more angles in relation to the surface of the waveguide. The optimal angle of illumination can be selected so as to reduce the background noise resulting from excitation light or to enhance any other desirable characteristics of the sensor. The excitation light can be collimated, non-collimated, point source, multiple point sources, diffused source or broad area unfocused source. The angle of illumination is not limited to excitation perpendicular to the surface of the waveguide.

An optimal angle of illumination is dependent on the size and shape of the waveguide and the desired detection limit. Long waveguides can reduce collected excitation light at the detector because each time the excitation light reflects on a boundary of the waveguide, part of the excitation light is lost due to transmission out of the waveguide. The loss is largest at the perpendicular angle. The excitation light can also be in the form of evanescent wave with the light input at the end of the optical fiber.

A radiation detection device can be placed where light exits from the waveguide in order to detect the signal produced by the label(s). The detector assembly can consist of an optical system in addition to the radiation detection device.

Emission signals produced by the labels can be detected by a variety of different detectors, such as photodiodes, one-dimensional charge-coupled device (CCD) arrays, two-dimensional CCD arrays, photo-multiplier tubes (PMT), position sensitive PMTs, CMOS image arrays, spectrometers, etc. The PMT should preferably be chosen to have maximum sensitivity in the region of radiation of the labels and should preferably be provided with a filter blocking the light emitted by the source radiation. One or more detectors can be used.

The emission signal produced by the labels can be detected (1) as a total power independent of the frequency or position, (2) as a total power as a function of position independent of the frequency, (3) as power in the frequency spectrum independent of position and (4) as power as a function of position and frequency.

The emission signal produced by the labels can be amplified electronically or using photomultiplier tubes (PMTs). The emission signal produced by the labels can be detected as instantaneous, time averaged or time integrated power. For labels such as quantum dots, which can remain photo stable after exposure to long periods of excitation light sources as compared to organic dyes, integration of the signal over long period of time becomes possible and can be used to improve the sensitivity.

Optics are used to minimize the excitation light entering the detector. Some examples of the embodiments are as follows: (1) use of wavelength dependent filters, (2) use of a grating outside the waveguide to spread the light into a spectrum of wavelength and use only the signal from the emission light wavelength, and/or (3) use of gratings or absorbent coatings on the waveguide surfaces to allow the transmission of emission light and prevent the transmission of excitation light from the waveguide to the detector.

Various lenses, mirrors, and optical filters can be placed between the waveguide and the detector. For example, a linear lens array in registration with the waveguides can be used. Other options include the use of a pair of linear Gradient-Index (GRIN) lens arrays configured to provide a quasi-collimated region between the arrays for insertion of an interference filter, and an array of cylindrical lenses. Alternatively, optical filters can be directly butt-coupled to the waveguide or to the detector, or both.

The apparatuses of the invention can be automated to include a fluid handling member, which consists of valves, pumps, switches and reagent chambers. The sensor can be constructed with valves, pumps, switches, and reagent chambers as part of the instrument using conventional off-the-shelf components, or some or all these elements can be constructed as part of the mixing flow cartridge.

Fluid flow can be achieved manually with a syringe or other vacuum or pressure device, or automated using a pneumatic, peristaltic, or microfabricated pumps designed to move the solutions inside the mixing flow chamber. A non-optical filter can be placed at the inlet of the mixing flow chamber in order to prevent undesirable particles from entering the mixing flow chamber. The sample can be recirculated through the mixing flow chamber to increase the chance of capture. Samples can enter from more than one inlet and exit from more than one outlet. The flow into each inlet and out of each outlet can individually and temporally modulated. The flow direction can be reversed, such that the inlet can become the outlet for certain periods of time.

The types of assays that can be performed include, for example, (1) a competitive assay (wherein labeled and unlabeled analyte compete for open binding sites), (2) a displacement assay (wherein unlabeled sample analyte dissociates bound labeled analyte or molecular recognition species on a waveguide that has been previously coated with bound labeled analyte), (3) a sandwich assay (wherein sample analyte binds to a primary molecular recognition species on the waveguide surface, and a labeled secondary molecular species binds to the immobilized analyte or the immobilized analyte/primary molecular species complex), nucleic acid hybridization assay, (4) Fluorescence Resonance Energy Transfer (FRET) assay (wherein sample analyte causes a change in a recognition species bound on the waveguide to produce a fluorescent signal), (5) chemiluminescence assay (wherein sample analyte causes a chemical or other type of reaction on a recognition species bound on the waveguide to produce a luminescent signal), or any other type of bioaffinity or chemical affinity assay that produces a detectable signal.

A wide variety of analyte recognition elements and methods for attaching them on the waveguides can be used with the present invention. One common feature among the various assays is that the surface of the waveguide is coated with an analyte recognition element. Analyte recognition on the waveguide surface can also be accomplished by means other than the attachment of a molecular recognition species. For example, the analyte capture surface can be formed by coating the waveguide surface with a binding material, such as avidin, a doped or undoped polymer, or sol-gel that exhibits a differential optical response upon exposure to the analyte or an analyte complex including, for example, a combination with an additional label or labels. An example of one such non-biomolecular recognition species is provided in MacCraith, Sensors & Actuators 29(1-3), 51-57 (1995).

Regardless of how analyte recognition is achieved, an emission detection reagent is typically used to generate an optical signal to indicate the presence or absence of the analyte. If a sandwich assay is desired, the labeled secondary molecular recognition species can be any labeled species that recognizes a molecular binding site on the analyte capture complex, immobilized analyte or the immobilized molecular recognition species/bound analyte complex.

In the present invention, typical methods for attaching molecular recognition species to surfaces include covalent binding, physisorption, biotin-avidin binding (such as described in Bhatia et al., Use of Thiol-Terminal Silanes and Heterobifunctional Crosslinkers for Immobilization of Antibodies on Silica Surfaces, Anal. Biochem. 178 (2): 408-413, May 1 (1989); Rowe et al., An array Immunosensor for Simultaneous Detection of Clinical Analytes, Anal. Chem. 71 (2), 433-439 Jan. 15, 1999; Conrad et al., U.S. Pat. No. 5,736,257; Conrad et al., SPIE, 2978, 12-27 (1997); Wadkins et al., Biosensors & Bioelectronics 13 (3-2): 407-415 (1998); Martin et al., Micro Total Analysis Systems (Kluwer Academic Publishers, Netherlands, 1998 p. 27), or modification of the surface with thio-terminated silane/heterobifunctional crosslinker as in Eigler et al. [sic], U.S. Pat. No. 5,077,210 issued Dec. 31, 1991, or the use of APTES/NHS-Maleimide bifunctional linker/Thiol modified polyethylene glycol (see Soon Jin Oh et al., Langmuir 18, 1764-69 (2002)). The immobilization of molecular recognition species to the waveguide can also use polyamidoamine (PAMAM) dendrimers (See R. Yin et al., Dendrimer-Based Alert Ticket: A Novel-Biodevice for Bio-Agent Detection, Polymeric Materials: Science & Engineering 84, 856-857 (2001)). Attachment of analyte recognition reagent can also be achieved by photolithographic method. Alternatively, attachment of molecular recognition species to the waveguide surface can use commercial products such as dendrimer based self assembled monolayer (SensoPath Technologies, Inc., Boseman, Mont.).

Furthermore, in the present invention fluorescent dyes, fluorescent nanoparticles, quantum dots, colloidal gold, colloidal metal plasmon resonant particles, Fluorescence Resonance Energy Transfer (FRET), chemiluminescence and other fluorescent sources can be used to produce the optical signal produced by the capture complex or the analyte/capture complex on the analyte capture surface of the waveguide. In other words, the present invention is not limited by the source or type of assay components.

In one embodiment of the invention, the mixing flow cartridge, containing the waveguide coated with the molecular recognition species, can be stored for a period of time before being used. In another embodiment of the invention, the mixing flow cartridge, containing the waveguide coated with the molecular recognition species and an appropriate labeled or unlabeled analyte/molecular recognition species, can be stored for a period/of time before being used in a displacement assay.

Finally, it should be kept in mind that the waveguides on which molecular recognition coating, for example, can be used more than once. Thus, after detection and analysis, the waveguide can be exposed to an appropriate chemical, biological, or optical, or other treatment as known in the art that is capable of removing the analyte or otherwise restoring the original analyte-sensing properties of the molecular recognition species.

Molecular recognition on the analyte capture surface can also be accomplished by means other than the attachment of a molecular recognition species. For example, the analyte capture surface can be formed by coating a surface of the waveguide with avidin, a doped or undoped polymer or sol-gel that exhibits a differential optical response upon exposure to the analyte or the analyte in combination with an additional label or labels. An example of one such non biomolecular recognition species is provided in MacCraith, B D., Sensors and Actuators B., 29 (1-3): 51-57 October 1995, the entirety of which is incorporated herein for all purposes. The analyte capture surface of the waveguide can be prepared, for example, after the complete construction of the mixing flow cartridge or prepared before the final assembly.

Generally, the space between waveguide member and mixing flow chamber walls can have a dimension of few tens of microns to a few millimeters. The waveguide can have a cross sectional dimension of few microns to few millimeters and have a long dimension of few hundreds of microns to tens of centimeters.

One or more combinations of the sample flow can be employed. For example, single pass, where the sample enters the inlet and exits from the outlet can be employed. Alternatively, recirculating flow, where the sample enters the inlet and exits from the outlet and this process is repeated, improving the percentage of capture over single pass also can be employed. Alternatively, pulsed flow, where for example, the sample flows enters and exits the mixing flow chamber at different velocities at different times creating mixing followed by incubation also can be employed. Additionally, reversible flow, where the sample flows in one direction and the direction reverses so that the inlet becomes outlet and outlet becomes inlet, a useful method when the waveguide layout is not symmetric to the inlet and outlet.

Multiple inlets and outlets can be utilized, including more than one inlet and/or more than one outlet. This configuration can provide a desirable distribution of fluid flow and higher flow rate. Different entrances and exits also can be utilized where, for example, a sample enters different inlets at different times and exits different outlets at different times. Further, multi-analyte testing also can be performed in the apparatuses of the invention. In this embodiment, the same sample can be passed over all the waveguides, each of which can be detecting for a different analyte.

The sensor, in one aspect of the present invention, allows for manual or automated detection of analytes. The instrument format can be a portable kit, a bench top instrument or large high throughput processing systems that can be used to detect and quantify a variety of hazardous substances in numerous sample matrices. The instrument can be used in different types of environments. It allows for rapid and accurate detection of any sort of analyte present in food, water, soil extracts, air extracts, and clinical fluids.

In the following description, in order to facilitate a thorough understanding of the invention and for purposes of explanation and not limitation, specific details are set forth, such as a particular geometry of the sensor. The invention can be practiced, however, in other embodiments that depart from these specific details.

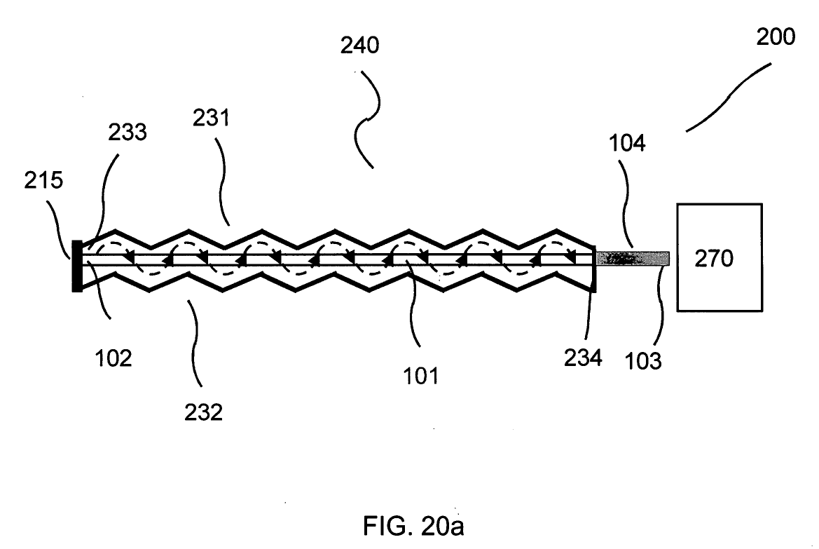

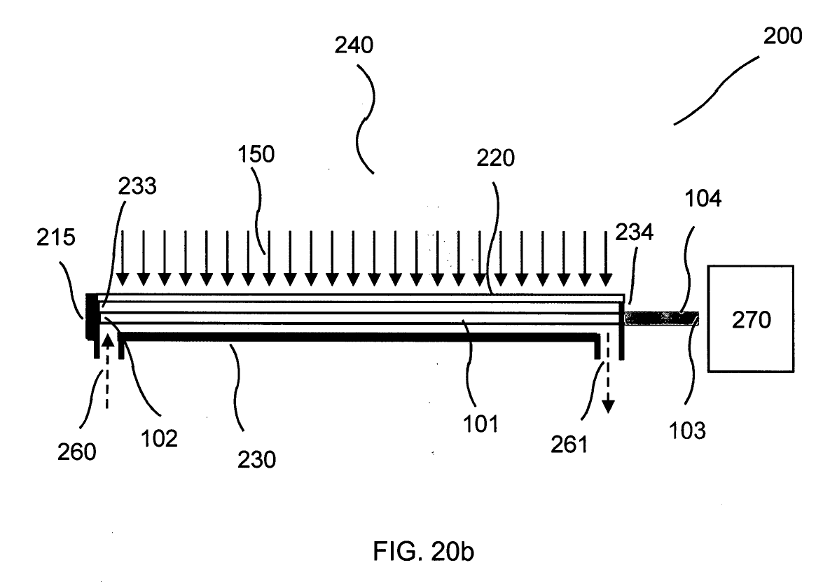

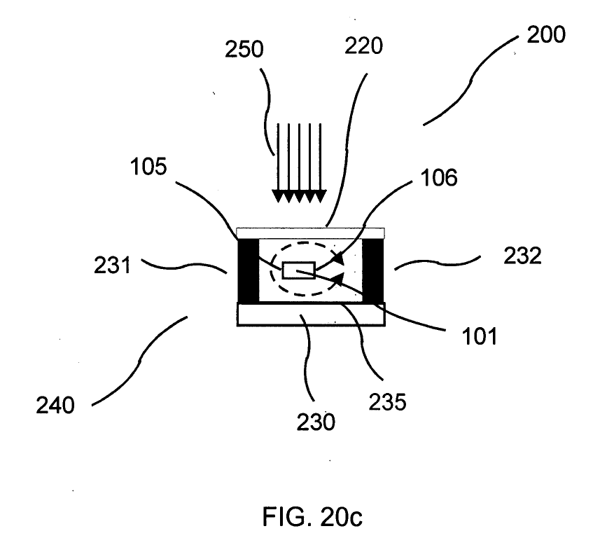

FIGS. 20a-c show the top view, side view and end view of a mixing flow-through sensor according to one embodiment of the invention, respectively. Sensing system 200 consists of a mixing flow chamber 240, waveguide member 101 on which is attached the analyte capture surface, and the detector systems member 270.

In the embodiment shown in FIG. 20a, waveguide member 101 is an elongated member, adapted to propagate along its length the collected radiation. The waveguide member 101 passes through mixing flow chamber 240, so as to expose substantially all of the waveguide surface to the sample, leaving first end 102 and second end 103 of the waveguide unobscured. A reflective surface 215 can be placed at the end of the waveguide 102. More particularly, mixing flow chamber 240 consists of elongated side bodies 231 and 232 that extends outward from waveguide member 101 and is constructed and arranged to house a portion of the waveguide. The mixing flow chamber 240 also consists of first end 233 and second end 234, and the waveguide member 101 is attached at least to the second end 234. The emission signal exits from the waveguide end 103 and enters the detector member 270.

FIG. 20b shows the side view of the mixing flow chamber 240 and further consists of radiation transmissive surface 220 allowing the excitation light to propagate to the analyte capture surface of the waveguide. The lower border 230 can be clear, black or any other color, or coated with reflective or absorbent material. The mixing flow chamber includes an inlet 260 and an outlet 261 to allow a fluid solution to flow inside the mixing flow chamber between inlet 260 and outlet 261. In another embodiment, the position of inlet 260 and outlet 261 can be reversed. The excitation light 250 is directly incident on the waveguide surface.

FIG. 20c shows the end view of the sensing system 200, including transparent top boundary 220, the side walls 231 and 232, the bottom wall 230, the waveguide 101 and incident radiation 250.

The walls 231 and 232, as shown in FIG. 20a, are undulated so that they force the liquid sample to flow from one side of the mixing flow chamber to the other side and to go around the waveguide, as shown in FIG. 20c. The waveguide acts as a mixing stick as indicated in FIG. 20c. Specifically, the shape of the walls and the waveguide 101 prevents the flow from being laminar, and allows all of the analyte in the sample to have a chance to come in contact with the analyte capture surface on the waveguide. The motion of the sample to the first order is indicated by the dashed curves in FIGS. 20a and 20c. Depending on the characteristics of the fluid and the flowing conditions, a turbulence regime can be established inside part of the mixing flow chamber. As a result, the interactions between the constitutive elements of the fluid and the analyte capture surface of the waveguide are significantly enhanced. It follows that the mechanical interactions between the elements of the fluid and the surface of waveguide member 101 predominate over diffusion process. As a result, the amount of analyte captured onto the waveguide is increased, while the time required for doing so is decreased. The flow of fluid also deters non-specific binding of other material in the sample to the waveguide and mixing flow chamber.

However, it should be apparent to one skilled in the art to which the invention pertains that alternative shapes of the mixing flow chamber can also be used to carry out the object of the present invention. The mixing flow chamber-does not need to be rectangular. It can be any shape that causes the fluid to mix. The undulation of the walls, for example, (a) can be periodic or non periodic, (b) can have sharp corners as shown in FIG. 20a or smooth curves (c) can vary only in two coordinates called two-dimensional as shown in FIG. 20a or can vary in three coordinates called three-dimensional, (d) can have undulation on one wall, two walls, three walls, or all sides (e) can have a different undulating pattern on each wall, and any other variation or combination of these features. The shape of the walls should be chosen such that the fluid sample is forced to flow around the waveguide. The shape of the walls should not result in pockets of stagnation.

Mixing flow chamber 240 in FIGS. 20a-c can be made from any material chemically compatible with the analyte and the fluid solution being assayed. In addition, the mixing flow chamber can be either rigid or elastic, and can be a single material or a composite or multilayer structure. The radiation transmissive top surface 220 is preferably very low loss, fabricated from such materials as glass, plastics such as polycarbonate, polystyrene, polyacrrylic, or other clear material. The outside surface of the mixing flow chamber wall 220 can be coated with non-reflective coating to increase the light impinging on the analyte capture surface of the waveguide. The side walls 231 and 232 preferably are made with radiation absorbing material with the property of black color and non reflective, including but not limited to plastics or any other easily molded materials.

In addition, some of the walls of the mixing flow chamber 240 can be coated with reflective material. For example, the surface 236 on wall 230 of FIG. 20c can be coated with reflective material to reflect the excitation light back towards the waveguide to increase the power density of excitation light impinging on the emission detection reagent for the purpose of increase the emission signal.

The waveguides are elongated objects, with a long dimension and shorter cross-sectional dimension. The analyte capture surfaces of the waveguide can be part of the mixing flow chamber wall, but typically they are within the mixing flow chamber but not part of the wall. The waveguides can be oriented along the long axis of the mixing flow chamber or along the short axis of the mixing flow chamber. The flow of the sample can be along the long dimension of the waveguide or perpendicular to the long dimension of the waveguide.

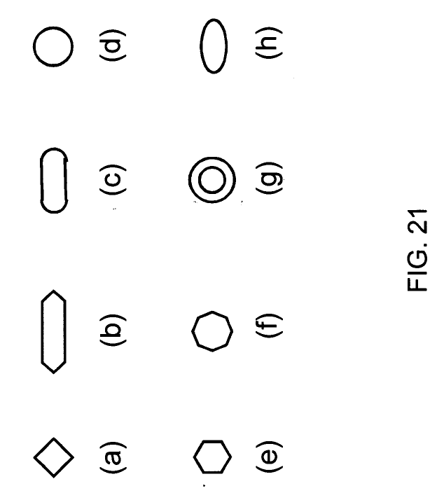

In the embodiment depicted in FIG. 20c, the waveguide member 101 has a rectangular shape. Alternative geometries of waveguide 101 can also be used to carry out the object of the invention. Referring to FIGS. 21a-21h, a non-exhaustive list of several waveguide geometries is presented. As shown in these Figures, a cross-section of waveguide member 101 can have a circular shape, square shape, a ring shape, a polygonal shape (for example, rectangular, trapezoidal, hexagonal or octagonal shape), an annular shape, an oval shape, or any combination or permutation of these and any other useful shape that can guide electromagnetic radiation. In this invention, the waveguide can have any cross-sectional solid or hollow shapes that have low propagation loss in the long direction. In other words, the present invention is not limited to a particular waveguide shape.

The optically clear top of the mixing flow chamber wall 220 and/or the optically clear bottom of the mixing flow chamber wall 230 can also act as waveguides, with the analyte capture surface on the waveguide. In this invention, the waveguide can be made with any material transparent to the excitation light and into which the light emitted by the emission detection reagent can be guided. Typically, waveguide member 101 in one embodiment of the invention can be made of, but not limited to, glass, polymers, optical epoxies, quartz, polypropylene, polyolefin, polystyrene, etc.

The waveguide length can be same as, shorter than or longer than the mixing flow chamber. It can extend outside the mixing flow chamber on one end or on both ends.

As is well known in the art, the ability of a waveguide to confine and direct the propagation of light is dependent on the index of refraction of the waveguide material as well as the index of refraction of material in close proximity. The higher the index of refraction of the waveguide material, compared to its surroundings, the better the waveguide can confine the light for identical geometries. Therefore, it is preferable that the refractive index of waveguide member 101 have a value greater than the refractive index of the medium surrounding the waveguide inside the mixing flow chamber.

The waveguide surface can be multi-layered. In fiber optics, for example, a thin layer of cladding, a material with an index of refraction less than that of the core material, is used to better confine the emitted radiation within the fiber. This same principle can be applied to the waveguide. All or parts of the waveguide can consist of a core surrounded by a cladding. In the embodiment described in FIGS. 20a-c, as an example, all of the parts of the waveguide member 101 except some portions in the interior of the mixing flow chamber 240 are provided with, but not required to have, a cladding. The cladding is generally made of glass or plastic. The cladding performs the following functions: reduces loss of light from the core into the surrounding, reduces scattering loss at the surface of the core, protects the fiber from physical damage and absorbing surface contaminants, and adds mechanical strength.

Part of the cladding can be covered with a coating, or “jacket”. The coating is more desirable outside the mixing flow chamber. The coating serves to physically protect the waveguide member from the outside materials and to prevent any parasitic or environmental radiation from entering into the waveguide.

Some of the waveguide can have a portion outside the mixing flow chamber, part 104 or none at all extending outside the mixing flow chamber.

Parts of the waveguide can be covered with reflective material. In the embodiment described in FIG. 20b, a reflective member 215 can be provided at the first end of the waveguide 102, but this is not required. This member reflects light towards the direction of the detector end of the waveguide 103. Preferably, reflective member 215 is composed of a coating of material that specifically reflects the radiation emitted by the emission detector reagent. It can also be desired that this coating of material absorbs the excitation light in order to limit background radiations reaching the detector. Reflective member 215, which is secured at the first end 102, can also be affixed at first end 233 of mixing flow chamber 240, as is represented in the embodiment of FIG. 20b.

It is not desirable to have the left side 105 and right side 106 of waveguide 101 as shown in FIG. 20c to be analyte capture surface because there would not be adequate amount of excitation light impinging on side 105 and 106 to impinge on the emission detection reagents. Furthermore, covering the sides 105 and 106 with reflective material, cladding material or other materials different from the waveguide are methods to accomplish this goal and improve transmission of the emission signal to the detector.

The waveguide end 102 can be used to manipulate the reflection of total light power and also be used to manipulate the reflection of light as a function of wavelength. The use of reflective material is one method of obtaining nearly total reflection for a range of wavelengths emission detection reagents. Semi-circular shaped ends can also provide good reflection of light. To obtain frequency selective reflections, multi-layer coatings or gratings can be used, for example, to obtain high reflection of light produced by the labels and low reflection of excitation light. For example, an optical grating is fabricated on the inside of an optical fiber. The grating end of the optical fiber is placed just before the detector. The signal collected by the fiber constitute of both the emission and excitation light. The grating will provide high transmission of the light produced by the labels and low transmission of excitation light to the detector. Thus, the signal to noise ratio of some detectors can be improved.

The number of waveguides inside a mixing flow chamber can be more than one. The arrangement of the waveguides inside the mixing flow chamber can vary.

The inlet 260 and outlet 261 are located on the mixing flow chamber surface 230 opposite that of the radiation transmissive surface 220 in FIG. 20b, but it could be on any part of the boundaries of the mixing flow chamber 240, including sides 231, 232, 233 and 234, or top 220. There can be more than one inlet and more than one outlet. The inlets and the outlets do not have to be on the same wall. The inlets and outlet can have different dimensions and any construction.

FIG. 20a shows one detector system at the end of waveguide end 103. Another detector system can also be implemented at the waveguide end 102, instead of a reflective mirror.

The mixing flow cartridge can contain not only the mixing flow chamber and the waveguide, but can also contain a sample chamber, and chambers that store reagents needed for the assay and waste products from the assay and other preparatory processes.

Signal detection is performed with a detector member 270 provided at one or both extremities of waveguide member 103. The detector member 270 is part of the sensor instrument. Generally, detection can be performed without the presence of fluid inside the chamber. Yet, it should be kept in mind that detection in the present invention can also be done with a fluid continually flowing through the mixing flow chamber. For instance, detection can be done with a reagent or a rinse present inside the mixing flow chamber. Detector member 270 is constructed and arranged to receive a signal exiting second end 103 and to provide quantitative and qualitative information about the assayed sample.

As mentioned previously, the sensing system 200 can be embedded in a sensor instrument. This instrument should be designed to facilitate the mixing flow cartridge installation, radiation illumination and detection. The instrument includes all of the elements necessary to perform detection and analysis in any type of environment. The instrument can also include other functions.

This instrument can be used in the following way for one type of sandwich immunoassay, an example of which is described in this paragraph. First, the sample containing the analyte is introduced into the mixing flow cartridge. A system of filters interposed before the inlet can be used to prevent large particles from entering and clogging the mixing flow chamber. In this mode of operation, the analyte capture surface specific to the analytes has been coated in advance on the waveguide member. The sample containing the analyte is flowed inside the mixing flow chamber between the inlet and the outlet. Analyte that is specific to the capture antibody binds to the waveguide member via the capture antibody, while other matter present in the solution is flushed out of the mixing flow chamber. A rinse can be provided in order to eliminate unbound analyte and any matter that has been partially or non-specifically bound to the waveguide or other surfaces in the mixing flow chamber. For sandwich assay, the emission detector reagent, comprising the luminescent labeled detector antibody elements, can next be introduced and to bind to the analyte of the analyte/capture antibody complexes, thereby completing the sandwich assay. A further rinse step can be performed to eliminate unbound emission detection reagent. Then, the waveguide is illuminated by a light source. The illumination can take place while rinsing solution is still inside the mixing flow chamber or while the mixing flow chamber is empty. Finally, the signal produced by the emission detection is captured by the waveguide member and guided to the radiation detection member.

This instrument can be used in another way for a second types of sandwich immunoassay, an example of which is described in this paragraph. The waveguide is coated with avidin. First, one or more filters are used to extract large debris from the sample containing the analyte. This is followed by mixed the sample with emission detection reagent and the analyte recognition coating. The analyte recognition coating can be biotinylated antibody. The analyte of interest will be coated with both the analyte recognition coating and the emission detection reagent. The unbound analyte recognition coating and the emission detection reagent can be filtered out and the analyte along with other particulars will be washed and re-suspended in buffer. The re-suspended solution is flowed inside the mixing flow chamber between the inlet and the outlet. Analyte that is specific to the analyte recognition coating binds to the analyte capture surface on the waveguide member via the avidin-biotin binding, while other matter present in the solution is flushed out of the mixing flow chamber. A rinse can be provided in order to eliminate unbound analyte and any matter that has been partially or non-specifically bound to the waveguide or other surfaces in the mixing flow chamber. The waveguide is illuminated and the signal produced by the labels on the surface of the waveguide is captured by the waveguide member and guided to the radiation detection member.

This instrument can also be used in other types of sandwich assays.

There are many other types of assays. This sensor is not limited to the sandwich immunoassay.

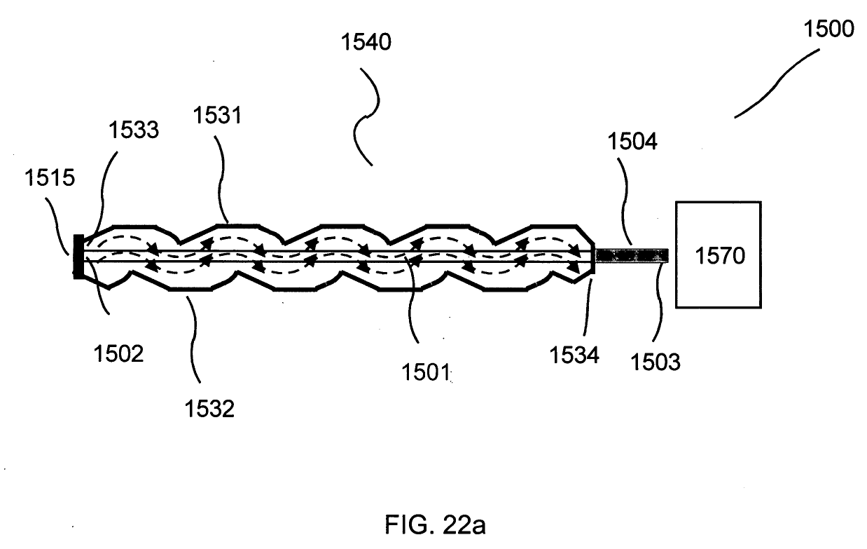

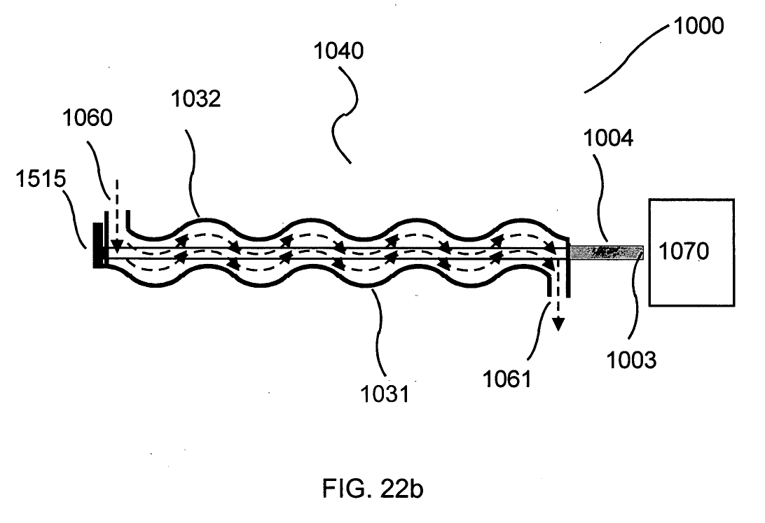

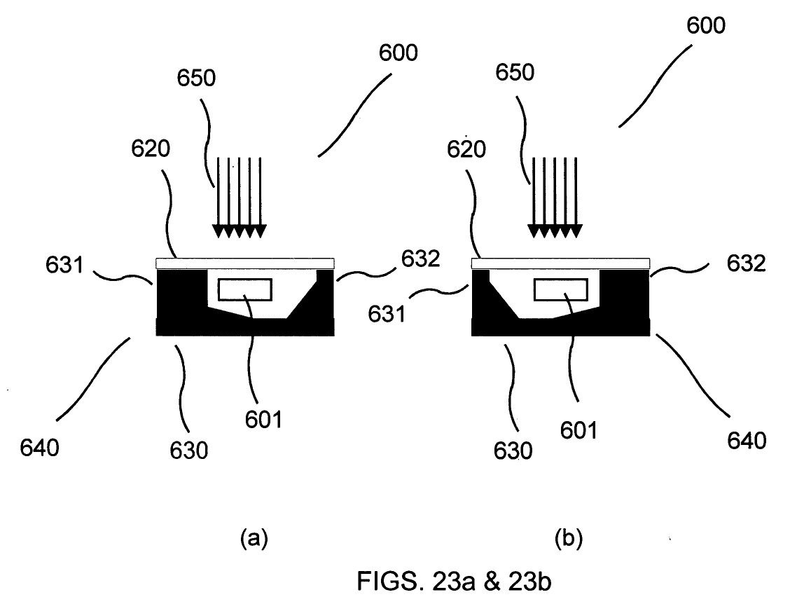

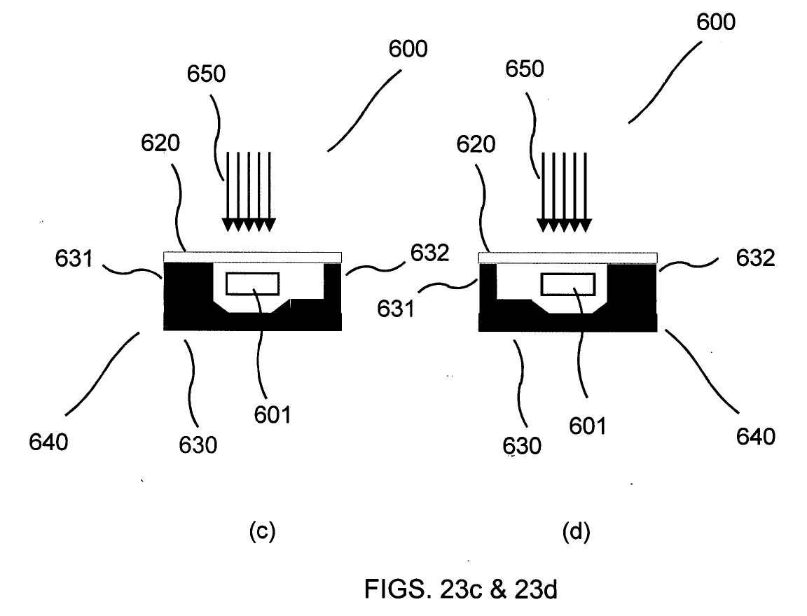

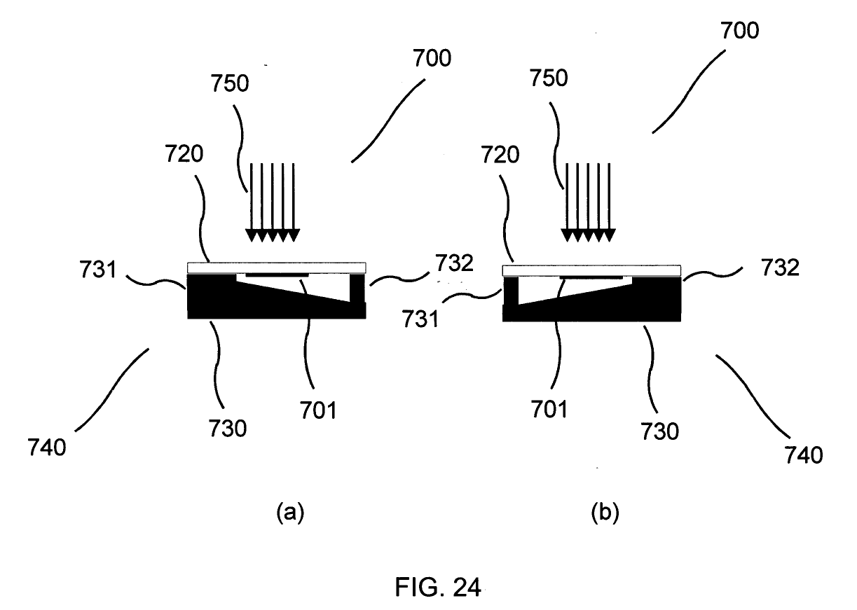

As mentioned in the foregoing discussion, the surface of the mixing flow chamber can have a shape that differs from the one represented in FIGS. 20a-c. Referring to FIGS. 22a and 22b, two alternative examples of side view shapes are provided. Referring to FIGS. 23a, 23b, 24a and 24b, alternative examples of end view surface shapes are provided. The embodiment of the possible shapes of the mixing flow chamber is not limited to these examples.

As shown in FIGS. 22a and 22b, the side views of the mixing flow chambers 1540 can have different undulating forms.

Three-dimensional mixing flow surface can also be used in another embodiment of the invention. One such embodiment is represented in FIGS. 23a and b at two axial locations, and another such embodiment is represented in FIGS. 23c and d at two axial locations representing cross-sectional end-views of a mixing flow-through sensor according to different embodiments of the invention. Similar to the embodiment depicted in FIG. 20c, the sensing system 600 comprises waveguide member 601 that is located inside the elongated body of mixing flow chamber 640. Elongated body 640 includes top transmissive portion 620, which is transparent to the beam of radiation 650 impinging on waveguide member 601. Elongated body 640 further includes side member 631 and 632 and a bottom member 630. Like the embodiment shown in FIG. 20c, boundary members 630, 631 and 632 can be made capable of absorbing the excitation light and therefore reduce the scattering of light towards the detector member. FIGS. 23a-b and 23c-d illustrate the mixing flow chamber 640 constructed such that (a) at certain positions the left wall 631 is closer to the waveguide and (b) at other positions the right wall 632 is closer to the waveguide, respectively. FIGS. 23a-b and 23c-d not only show that the side walls are undulated, but that the bottom wall 630 also undulates in the long direction.

FIGS. 24a and b present cross-sectional end-views at two axial locations of another embodiment of the mixing flow-through sensor where there is no waveguide in the interior of mixing flow chamber 740. Similar to the embodiment depicted in FIG. 20c, the sensing system 700 consists of an elongated body 740 and transmissive top member 720. A portion of the transmissive top member 720 is coated with the analyte capture surface 701 and the transmissive top member 720 also serves as the waveguide. Elongated body 740 further includes side member 731 and 732 and a bottom member 730. Like the embodiment shown in FIG. 20c, boundary members 730, 731 and 732 are capable of absorbing the radiation emitted by the light source and can therefore reduce the scattering of light towards the detector member. FIGS. 24a and 24b illustrate the mixing flow chamber 740 at the positions (a) where the members 730 and 731 are closer to the waveguide and (b) where the members 730 and 732 are closer to the waveguide, respectively.

Although several illustrations of mixing flow surfaces have been provided in the foregoing discussion, it should be understood that other mixing flow members or mixing flow surfaces can be suitable to carry out the object of the invention.

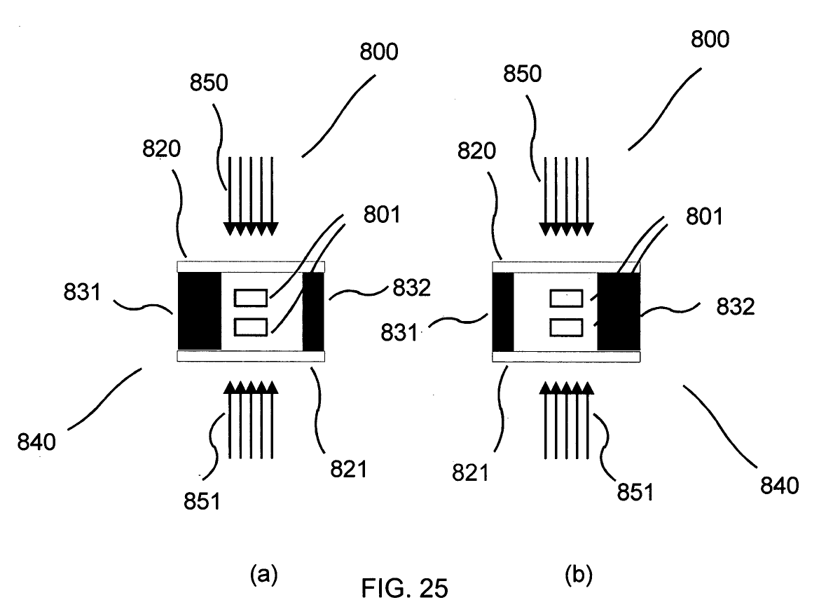

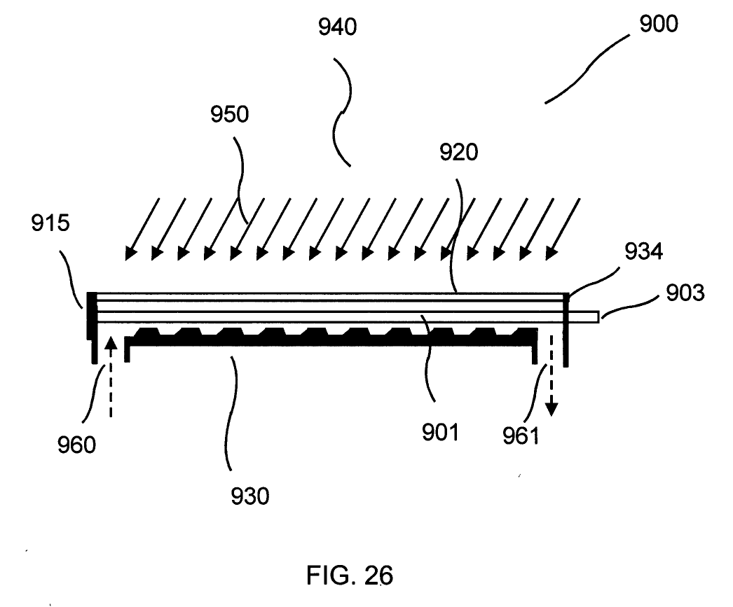

In order to increase the signal received by the detector member, the mixing flow chamber can optionally contain more than one waveguide. Such embodiment is represented in FIGS. 25a and bpresenting two cross-sectional end-views of a mixing flow-through sensor at two different axial locations. The sensing system 800 consists of two waveguide members 801 that are located inside elongated body of mixing flow chamber 840. Elongated body 840 includes top transmissive member 820 and bottom transmissive member 821, which are transparent to the excitation light 850 and 851 impinging on waveguide members 801. Like the embodiment shown in FIG. 20c, boundary walls 831 and 832 can be made capable of absorbing the radiation emitted by the excitation light. FIGS. 25a and 25b illustrating the mixing flow chamber 840 at the positions that (a) the left wall 831 is closer to the waveguide and (b) the right wall 831 is closer to the waveguide, respectively.

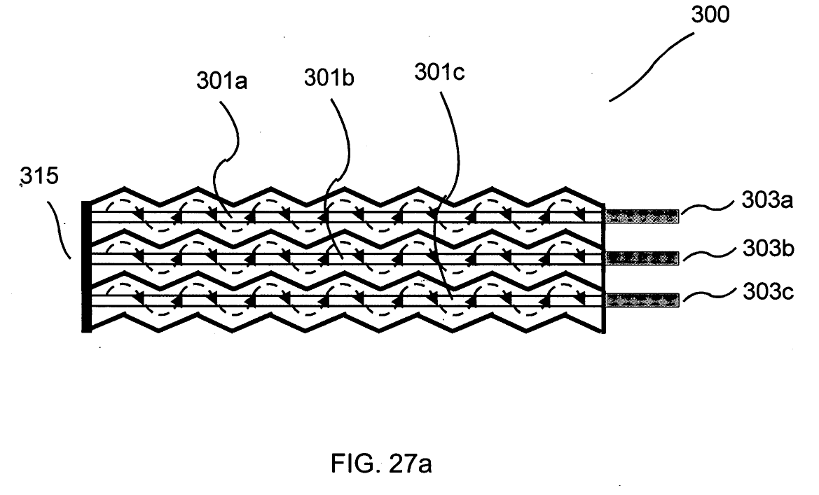

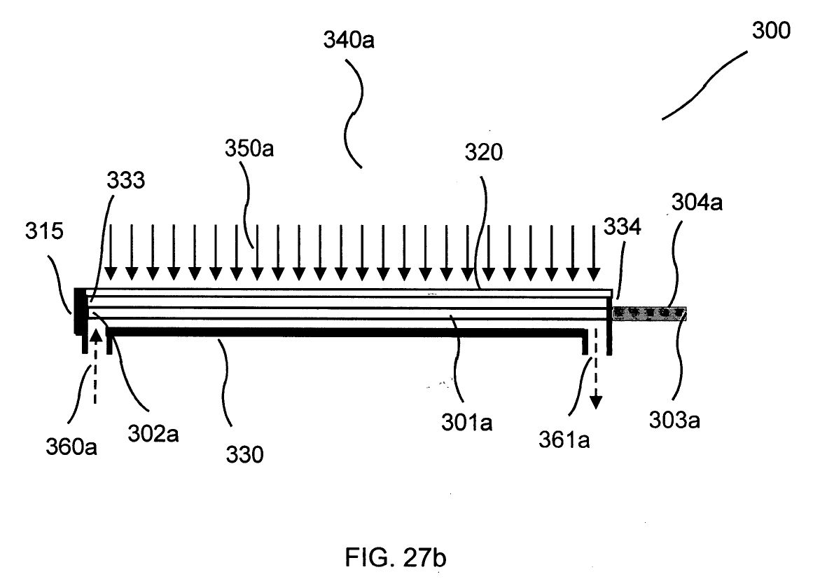

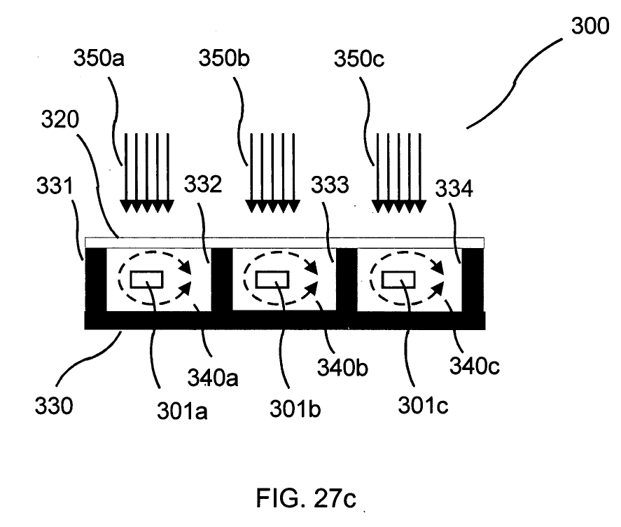

FIG. 27 presents side view of a mixing flow-through sensor according to another embodiment of the invention at two different axial locations. The sensing system 900 comprises waveguide members 901 that are disposed inside elongated body of mixing flow chamber 940. The end of the waveguide 903 is unobscured by the waveguide wall 934 to let the emission light out to the detector, but not extended outside the wall 934. Elongated body 940 includes top transmissive member 920 and bottom light absorbing member 930 and an inlet 960 and outlet 961. The bottom wall member 930 is undulating. The excitation light 950 is collimated but not perpendicular to the long direction of the waveguide.

FIGS. 27a-c depicts a top view, side view and an end view of the multi-analyte sensor according to one embodiment of the invention, respectively. Multi-analyte sensor 300 comprises a plurality of mixing flow chambers, each of them housing a waveguide member capable of conveying the emitted light to a detector member (not shown in FIG. 27a). The multi-analyte sensor 300 is constructed and arranged to identify and quantify different analytes at the same time or at different times. It also allows for an optimal construction of the sandwich assays on each of the waveguides due to the mixing flow chamber.

Multi-analyte sensor 300 comprises a plurality of mixing flow chambers 340a, 340b and 340c grouply secured. Like the embodiment depicted in FIG. 20a, each of the mixing flow chambers 340a, 340b and 340c respectively comprise a waveguide member 301a, 301b and 301c on which is the analyte capture surface so as to substantially expose the entire analyte capture surface of the waveguide to the sample in the interior of the mixing flow chamber. The first end of each waveguide can be coated by a reflective or multi-layered material or shaped to improve the reflection of emitted radiation and reduce the reflection of the emitted radiation. The second end of each waveguide can be unobscured to allow the transmission of the emitted radiation out of the waveguide to the detector system (not shown).

FIG. 27b shows the side view of the mixing flow chamber 340, which is further comprised of radiation transmissive surface 320 allowing the excitation light to propagate to the analyte capture surface on the surface of the waveguide. The lower border 330 can be clear, black or any other color or coated with reflective or absorbent material. The mixing flow chamber includes inlets 360a-c and outlets 361a-c to allow a fluid solution to flow inside the mixing flow chamber between inlet 360a-c and outlets 361a-c, respectively. The excitation light 350 impinges directly or indirectly on the waveguide surface.

FIG. 27c shows the end view of the sensing system 300, including radiation transparent top boundary 320, the side members 331, 332, 333 and 334, the bottom wall 330, the waveguides 301a, 301b and 301c, and incident radiation 350.

Mixing flow surfaces are constructed and arranged to maximize the interaction between the constitutive elements of the fluid solution and the waveguide member. Specifically, mixing flow surface is constructed and arranged so that the fluid flowing inside each mixing flow chamber is in a non-laminar regime.

While the number of mixing flow chambers is limited to three in the embodiment of FIGS. 27a-c, it should be apparent to one skilled in the art to which the invention pertains that multi-analyte sensor can comprise a larger number of mixing flow chambers. Generally, the number of mixing flow chambers depends on the application needs and can be determined by the size of the instrument.

All the variations of inventions described earlier for FIGS. 20a-c are also applicable to this multi-mixing flow chambers sensor embodiment, FIGS. 27a-c.

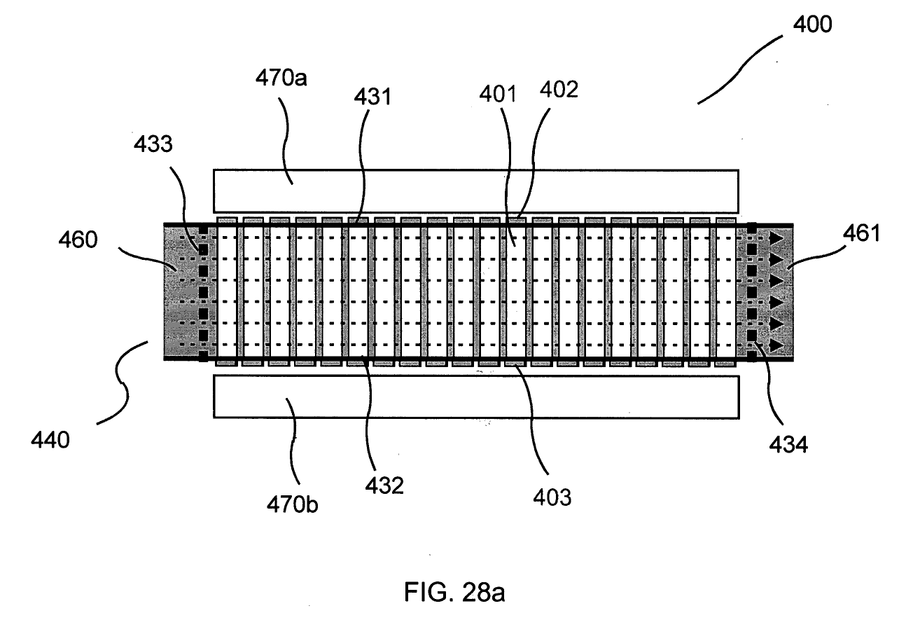

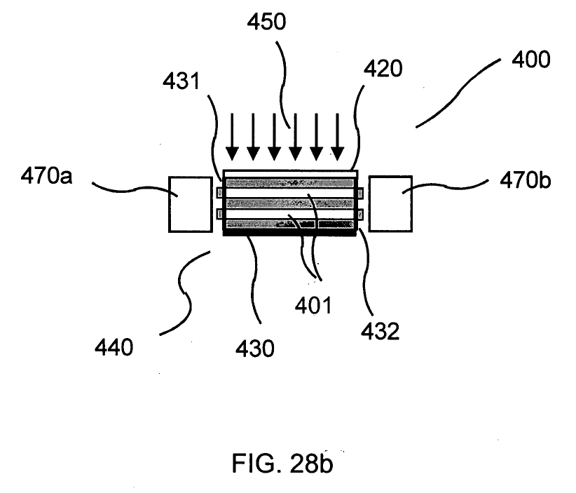

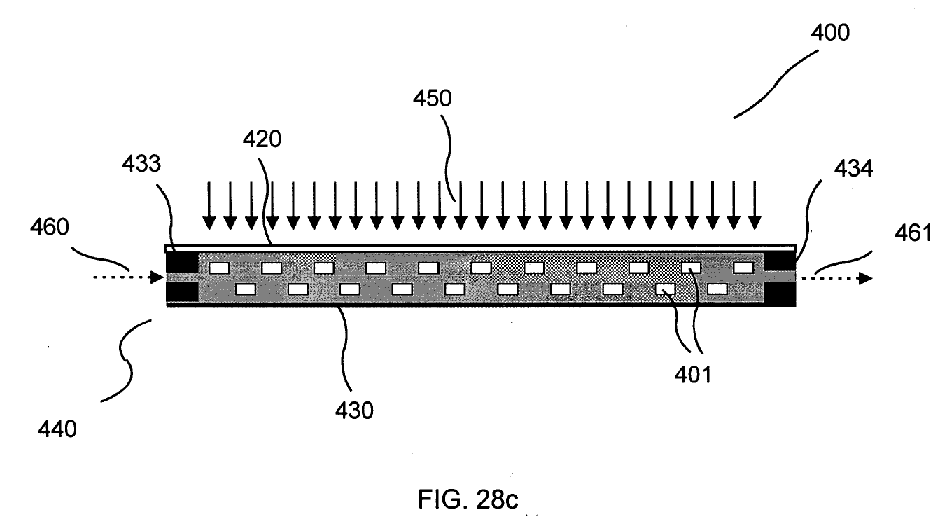

FIGS. 28a-c show the top view, side view and end view, respectively, of a mixing flow-through sensor providing fast flow rate and rapid capture of the analyte according to another embodiment of the invention. Sensing system 400 is comprised of a mixing flow chamber 440, with a large number of waveguide members 401 coated with analyte capture surface, and the detector system members 470a and 470b.

In the embodiment shown in FIG. 28a, waveguide members 401 consist of a number of elongated members, adapted to propagate along their lengths the collected emission signal. Sensor 400 comprises a plurality of waveguide members 401 in the mixing flow chamber 440, so as to expose substantially all of the waveguide surface to the sample, leaving first end 402 and second end 403 of the waveguide unobscured. More particularly, mixing flow chamber 440 comprised of an elongated side bodies 431 and 432 that extends outward from waveguide member 401 and is constructed and arranged to contain a portion of the waveguides. The waveguides 401 are positioned approximately perpendicular to the flow of the sample. The side members 431 and 432 are secured to waveguide members 401. The inlet 460 and outlet 461 allow a fluid sample to flow inside the mixing flow chamber between inlet 460 and outlet 461 and they are formed by holes through mixing flow chamber walls 433 and 434, respectively. Two sets of detector members 470a and 470b can be used to detect light exiting from the waveguide ends 402 and 403.

As the fluid is flows through in the mixing flow chamber over the waveguides, the analytes in the fluid has improved chance of being captured if the number of waveguides is increased. The waveguides can capture one or more varieties of analytes.

FIG. 28b shows the side view of the mixing flow chamber 440 further comprises of radiation transmissive surface 420 allowing the excitation light to propagate to the analyte capture surface on the surface of the waveguide, the side walls 431 and 432, and the lower border 430, which can be clear, be black or be coated with reflective material. The excitation light 450 impinges directly or indirectly on the waveguide surfaces. The emission signal exits from the waveguide ends to enter the detector members 470a and 470b.

FIG. 28c shows the end view of the sensing system 400, including radiation transmissive top boundary 420, the bottom wall 430, side walls 433 and 434, inlet 460, outlet 461, the waveguides 401 and incident radiation 450.

The number of waveguides, their position, and length can vary. Only one of the detector systems can not be necessary. The inlet 460 and outlet 461 can be located on the bottom wall 430.

All the variations of inventions described earlier for FIGS. 20a-c are also applicable to this multi-mixing flow chambers sensor embodiment, FIGS. 28a-c.

The mixing of the fluid is caused by waveguides because they are positioned in the path of the fluid flow. The waveguides are constructed and arranged to maximize the interaction between the constitutive elements of the fluid solution and the waveguide member. Specifically, mixing flow surface is constructed and arranged so that the fluid flowing inside each mixing flow chamber is in a non-laminar regime.

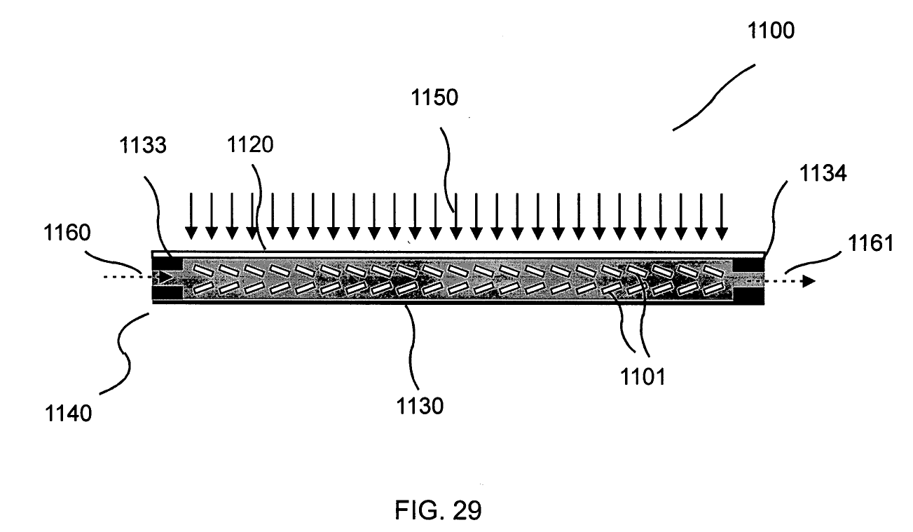

FIG. 29 shows another embodiment of the end view of the sensing system 400. The waveguides are positioned to allow a different flow. FIG. 29 shows the end view of the sensing system 1100, including radiation transmissive top boundary 1120, the bottom wall 1130, side walls 1133 and 1134, inlet 1160, outlet 1161, the waveguides 1101 and incident radiation 1150. When this embodiment is coupled with pulsed, reversible flow direction, the mixing flow chamber 1140 can also provide efficient fluid sampling by the waveguide analyte capture surface.

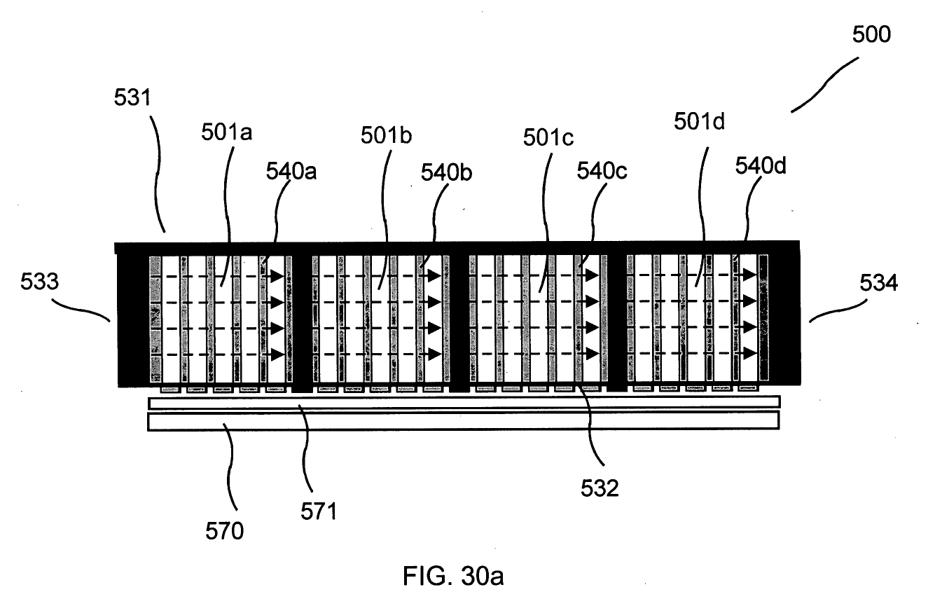

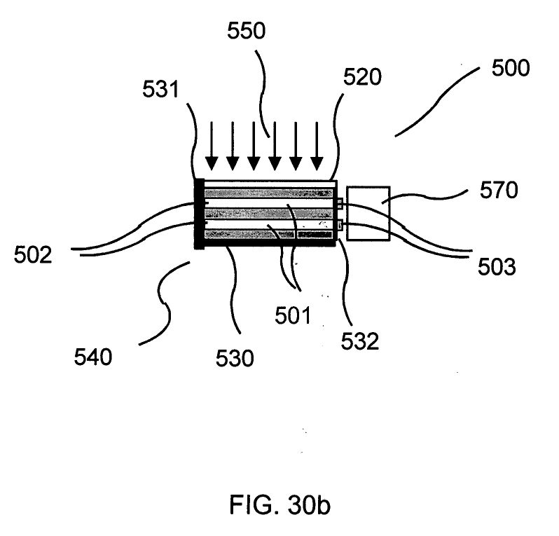

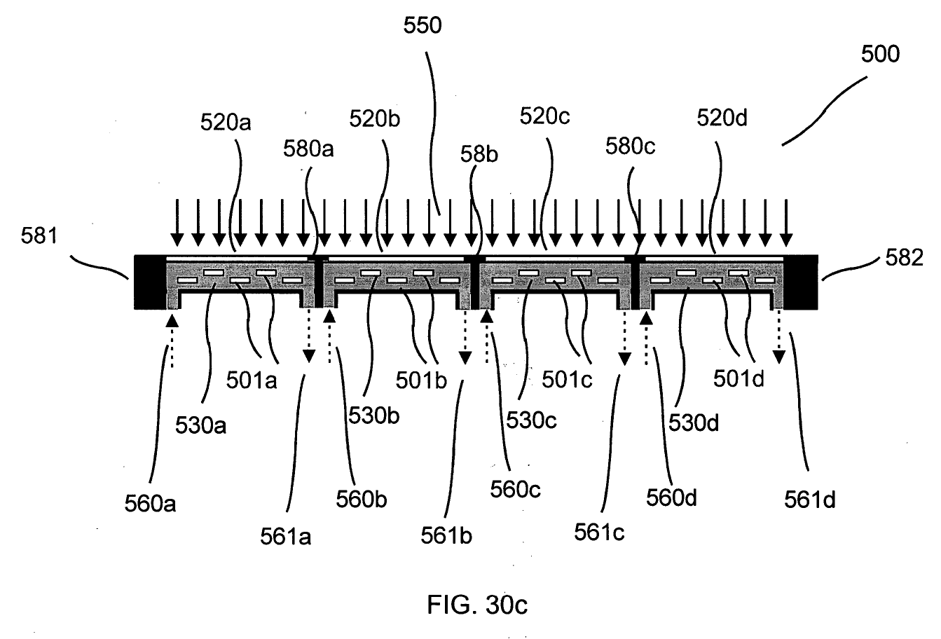

FIGS. 30a-c show the top view, side view and end view of a multi-analyte mixing flow-through sensor according to another embodiment of the invention, respectively. This embodiment is applicable for testing large volumes of samples over large number of waveguides to enable more rapid analyte capture. Sensing system 500 comprises mixing flow chambers 540a, 540b, 540c and 540d, with a large number of waveguide members 501a, 501b, 501c and 501d, coated with analyte capture surface, and the detector systems member 570.

In the embodiment shown in FIG. 30a, the sensing system 500 consists of a number of mixing flow chambers 540a, 540b, 540c and 540d. The waveguide members 501a, 501b, 501c and 501d are situated in the mixing flow chambers 540a, 540b, 540c and 540d, so as to expose substantially all of the waveguide surface to the sample, leaving first end 502 and second end 503 of the waveguide unobscured. The waveguides 501a, 501b, 501c and 510d are positioned approximately perpendicular to the flow of the sample. The waveguide members 501a, 501b, 501c and 501d are secured to the side members 531 and 532. The emission signal exits from the waveguides and enters the detector member 570.

FIG. 30b shows the side view of the mixing flow chamber 540 further comprises of radiation transmissive surface 520 allowing the excitation light to propagate to the analyte capture surface on the surface of the waveguide, the side walls 531 and 532, and the lower border 530, which can be clear, black or any other color, or coated with reflective or absorbent material. The excitation light 550 directly incident on the waveguide surfaces. The waveguide end 502 can be coated with a reflective material. The emission signal exits from the waveguide end 503 to enter the detector system 570.

FIG. 30c shows the end view of the sensing system 500, including radiation transmissive top boundary 520, the bottom wall 530, side walls 531 and 532, the waveguides 501 and incident radiation 550. The fluid enters each mixing flow chamber 540a, 540b, 540c and 540d through inlets 560a, 560b, 560c and 560d and exit through outlets 561a, 561b, 561c, and 561d, respectively.

The mixing of the fluid is caused by waveguides because they are positioned in the pass of the fluid flow. The waveguides are constructed and arranged to maximize the interaction between the constitutive elements of the fluid solution and the waveguide member. Specifically, mixing flow surface is constructed and arranged so that the fluid flowing inside each mixing flow chamber is in a non-laminar regime.

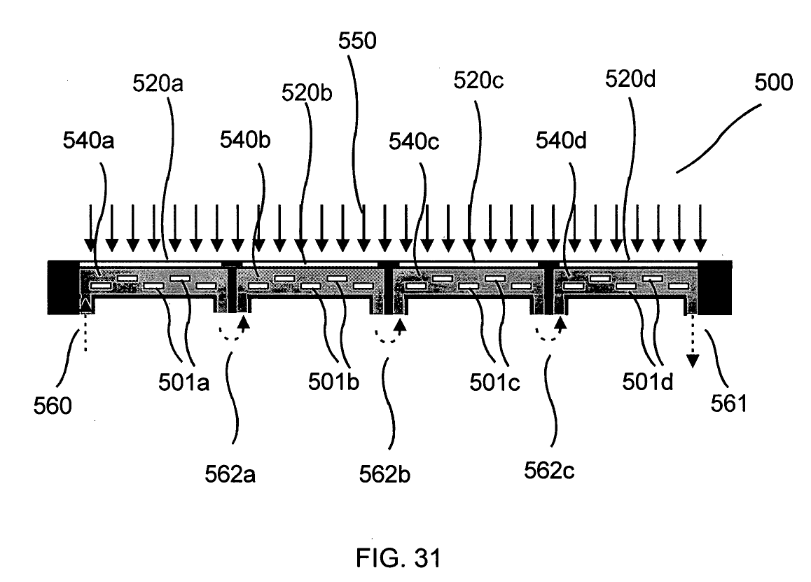

FIG. 31 shows the flow of the sample over all waveguides is achieved by sending the sample from outlets from one mixing flow chamber to the inlet of the next mixing flow chamber during the analyte capture phase. During the rest of the procedures, the solutions from one mixing flow chamber preferably do not go to the next mixing flow chamber.

The number of waveguides, their position and lengths can vary. Detector systems can be used on either or both ends of the waveguides. The inlet 560 and outlet 561 can be located on the bottom wall 530.

All the variations of inventions described earlier for FIGS. 20a-c, FIGS. 30a-c are also applicable to this multi-mixing flow chamber sensor embodiment,

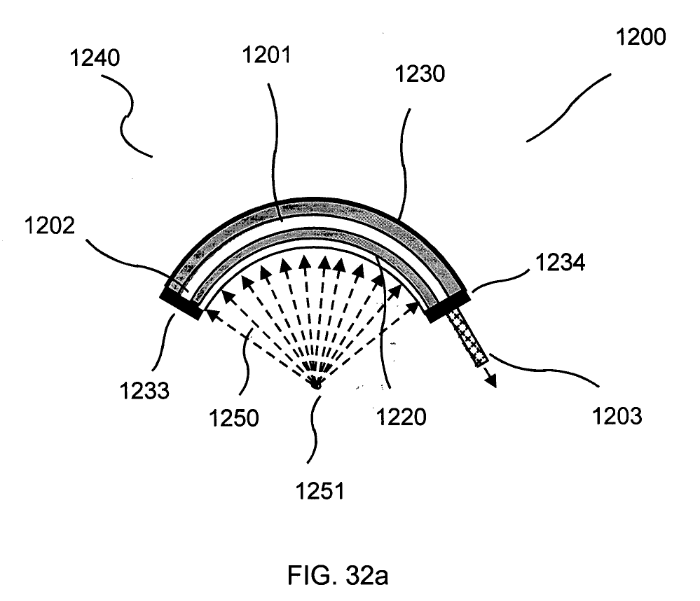

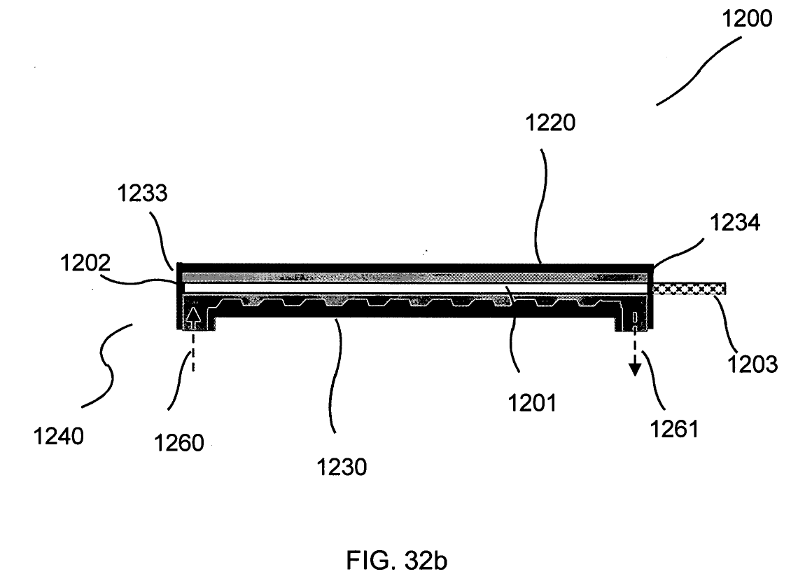

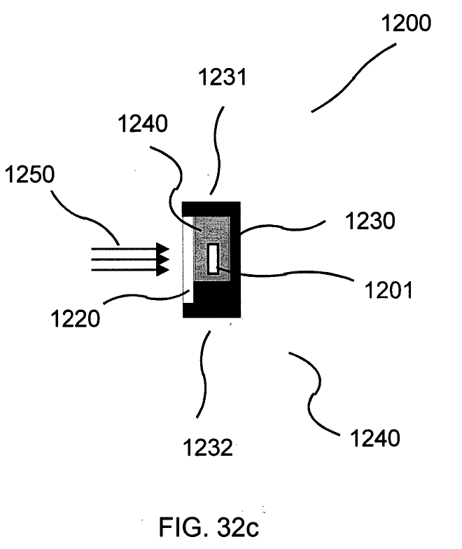

The sensor can also be achieved with an embodiment utilizing a diverging light source as shown in the cross sectional top view, side view and end view represented in FIG. 32a, 32b and 32c, respectively. It provides a perpendicular irradiation without using an optical system, thereby reducing the size and optics associated with the system.

In this embodiment shown in FIG. 32a, the mixing flow chamber 1240 is bent in the form of a section of a circle. The mixing flow chamber 1240 is comprised of elongated side bodies 1220 and 1230, and end bodies 1233 and 1234. The waveguide 1201 is secured to the end members 1233 and 1234.

Light from a point source 1251 diverges in a fan beam 1250 and it perpendicularly impinges onto radiation transmissive surface 1220 of mixing flow chamber 1240. The waveguide is a curved elongated member 1201, adapted to propagate along its length the collected emission signal. The waveguide member 1201 passes through mixing flow chamber 1240, so as to expose substantially all of the waveguide surface to the sample, leaving first end 1202 and second end 1203 of the waveguide unobscured. A reflective surface can be placed on the first end of waveguide 1202. The emission signal is transmitted out of the end 1203 into detector member (not shown).

FIG. 32b shows the side view of the mixing flow chamber 1240 seen through the center of the waveguide. The mixing flow chamber 1240 is further comprised of radiation transmissive surface 1220 allowing the excitation light to propagate to the analyte capture surface on the surface of the waveguide, the end walls 1233 and 1234, and inlet 1260 and outlet 1261. The undulating border 1230 can be made of light absorbing material. The undulating boarder 1230 provides the mixing as the sample flows from inlet 1260 to outlet 1261.

FIG. 32c shows the cross sectional end view of the sensing system 1200, including radiation transmissive boundary 1220, the undulating boundary 1230, side walls 1231 and 1232, the waveguides 1201 and incident radiation 1250.

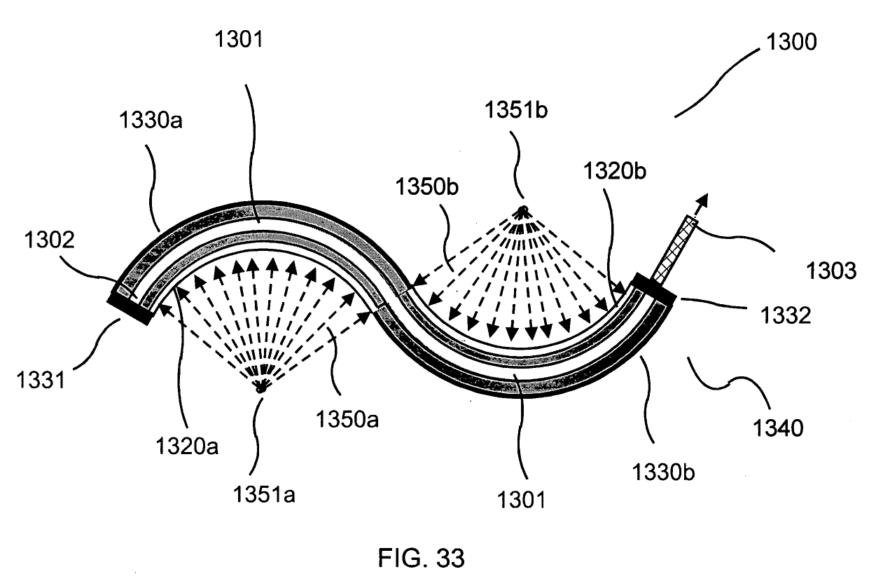

An alternative embodiment that provides multi-analyte sensing and fan light beam is provided in FIG. 33, consisting of two consecutive embodiment shown in FIG. 32a. The sensing system 1300 is comprised of mixing flow chamber 1340 doubly bent such that projection beams 1350a and 1350b perpendicularly impinge onto radiation transmissive surfaces 1320a and 1320b. As can be seen in this embodiment, the irradiation of waveguide 1301 is provided by two diverging light sources 1351a and 1351b, each being disposed towards a circular section of the mixing flow chamber. In this embodiment, each section is illuminated with a cone beam light source and can be used to detect the same analyte or a different analyte. While only two bent sections are provided in FIG. 33, alternative embodiments containing more sections can also be used to carry the object of the invention.

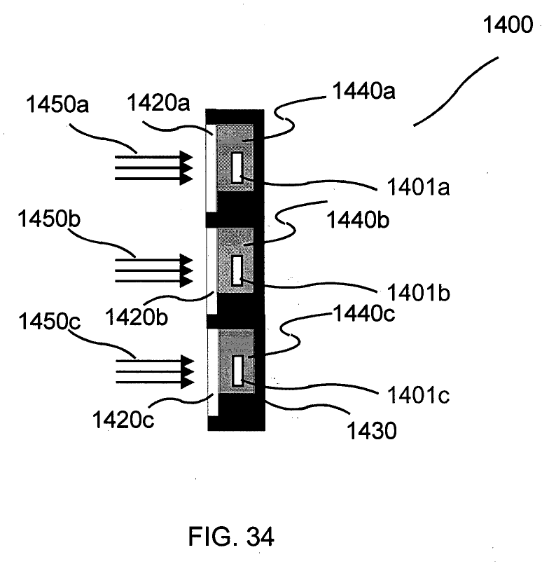

Another alternative embodiment that also provides multi-analyte recognition and fan light beam is provided in the cross sectional end view in FIG. 34. This is applicable to top views shown in FIG. 32a and FIG. 33. The fluid inlet and outlet are to be placed in the wall 1230 in FIG. 32a and wall 1330 in FIG. 33. While the number of mixing flow chambers is limited to three in the embodiment of FIG. 34, it should be apparent to one skilled in the art to which the invention pertains that multi-analyte sensor can comprise a different number of mixing flow chambers. Generally, the number of mixing flow chambers depends on the application needs and can be determined by the size of the instrument.

All the variations of inventions described earlier for FIGS. 20a-c are also applicable to this multi-mixing flow chambers sensor embodiment, FIGS. 32a-c, and 34.

Another alternative embodiment to mix the fluid in the flow chamber is to actuate movable objects in the chamber. For example, the moving objects can be small air bubbles, compressible beads, small magnetic beads or rods. Other means known in the art that facilitate mixing of fluid or fluid-like substances similarly can be used to configure a flow chamber given the teachings and guidance provided herein. The actuation of the objects can be achieved electronically, mechanically, electromechanically, thermally, electromagnetically, magnetically, by vibration or other energy sources. The flow of the sample can be along the length of the waveguide or perpendicular to the length of the waveguide. Two examples among a wide variety of possibilities are given below.

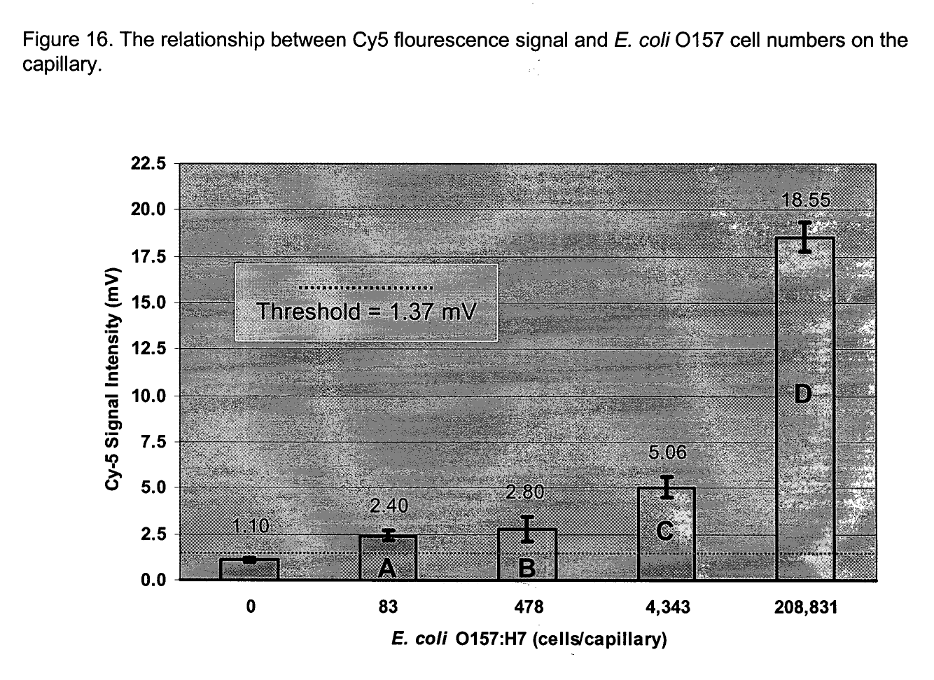

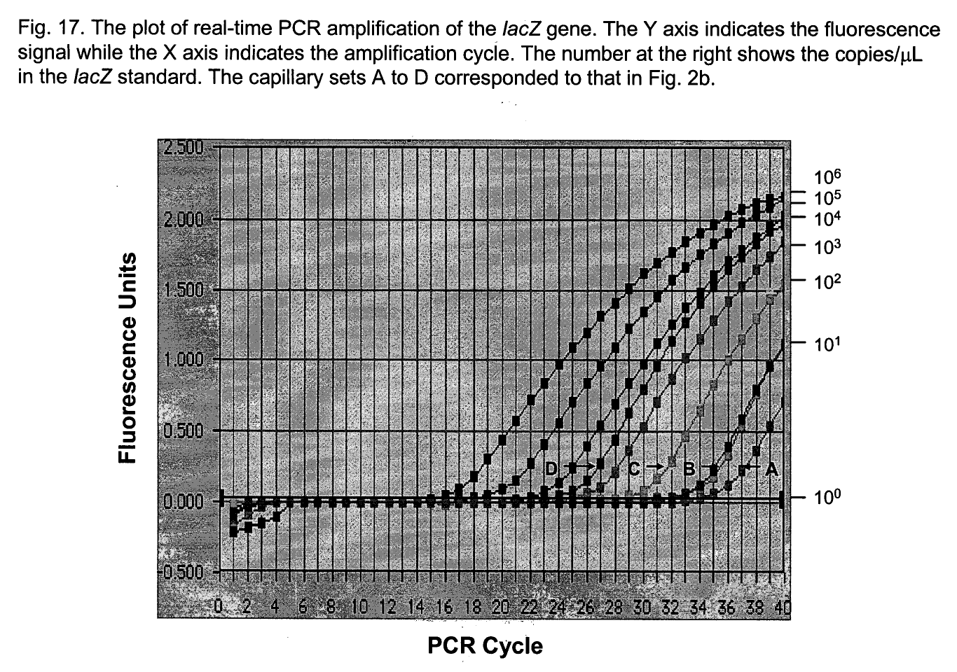

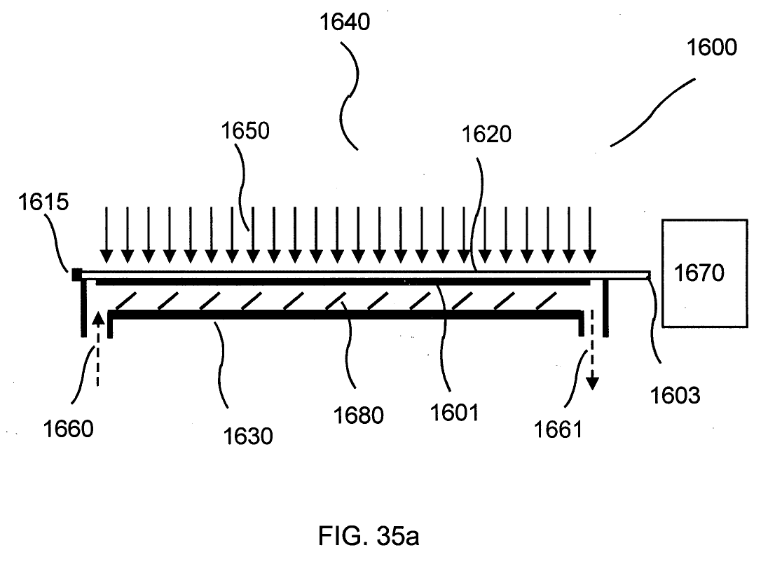

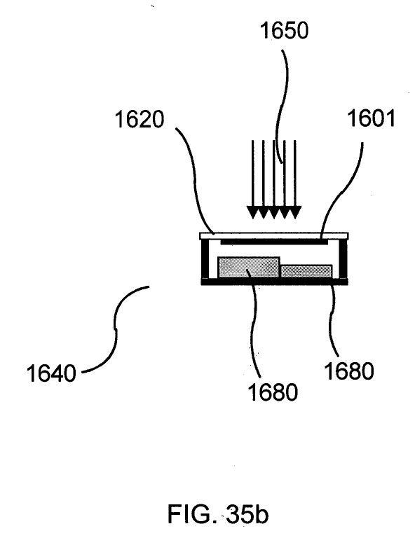

FIGS. 35a and 16b are cross sectional side view and end view of an mixing flow sensor where the flow is along the length of the waveguide and the mixing is achieved by actuation of movable objects 1680 below the waveguide. In this drawing, the waveguide is also the top boundary. The waveguide can also be in the interior of the flow chamber.

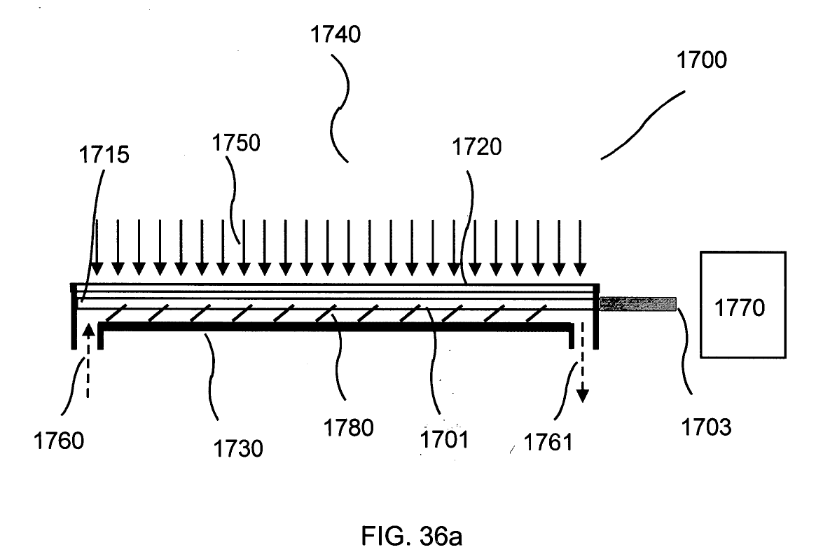

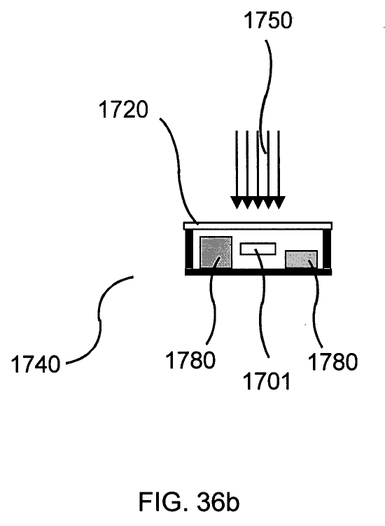

FIGS. 36a and 36b are cross sectional side view and end view of another embodiment where the mixing is achieved by actuation of movable objects 1780 at the sides of the waveguide. The motion of the moving parts on one side can be the same as the moving parts on the other side, but can also be different. The shape of each piece of the moving part can be the same or different. The shape of the moving part can vary and the speed of the motion can also vary temporally.

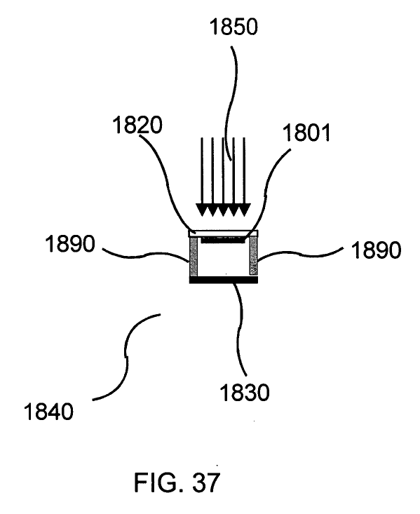

Another alternative embodiment to mix the flow in the flow chamber is to apply an electrical field across the flow chamber in the cross sectional plane. FIG. 37 shows the end view where the application of the electric field is in the vertical direction such that the electric potential on the clear surface 1820 is different from bottom surface 1830 and the side walls 1890 are insulating. Appropriate voltages will be chosen for the analyte to be detected. The flow chamber is not limited to the rectangular shape and the location of the electrodes can vary. The amplitude of the electric field can be uniform or vary in the axial length. The vector of the electric field can also vary in direction along the axial length.

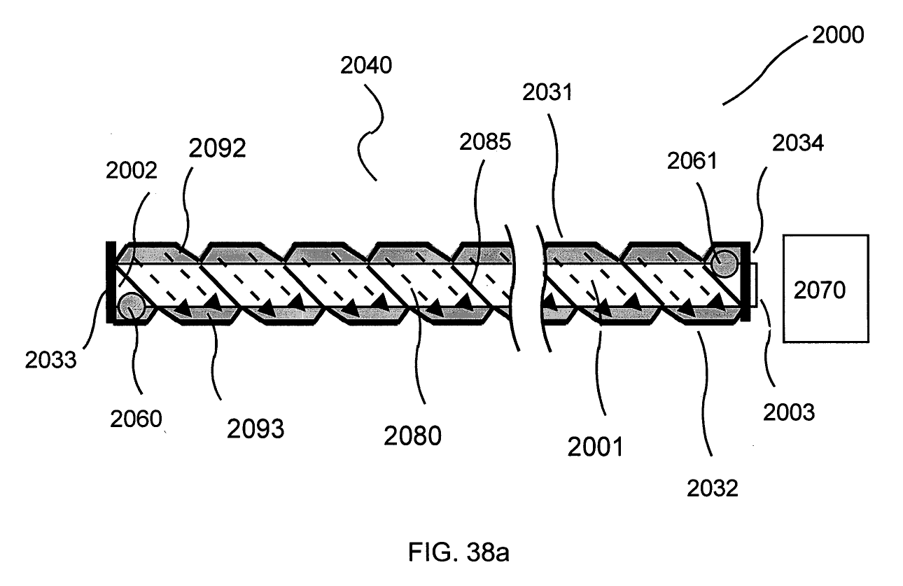

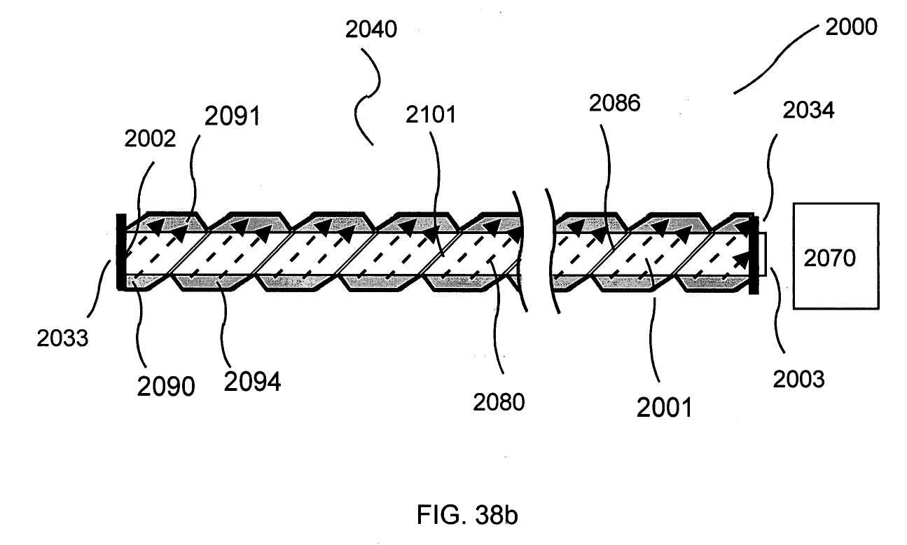

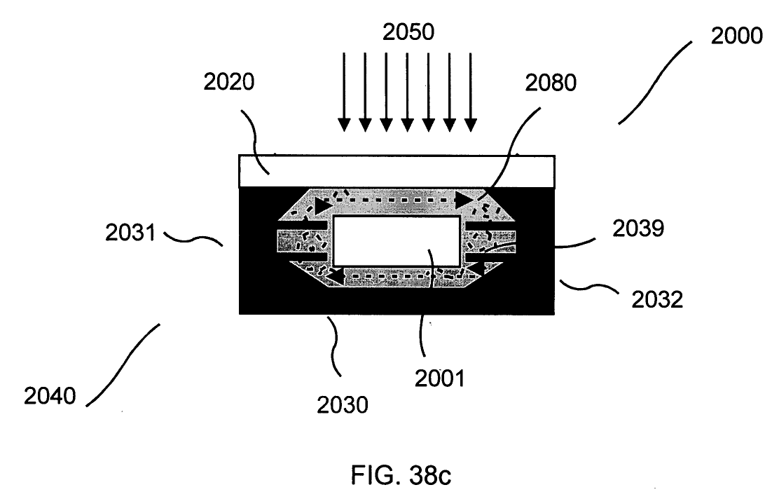

FIGS. 38a, 38b and 38c correspond to a bottom, top and end views of the mixing flow chamber according to one embodiment of the invention where the fluid is guided to flow in a spiral pattern 2080 around the waveguide 2001 and the fluid is mixed at the sides of the waveguide 2031, 2032 and 2039.

FIGS. 38a and b correspond to a bottom and top views showing the waveguide 2001, the direction of the flow (dashed arrows) 2080. The sample enters the chamber at the inlet 2060 and exits the chamber at the outlet 2061 at the bottom of the fluidic chip. Fluid flows in a spiral motion as follows: (a) fluid flows from the inlet 2060 to position 2090 over the top of the waveguide to position 2091 and then down to position 2092 at the bottom, and (b) fluid flows from position 2092 under the waveguide to position 2093 and then up to position 2904 at the top. This motion completes one cycle around the waveguide 2001 forming one segment of the flow chamber and the process repeats in additional segments until the end of the waveguide.

FIG. 38c represents an end cross-sectional view showing the waveguide 2001 and the fluid motion 2080 in dashed curves circling around the waveguide in a spiral. The fluid motion is guided by structures above and below the waveguide, not shown here, but is shown in FIGS. 38A and 38B. The fluid is passively mixed on the sides by three-dimensional structures 2039 on the sides of the flow-chamber 2031 and 2032. All the variations of inventions described earlier for FIGS. 20a-c, FIGS. 30a-c are also applicable, for example, to this multi-mixing flow chamber sensor embodiment.

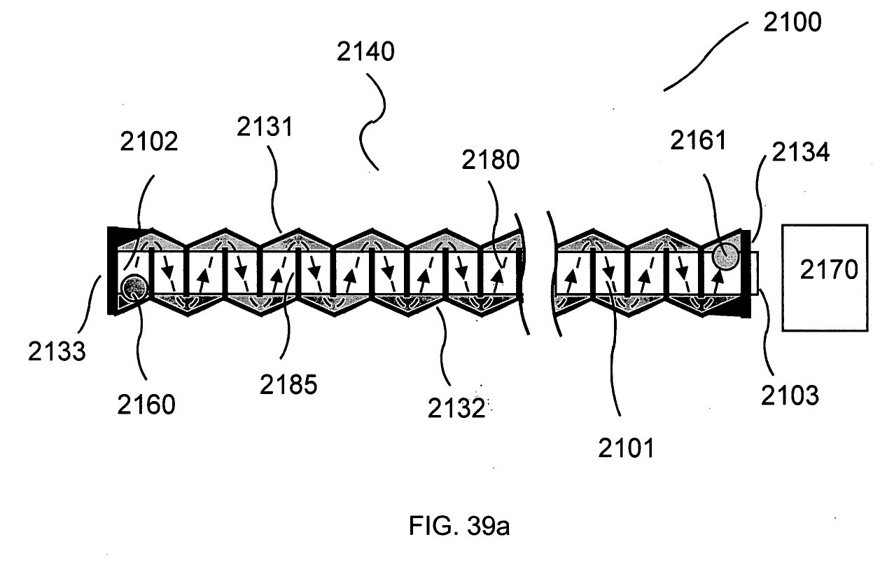

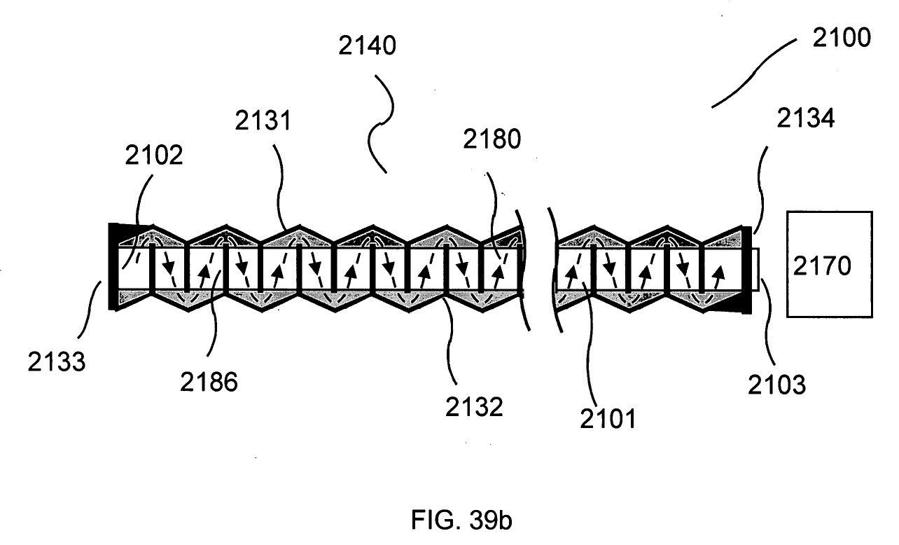

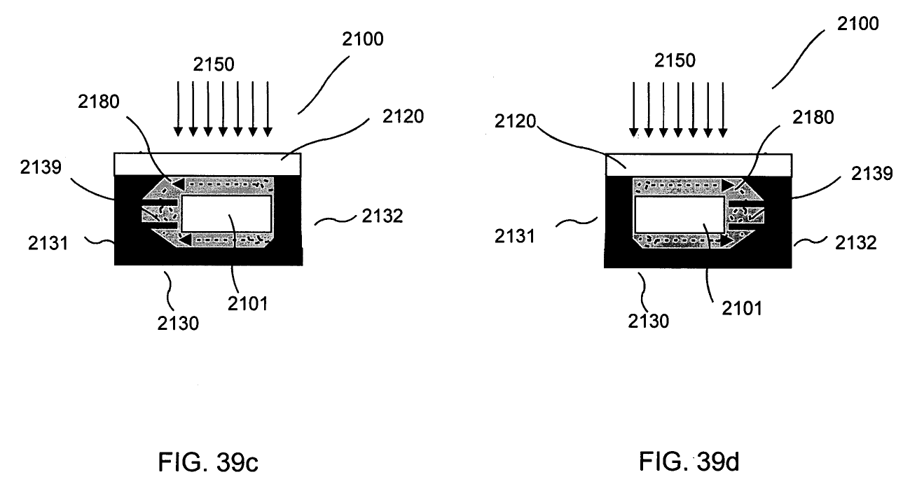

FIGS. 39a, 39b, 39c and 39dcorrespond to a bottom, top, and end views at one axial location and end view at another axial location of the mixing flow-through sensor according to one embodiment of the invention where the fluid is guided by structures 2185 and 2186 to flow in a zig-zag pattern 2180 across the top and bottom of the waveguide 2101, and the fluid is mixed at the sides of the waveguide 2131, 2132 and 2139.

FIGS. 39c and 39drepresent a cross-sectional end view at two different locations showing the waveguide 2101, shape of the flow chamber walls 2139, and the fluid motion in dashed curves 2180. All the variations of inventions described earlier for FIGS. 20a-cc, FIGS. 30a-c are also applicable, for example, to this multi-mixing flow chamber sensor embodiment.

Mixing flow waveguide sensor can also be achieved with an embodiment utilizing evanescent wave excitation. The excitation source propagates along the inside of the optical waveguide. The excitation light is not applied from the sides of the waveguide, but input into the waveguide at one end. All the previous description about the wall undulations are applicable to the evanescent wave excitation. In addition, the surfaces 220 in FIGS. 20b and 20c, the surfaces 620 in FIGS. 23a-d, the surfaces 720 in FIGS. 24a and 24b, the surfaces 820 in FIGS. 25a and 25b, the surface 920 in FIGS. 26, the surfaces 320 in FIGS. 27b and 27c, the surfaces 420 in FIGS. 28b and 28c, the surface 1120 in FIGS. 29, the surfaces 520 in FIGS. 30b and 30c, the surfaces 520a, 520b, 520c and 520d in FIG. 31, the surfaces 1220 in FIGS. 32a and 32c, the surfaces 1320a and 1320b in FIG. 33, surfaces 1420a, 1420b and 1420c in FIG. 34, and the surface 1720 in FIGS. 36 do not have to be clear and they too can have undulating shape to provide mixing.

It is understood that modifications which do not substantially affect the activity of the various embodiments of this invention are also included within the definition of the invention provided herein. Accordingly, the following examples are intended to illustrate but not limit the present invention.

### EXAMPLE I

### Cell Capture, Growth and Detection Using a Combined Immunological-Amplification Biosensor

This Example describes detection of water-borne E. coli using an integrated biosensor for the capture, growth and PCR amplification of bacteria analytes.

Enterohemorrhagic E. coli (e.g., E. coli O157:H7) has emerged as a serious problem in developed countries. This strain is one of the most common serotype of enterohemorrhagic E. coli (EHEC), and is responsible for numerous food-borne and water-borne infections worldwide. Symptoms include bloody diarrhea and kidney failure, which can be fatal. Enterohemorrhagic E. coli strains may be candidates for bioterrorism agents because of their virulence and the very small infectious dose. Epidemiological data suggests that consumption of relatively few cells (ca. 10) can result in infection. Traditional methods for detection of E. coli O157:H7, which rely on enrichment, plating on selective media, and identification via biochemical/serological testing, are time consuming and labor intensive. Recently, other immunological- and PCR-based methods have been developed. However, the limit of detection for both methodologies is approximately 100 cells/mL, which is inadequate. Consequently, these methods must be combined with concentration or enrichment prior to detection. In addition, neither immunological or PCR assays alone are definitive for enterohemorrhagic E. coli. Therefore, methodology currently in use for detection of E. coli O157:H7 includes culture-based isolation coupled with immunoassay for O157 and H7 antigens and DNA amplifications for multiple target genes. Accordingly, no single method is able to detect and quantify small numbers of E. coli O157:H7 from a large volume of water, and simultaneously confirm strain identity.

Described herein is a combined immunological-PCR biosensor system to provide an integrated solution. This single system is capable of isolating and concentrating E. coli O157 from water and determining their serotype, genotype and viability. Briefly, anti-O157 antibodies attached to the inner surface of capillary tubes allow for cell capture from a flowing stream of water (i.e., concentration). Subsequently, tubes are incubated with a second antibody conjugated with Cy-5 (sandwich assay), allowing for detection via the Integrating Waveguide Biosensor (Ligler et al., Anal. Chem. 74:713-719, 2002). Alternatively, capillary tubes can be filled with enrichment medium and incubated, resulting in growth of the captured viable cells within the tube. Tubes can then be analyzed via the Integrating Waveguide Biosensor, or cells lysed in the tube followed by real-time PCR analysis. Data will be presented demonstrating each of the assay components. Experiments are in progress to optimize each component and to integrate the components into a single system.

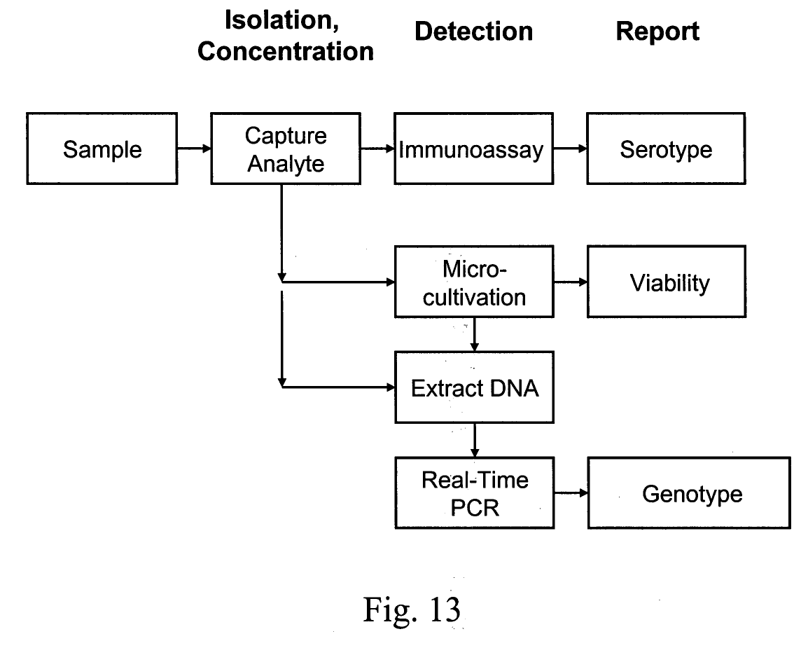

The biosensor system has integrated three components for the detection of E. coli O)157:H7: (1) Sample preparation: capture antibodies on the capillary surface allow for isolation and concentration of bacteria from water samples. (2) Detection: the capillary is subsequently subjected to immunoassay, microcultivation or cell lysis in the tube followed by real-time PCR analysis. (3) Data output: report bacterial serotype, genotype and viability. A schematic of this embodiment of the integrated biosensor system is shown in FIG. 13.

The instrument, integrating waveguide immunosensor, is based on illumination of an optical waveguide perpendicular to the length of the waveguide and a subsequent collection of the emitted fluorescence from the sandwich assay of the analyte at one end of the waveguide which can be a capillary tube as shown in FIG. 14 (see, for example, Ligler et al., supra, (2002). The emitted light is coupled very efficiently into the waveguide and the signal is integrated by the geometry of the sensing component. The emitted light can be collected on a single photo multiplier tube (PMT) or photodiode. Consequently, the signal from a relatively large surface is integrated and measured at a single-point.

E. coli strain O157 was captured by affinity binding on an integrated biosensor waveguide capillary. Briefly, glass waveguide capillary tubes (75 mm long, 1.661 mm O.D., 1.22 mm I.D.) were coated with anti-E. coli O157 monoclonal antibody (MAb) as described by Ligler et al., supra, (2002). Capillary tubes were incubated with 75 μl ofE. coli O157 (1.4×104 CFU/ml) at 25° C. for 1 h. Capillary tubes were subsequently washed with PBS to remove unbound cells and then treated with 0.1 M glycine buffer (pH 3.2) for 10 min to dissociate bound cells from the capillary surface. The dissociated cells were plated on MacConkey agar. Analysis of the capillary tubes showed that E. coli O157 cells were captured only in the capillary tubes coated with anti-O157.

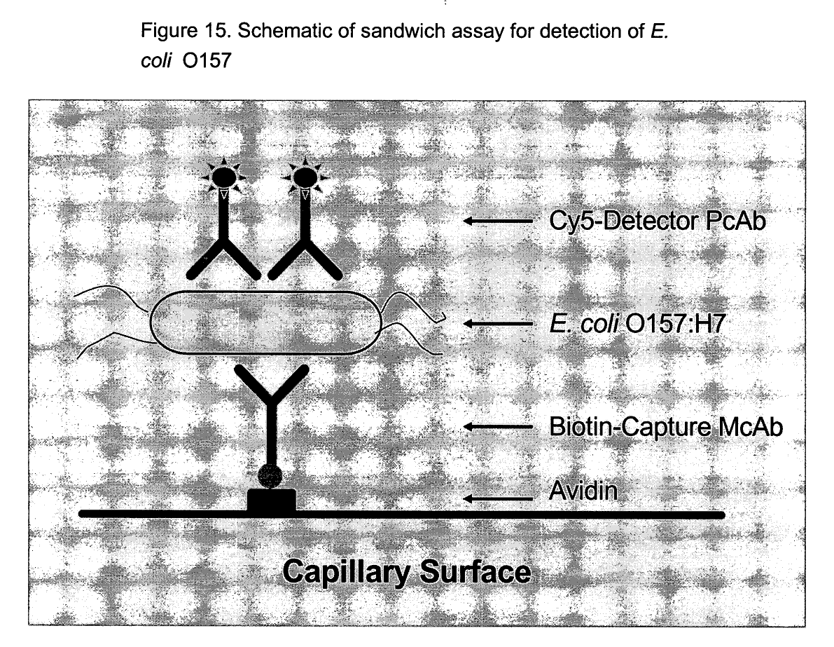

For detection of E. coli O157 an antibody sandwich assay was performed in the capillary tubes as shown in FIG. 15. The negative control tubes were exposed to all reagents except for the bacteria. The fluorescence signal (mean and SD, n=3) for the negative controls were 1.10 mV and 0.09 mV, respectively (FIG. 16). The threshold detection value (Mean+3SD) was set at 1.37 mV. As few as 83 E. coli O157:H7 cells (as determined by real time PCR, see below) generated a significant signal: 2.4 mV (S/N=2.18). The results of these detection measurements are presented in FIG. 16 and show that the biosensor sensitivity for E. coli O157 was <10² cells/capillary.

Genetic material was used as a basis for identifying the presence or amount of captured target cell analytes. Briefly, three lysis buffers were tested to extract DNA template from the capillary tubes. After binding of E. coli O157, capillary tubes were incubated with the lysis buffers at 37° C. or 95° C. for 10 min. The cell lysate was directly used for amplification of lacZ by real time PCR to estimate cell numbers. The best recoveries were obtained with Buffer A as shown below in Table 1. Lysis buffer A consists of Triton X-100 detergent (Sigma). Lysis buffer B consists of a NP-40 detergent (Sigma), whereas lysis buffer C corresponds t AL lysis buffer (Qiagen kit).

TABLE 1
Recovery of E. coli O157 using various lysis buffer.

Recovered E. coli O157^{a}

| Lysis Buffer | 37° C. | 95° C. |
|---|---|---|
| A | 3640 | 3318 |
| B | 2290 | 3094 |
| C | 350 | ND |

^{a}Estimated bacterial cell numbers.
ND, not determined

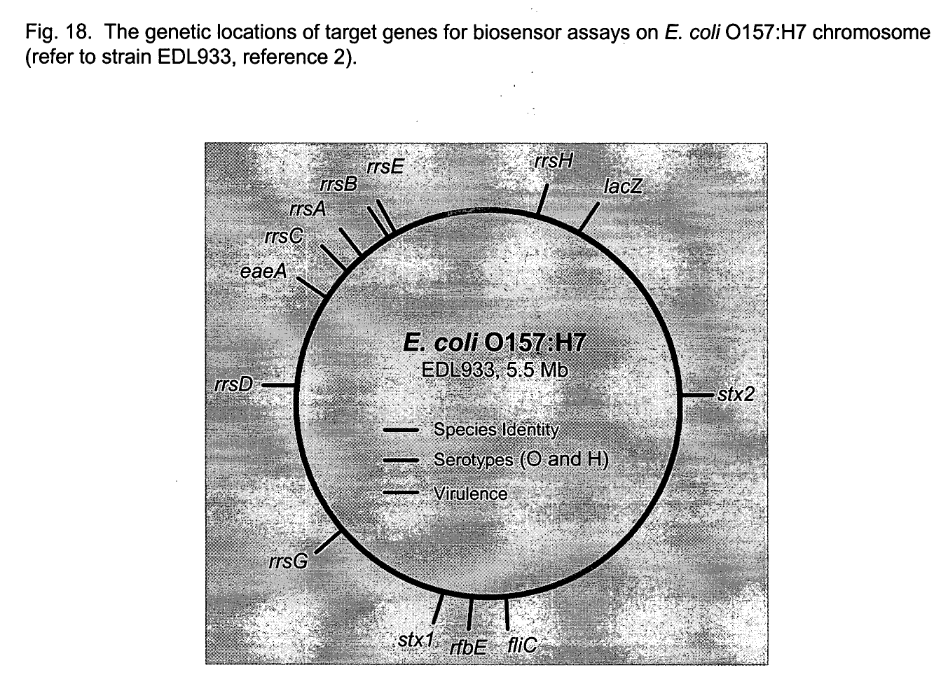

The lacZ gene was used for real-time PCR to estimate E. coli O157 cell numbers on the capillary surface. The plot in FIG. 17 shows a real-time amplification of a series of the lacZ standard (101-106/μl) and E. coli O157 DNAs extracted from the capillary sets (A-D). Multiple genes on the E. coli O157:H7 genome can be used for confirmation of bacterial species (16S rRNA and lacZ), serotype (O157:H7) (rjbE and fliC) and virulence (stx1, stx2 and eaeA) (FIG. 18).

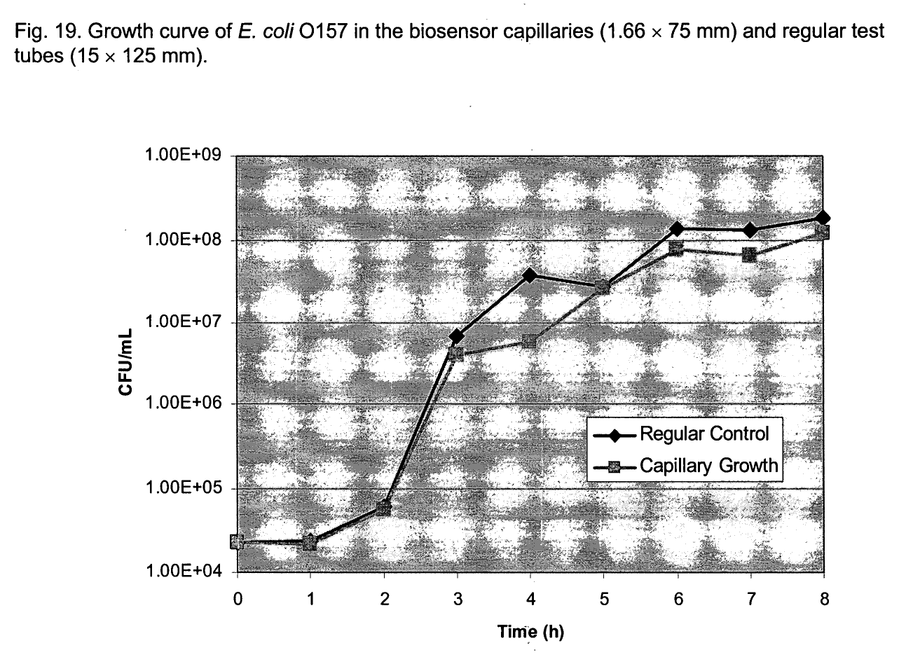

Microcultivation of captured E. coli O157 cells also was performed in the capillary tubes. Briefly, the growth characteristics of E. coli O157 in the capillary tubes were determined as a method for assessing viability of captured cells. E. coli O157 was grown at 44° C. in MLB-Y in capillary tubes and in regular test tubes. The growth curve in capillary tubes was similar to that in regular test tubes. After 6 h, the bacteria entered stationary phase. The results are shown in FIG. 19 where the curve indicates that 3-6 h of enrichment (microcultivation) is sufficient to assess viability.

Current methods for the detection of E. coli O157:H7 require different assays. The combined immunological-PCR biosensor described here has integrated multiple assays into a single system which allows for determination of bacterial serotype, genotype and viability simultaneously. The data presented here demonstrate the concept that the biosensor system is capable of directly capturing and concentrating the bacteria from water with subsequent detection in a fluorescence sandwich assay and real time PCR.

The above results demonstrate the use of a combined immunological-PCR biosensor system for rapid and sensitive detection, and confirmation, of E. coli O157:H7 in water samples. This system can be adapted for the detection of other infectious agents as well as for other biological particles or cells.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

What is claimed is:

1. A bioprocessor, comprising an integrated capture chamber having an analyte recognition coating and a structure supporting analyte detection and target nucleic acid detection.

2. The bioprocessor of claim 1, wherein said capture chamber further comprises a waveguide.

3. The bioprocessor of claim 1, wherein said capture chamber comprises a capillary tube.

4. The bioprocessor of claim 1, wherein said capture chamber comprises a mixing flow chamber.

5. The bioprocessor of claim 1, wherein said analyte recognition coating comprises an antibody.

6. The bioprocessor of claim 1, wherein said analyte detection comprises a secondary binding reagent.

7. The bioprocessor of claim 1, wherein said target nucleic acid detection comprises nucleic acid probe hybridization or nucleic acid amplification.

8. The bioprocessor of claim 1, wherein said target nucleic acid detection is performed in a second chamber.

9. The integrated bioprocessor of claim 1, further comprising an illumination source.

10. The integrated bioprocessor of claim 1, further comprising a radiation detector.

11. The integrated bioprocessor of claim 1, further comprising a microfluidics handling system.

12. The bioprocessor of claim 1, further comprising structure supporting analyte growth.

13. A biosensor, comprising an integrated capture chamber having an analyte recognition coating, an illumination source, a radiation detector and a structure supporting analyte detection and target nucleic acid detection.

14. The biosensor of claim 13, wherein said capture chamber further comprises a waveguide.

15. The biosensor of claim 13, wherein said capture chamber comprises a capillary tube.

16. The biosensor of claim 13, wherein said capture chamber comprises a mixing flow chamber.

17. The biosensor of claim 13, wherein said analyte recognition coating comprises an antibody.

18. The biosensor of claim 13, wherein said analyte detection comprises a secondary binding reagent.

19. The biosensor of claim 13, wherein said target nucleic acid detection comprises nucleic acid probe hybridization or nucleic acid amplification.

20. The biosensor of claim 13, wherein said target nucleic acid detection is performed in a second chamber.

21. The integrated biosensor of claim 13, further comprising a microfluidics handling system.

22. The bioprocessor of claim 13, further comprising structure supporting analyte growth.
